(19) United States
(12) Patent Application Publication
(10) Pub. No.: US 2013/0074221 A1
(43) Pub. Date: Mar. 21, 2013

(54) AUTOLUMINESCENT PLANTS INCLUDING THE BACTERIAL LUX OPERON AND METHODS OF MAKING SAME

(71) Applicant: Alexander Krichevsky, St. Louis, MO (US)

(72) Inventor: Alexander Krichevsky, St. Louis, MO (US)

(73) Assignee: BIOGLOW, L.L.C., St. Louis, MO (US)

(21) Appl. No.: 13/580,922

(22) PCT Filed: Feb. 25, 2010
(86) PCT No.: PCT/US2010/025366

§ 371 Date: Nov. 6, 2012

(51) Int. Cl. C12N 15/82

(52) U.S. Cl. CPC C12N 15/8242
USPC 800/298; 435/320.1

(57) ABSTRACT

In one aspect, the invention relates to a transgenic autoluminescent plant including an expressible heterologous nucleotide sequence comprising a bacterial LUX operon, which includes LUX A. LUX B. LUX C. LUX D. LUX b. And LUX G genes, wherein the heterologous nucleotide sequence is expressed to render the plant autonomously luminescent.

### INCORPORATION BY REFERENCE

International Patent Application No. PCT/US2008/009310 entitled, “Bioluminescent Plants Comprising Bacterial Lux Operon and Methods of Making Same.” filed Jul. 31, 2008, and U.S. Provisional Application No. 60/953,337, filed Aug. 1, 2007, are hereby incorporated by reference in their entirety.

### BACKGROUND OF THE INVENTION

Non-bacterial organisms such as plants that are capable of autoluminescence would be useful for many purposes, such as for environmental, research, and aesthetic applications. However, such organisms have not been readily achieved for many reasons. For example, the genes and mechanisms responsible for autoluminescence are complex. Attempts to incorporate complex metabolic pathways, such as those involved in light emission, into transgenic plant organisms have been hampered by limitations of genetic engineering.

Previous attempts of plant genetic engineering to achieve luminescence have resulted in significant disadvantages. For example, expressing luciferases in plant tissues typically require contact of the tissue with a substrate (e.g., luciferin) to emit light. The light emission is typically temporally limited, lasting only a few hours or minutes. Some luciferin substrates are toxic, highly unstable, and/or expensive.

Accordingly, plants that are capable of being autonomously bioluminescent (i.e., autoluminescent) and methodologies that enables incorporation of complex metabolic pathways into plants are needed.

### SUMMARY OF THE INVENTION

The present invention addresses these and other objectives.

In one aspect, the invention relates to a transgenic bioluminescentautoluminescent plant cell. The plant cell includes a heterologous nucleotide sequence comprising a bacterial LUX operon, which includes LUX A, LUX B, LUX C, LUX D, LUX E, and LUX G genes, wherein the heterologous nucleotide sequence is operably linked to a truncated Prrn promoter, and wherein the heterologous nucleotide sequence is integrated in a plastid genome.

In another aspect, the invention relates to a kit that includes a seed for generating a transgenic autoluminescent plant cell. The plant cell includes a heterologous nucleotide sequence, which includes a bacterial LUX operon. The bacterial LUX operon includes LUX A, LUX B, LUX C, LUX D, LUX E, and LUX G genes, wherein the heterologous nucleotide sequence is operably linked to a truncated Prrn promoter, and wherein the heterologous nucleotide sequence is integrated in a plastid genome. The kit further includes a plant transformation vector.

In a further aspect, the invention relates to a vector system. The vector system includes a plastid transformation vector having a first heterologous nucleotide sequence comprising a bacterial LUX operon, which includes LUX A, LUX B, LUX C, LUX D, LUX E, and LUX G genes, wherein the heterologous nucleotide sequence is operably linked to a first promoter, and wherein the heterologous nucleotide sequence is capable of being incorporated into a plastid genome. The vector system also includes a vector having a second heterologous nucleotide sequence operably linked to a second promoter.

In yet a further aspect, the invention relates to a vector system. The vector system includes a plastid transformation vector having a first heterologous nucleotide sequence, which includes any live of the following LUX A, LUX B, LUX C, LUX D, LUX E, and LUX G genes, wherein the heterologous nucleotide sequence is operably linked to a truncated Prrn promoter, and wherein the heterologous nucleotide sequence is capable of being incorporated into a plastid genome. The vector system also includes a vector having a second heterologous nucleotide sequence, which includes a plastid targeting sequence and the sixth LUX gene operably linked to a second promoter.

### BRIEF DESCRIPTION OF DRAWINGS

FIG. 1: A) Cultures of Photobacterium NZ-11 growing in petri dishes from Corbis; B) Cultures of Photobacterium Phosphoreum. [from The Danish Institute for Fisheries Research]; C) Conserved genetic structure of the LUX operon in different luminous bacteria species: abbreviations: Pp: Photohacterium phosphoreum, Pl: Pholobacterium leiognatlhi, subtypes 1 and 2, Vf: Vibrio fischeri, Vh: Vibrio harveyi, Xl: Xenorhabdus luminescens [E. Meighen, Microbiol Rev, 1991]; D) Biochemistry of bacterial luminescence reaction, (from: “The Biochemistry and Molecular Biology of Bacterial Bioluminescence” by Y-C. Lin and E. Meighen)

FIG. 2: The chloroplast genome and schematic structure and prokaryotic functional features of plastid transformation vectors. The homologous recombination machinery of the chloroplast promotes targeting of the integrating DNA into a specific genome area (e.g. the TrnI/TrnA locus) via homology with sequences flanking the transgene expression cassette. Polycistronic gene expression machinery allows expression of several transgenes from a single operon-like structure, simplifying construction of the multigene transformation vector and permitting integration of multiple transgenes in a single transformation step. Recombinant protein expression levels, which are typically significantly higher for chloroplast than for nuclear transgenes, are further increased as a result of copy correction, which causes duplication of the expression cassette to the homologous site on the opposite inverted repeat (i.e. from IR_{A} to IR_{B}).

FIG. 3: Schematic illustration of the Genetic Relay Assay, where T7 RNA polymerase protein expression is driven by a tissue-specific or circadian rhythm or otherwise inducible (stress, heavy metal, etc) promoter in the nucleus. When the aforementioned promoter is activated, the T7 RNA polymerase protein will be transcribed and targeted to a plastid (e.g., a chloroplast) using N-terminally fused plastid transit peptide. The LUX genes in the chloroplast will be driven by the T7 promoter, to which T7 RNA polymerase binds and thus activates LUX transcription. Hence, activation of the LUX operon is indirect.

FIG. 4: Schematic illustration of the Genetic Complementation Assay, where one of the genes required for the luminescence (such as luciferase subunit LuxA) is expressed from an inducible promoter in the nucleus and targeted into the plastid using transit peptide. While rest of the genetic machinery required for the luminescence is constitutively expressed in the plastid, for instance driven by the truncated Prrn promoter, light emission will occur only when the light emission machinery is complemented by the LUX subunit targeted from the nucleus, which in turn is regulated by an inducible promoter.

FIG. 5: Genetic maps of pSAT4-MCS (A) and pCAS3 vectors (B).

FIG. 6: Genetic map (A) and actual experimental restriction digest (B) of the fully constructed pCAS3-aadA vector, resolved on 1% agarose gel, yielding the Prrn promoter (AgeI/NcoI digest, approx. 100 bp fragment), aadA gene (NcoI/BglII digest, approx. 800 bp fragment) and 35S terminator (BamHI/NotI digest, approx. 230 bp fragment). C) Genetic map and (D) actual experimental restriction digest of the fully constructed pCAS3-aadA-LUX operon vector, demonstrating LUX operon cloned into the pCAS3-aadA backbone (EcoRI digest, yielding LUX operon fragment of approx. 6.5 kb). The marker is 1 kb Plus DNA ladder (Invitrogen).

FIG. 7: Genetic maps of (A) pCAS3-LUX-rps12/TrnV and (B) pCAS3-LUX-TrnI/TrnA vectors and (C) the actual experimental restriction digest of the fully constructed aforementioned vectors, resolved on 1% agarose gel, demonstrating rps12/TrnV homologues recombination sequences (AgeI and NotI digests respectively, yielding approx. 2.0 kb fragments) cloned into pCAS3-LUX-rps12/TrnV vector (left side of the C panel), and TrnI/TrnA homologues recombination sequences (AgeI and NotI digest respectively, yielding approx. 1.6 kb fragments) cloned into pCAS3-LUX-TrnI/TrnA vector (right side of the C panel). The presence of approx. 6.5 kb LUX operon is shown by EcoRI digests. The marker is 1 kb Plus DNA ladder (Invitrogen).

FIG. 8: A) Early prototyping of pCAS3-aadA and pCAS3-aadA-LUXoperon vectors in E. coli. DH5α cells, normally sensitive to spectinomycin, have been transformed with pCAS3-aadA (left panel side) and pCAS3-aadA-LUXoperon (right panel side) vectors and grown on LB agar supplemented with 100 μg/ml of spectinomycin. Both vectors conferred spectinomycin resistance to the DH5α cells (upper panel), and pCAS3-aadA-LUXoperon cells also emitted visible light in the dark (lower panel). B) Transplastomic tobacco plant.

FIG. 9: A) Schematic representation of the PCR-amplified regions used in identification of the transplastomic plants. Expected PCR fragment sizes and primer numbers are demonstrated: for instance, primers #78 and #104 used to amplify rps12 junction region resulting from the vector integration within the chloroplast rps12 gene; expected PCR fragment size is 2.35 kb. B) Actual experimental PCR fragments, resolved on 1% agarose gel, obtained during identification of transplastomic plants generated using pCAS3-LUX-rps12/TrnV chloroplast transformation vector. Left lane in each pair of lanes on the agarose gels is wild type plant DNA, used as negative control; the right lane is the transplastomic plant DNA: primers pair used for each wild type/transplastomic pair shown above and correspond to the scheme in (A). Primers #73 and #79 are designed to amplify a region of native chloroplast genome and used as positive controls of the PCR reaction of both wild type and transgenic plants. The marker is 1 kb Plus DNA ladder (Invitrogen).

FIG. 10: Light emission by the transplastomic plant tissue as detected by the scintillation counter (LS 6500 Multi-purpose scintillation counter, Beckman Coulter) for transplastomic plants generated using (A) pCAS3-LUX-rps12/TrnV and (B) pCAS3-LUX-TrnI/TrnA vectors; wild-type tobacco tissue used to measure baseline noise. C) Transplastomic plants generated using pCAS3-LUX-TrnI/TrnA (upper panel) exposed to a photographic film (lower panel). Please note a defined and focused light emission around the transplastomic tissue, while no light emission has been detected with the wild-type tissue. The exposure foci coincide precisely with the position of the transplastomic tissue on the plate. With this, for the larger transplastomic tissue section (right lower side of the transplastomic tissue plate), light emission was not homogeneous across the whole specimen and has been concentrated in an 8-shaped two distinct foci (marked with an arrows), likely resulting from heteroplastomy of the initial transplastomic shoots.

FIG. 11. A) Photograph of LUX-TrnI/TrnA plants taken in dark room using hand-held consumer camera [Nikon D200; AF-S Micro Nikkor 105.0 mm 1:2.8 G ED lens; exposures 5 min at f/4.5, 105 mm focal length, ISO 3200]; B) A photograph similar to (A) demonstrating side-by-side comparison of LUX transplastomic with wild type tobacco plants in regards to light emission [upper panel exposure taken with lights on; lower panel exposure taken with lights off]; C) Addition of decanal to final concentration of 2 mM doubles light emission from the transplatomic LUX tissue.

FIG. 12. Promoters alignment to demonstrate sequence differences, performed using ClustalW2.

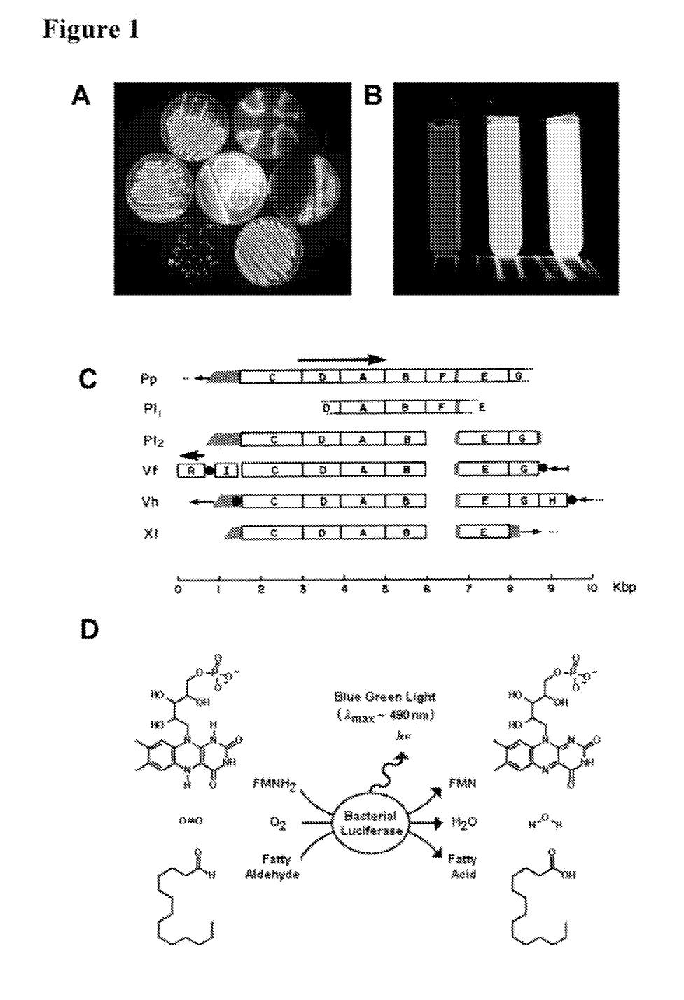

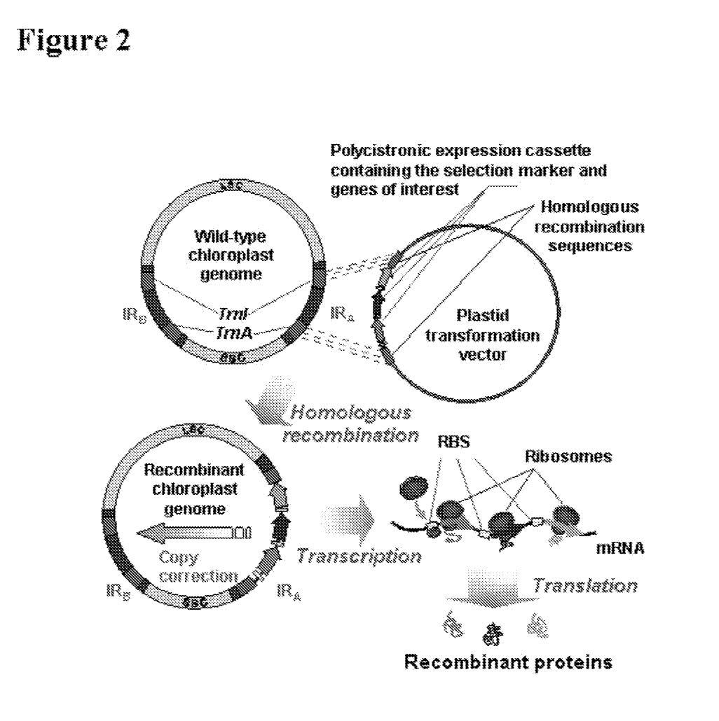

### DETAILED DESCRIPTION OF THE INVENTION

### Transgenic Autoluminescent Plant

In one aspect, the invention relates to a transgenic autoluminescent plant cell. The plant includes a heterologous nucleotide sequence, which includes a bacterial LUX operon. The LUX operon includes LUX A, LUX B, LUX C, LUX D, LUX E, and LUX G genes (“the six LUX genes”). The heterologous nucleotide sequence is operably linked to a truncated Prrn promoter, and the heterologous nucleotide sequence is integrated in a plastid genome.

The terms “transgenic,” “transformed,” “transfected” as used herein includes any cell, cell line, callus, tissue, plant tissue, or plant into which a nucleic acid heterologous to the host cell has been introduced. The term “transgenic” as used herein does not encompass an alteration of the genome (chromosomal or extra-chromosomal) by conventional plant breeding methods or by naturally occurring events, such as random cross-fertilization, non-recombinant viral infection, non-recombinant bacterial transformation, non-recombinant transposition, or spontaneous mutation. The term “transgenic plant” refers to a plant or plant tissue that contains an inheritable heterologous nucleotide sequence.

The term “autonomously luminescent” or “autoluminescent” as used herein refers to luminescence that occurs in a plant or plant tissue, in which energy from a chemical reaction is transformed into light energy. The transgenic plant or plant tissue autonomously emits light, without the need of external manipulation, such as, for example, without the need to apply external substrates to said transgenic plant or plant tissue. The term “autoluminescent” further refers to the production of light in a recombinant plant or plant tissue engineered to contain chemical compounds necessary for luminescence in the plant or plant tissue. Preferably, the transgenic plant is “stably” autoluminescent, which refers to the introduction and integration of a heterologous nucleotide sequence for autoluminescence into the genome of a transfected cell.

The term “plant” is used broadly herein to refer to a eukaryotic organism containing a plastid, and being at any stage of development. The term “plant” as used herein refers to a whole plant or a part of a plant (e.g., a plant cutting, a plant cell, a plant cell culture, a plant organ, a plant seed, and a plantlet), a seed, a cell- or a tissue-culture derived from a plant, plant organ (e.g. embryos, pollen, ovules, seeds, leaves, flowers, branches, fruit, kernels, ears, cobs, husks, stalks, roots, root tips, anthers, etc.).

Any plant may be used for the invention. For example, Nicotiniana benthamiana, Arabidopsis thaliana, or Nicotiana tabacum (tobacco) can be used, as they are frequently employed as model organisms in plant research and a large amount of data regarding their biology has been accumulated. Also, a good model organism for the autoluminescent plants of the present invention are plants which intrinsically express large amounts of FMNH₂, such as, for example, asparagus or chard. To minimize costs, the luminescent system from several highly luminescent bacterial species, such as Photobacterium leiognathi or Vibrio harveyi, as well as Shewanella hanedui, is transferred into several plant species. Since this approach requires application of essentially same technology in parallel on several gene transfers, this strategy does not significantly increase costs of generation of autoluminescent plants.

Other preferred plants include ornamental plants, useful or ornamental trees, flowers, cut flowers, shrubs, or turf. Exemplary plants include carnations, chrysanthemums (pompon), lilies, gerbera, snapdragons, roses, tulips, petunias, daises, geranium, argyranthemum, begonia, coleus, tulips, gladioli, delphinium, lisianthus, iris, orchids, alstroemaria, etc.

The transgenic autoluminescent plant, as used herein, includes at least one plant cell. A “plant cell” refers to any cell of a plant, either taken directly from a seed or plant, or derived through culture from a cell taken from a plant. A plant cell includes, for example, cells from undifferentiated tissue (e.g., callus), plant seeds, propagules, gametophytes, sporophytes, pollen, microspores, and embryos.

A plant cell typically contains a “plastid,” which refers to an organelle with its own genetic machinery in a plant cell. Examples of a plastid include chloroplasts, chromoplasts, etioplasts, gerontoplasts, leucoplasts, proplastids, amyloplasts, elaioplasts, etc. The plastids of higher plants are an attractive target for genetic engineering. Plant plastids are major biosynthetic centers that, in addition to photosynthesis, may be responsible for production of important compounds such as amino acids, complex carbohydrates, fatty acids, and pigments. Plastids are derived from a common precursor known as a proplastid and thus the plastids present in a given plant species all have the same genetic content. Plant cells may contain anywhere between 500-10,000 copies of a 120-160 kilobase circular plastidal genomes and therefore plant cells may be engineered to contain multiple copies of a particular gene of interest, integrated within the aforementioned plastidal genome, which potentially can result in very high levels of transgene expression. In addition, plastids of most plants are maternally inherited. Consequently, unlike transgenes expressed in the cell nucleus, heterologous genes expressed in plastids are not pollen disseminated and therefore, a trait introduced into a plant plastid will not be transmitted by pollen to wild-type relatives, thereby preventing transgene escape.

The transgenic autoluminescent plant further includes an expressible heterologous nucleotide sequence. The term “expressible,” “expressed,” and variations thereof refer to the ability of a cell to transcribe a nucleotide sequence to mRNA and translate the mRNA to synthesize a peptide that provides a biological or biochemical function. Preferably, the cell is a plant cell.

As used herein, “heterologous” refers to that which is foreign or non-native to a particular host or genome. Accordingly, a “heterologous nucleotide sequence” or “transgene” refers to a nucleotide sequence that originates from a species foreign to the host organism, or if the nucleotide sequence originates from the same species as the host, the nucleotide sequence is substantially modified from its native form in composition and/or genomic locus by deliberate genetic manipulation. The term “nucleotide sequence” refers to a sequence of two or more nucleotides, such as RNA or DNA. A “heterologous protein” refers to a protein that is foreign or non-native to a host cell and is typically encoded by a heterologous nucleotide sequence.

### The LUX Operon

The LUX operon contains 6 luminescence genes in the following order: C-D-A-B-E-G. The Lux A and B genes encode luciferase subunits. The Lux C, D and E genes encode fatty-reductase complex which produces aldehyde for the reaction. The Lux G gene encodes an exchange factor, facilitating FMNH₂ turnover.

The enzymatic complex, encoded by the Lux CDE genes, diverts a range of fatty acids from the basic fatty acids biosynthesis cycle, converting them to the aldehyde substrate and channeling them to the luminescence reaction. The other substrate, the FMNH₂, is naturally produced in bacteria, as well as plant plastids. One of the pathways for FMNH₂ production in the luminescent bacteria is encoded by the RIB operon (SEQ ID NO: 1), in some species immediately adjacent to the LUX operon.

In one embodiment, the heterologous nucleotide sequence includes a bacterial LUX operon. Use of the complete bacterial LUX operon allows for intrinsic luminescence (or “autoluminescence”), which refers to the ability of a transgenic cell to contain all of the required elements for production of light, without the requirement for exogenous addition of chemical compounds or substrates, and/or any other kind of external manipulation.

The term “operon” refers to a nucleotide sequence which codes for a group of genes transcribed together. The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression. Some genes can be transcribed into mRNA and translated into polypeptides (structural genes); other genes can be transcribed into RNA (e.g., rRNA, tRNA); and other types of genes function as regulators of expression (regulator genes).

The term “LUX operon” as used herein refers to an operon that includes at least six genes for autoluminescence. The six genes include LUX A, LUX B, LUX C, LUX D, LUX E, and LUX G genes.

For purposes of the invention, the genes corresponding to the LUX operon, and any other gene required for proper functioning of bacterial luciferase in a plant, are isolated from the genome of luminescent bacteria. For example, the LUX operon and LUX A, LUX B, LUX C, LUX D, LUX E, and LUX G genes may be derived from any luminescent bacteria that express the LUX genes to generate luminescence.

Examples of a nucleotide sequence encoding the full LUX operon is presented in GenBank under accession numbers AY341062 (Vibrio fischeri [Vibrio fischeri strain ATCC 7744 lux operon, complete sequence](SEQ ID NO: 2); EU192082 (Vibrio harveyi [Vibrio harveyi BCB440 lux operon, complete sequence]) (SEQ ID NO: 3); AF403784 (Photorhabdus luminescens, (formally referred as Xenorhabdus luminescens [Photorhabdus luminescens lux operon, complete sequence]) (SEQ ID NO: 4); and AB261992 (Shewanella hanedai [Shewanella hanedai lux operon (luxC, luxD, luxA, luxB, luxE, luxG) genes and flanking regions, strain: NCIMB 2157]) (SEQ ID NO: 5); and M63594 (Photobacterium leiognathi [Photobacterium leiognathi lux operon (luxC, luxD, luxA, luxB, luxE, luxG) genes, complete cds]) (SEQ ID NC): 6); and DQ988873 (Photobacterium phosphoreum [Photobacterium phosphoreum strain ATCC 11040, complete LUX and RIB operons]) (SEQ ID NO: 7).

Examples of a nucleotide sequence encoding LUX A, LUX B, LUX C, LUX D, LUX E, and LUX G genes are included in the nucleotide sequences encoding the full LUX operon, listed above. For example, the following LUX genes were derived from GenBank accession number M63594 (Photobacterium leiognathi [Photobacterium leiognathi lux operon (luxC, luxD, luxA, luxB, luxE, luxG) genes, complete cds]) (SEQ ID NO: 6): LUX A (SEQ ID NO: 8), LUX B (SEQ ID NO: 9). LUX C (SEQ ID NO: 10). LUX D (SEQ ID NO: 1), LUX E (SEQ ID NO: 12), and LUX G (SEQ ID NO: 13) genes.

Further examples of a LUX E gene is presented in GenBank accession number M62812 for Vibrio fischeri [Vibrio fischeri LuxE gene, partial cds; and LuxG gene, complete cds]) (SEQ ID NO: 14). Further examples of a LUX G gene is presented in the sequences of SEQ ID NO: 15 (Photobacterium leiognathi (derived from GenBank #M63594); SEQ ID NO: 16 (Photobacterium phosphoreum (derived from DQ988873); SEQ ID NO: 17 (Vibrio harveyi (derived from EU192082); SEQ ID NO: 18 (Vibrio fischeri (derived from M62812); and SEQ ID NO: 19 (Shewanella hanedai (derived from AB261992).

The nucleotide sequence of the LUX operon and LUX A, LUX B, LUX C, LUX D, LUX E, and LUX G genes may be derived from wild-type organisms. Wild-type refers to the normal gene or organism found in nature without any known mutation. Other nucleotide sequences within the invention include a nucleotide sequence that encodes variants of LUX A, LUX B, LUX C, LUX D, LUX E, and LUX G proteins, and a nucleotide sequence that encodes mutant forms, recombinant forms, or non-naturally occurring variant forms of these proteins.

In some exemplary embodiments, the heterologous nucleotide sequence includes additional genes related to metabolism of luciferase substrates, such as, for example, Vibrio harveyi FRP gene.

### Plastid Targeting Sequences

In another embodiment, the heterologous nucleotide sequence includes a plastid targeting sequence. A “plastid targeting sequence” as used herein refers to a nucleotide sequence that encodes a polypeptide sequence, which can direct a second polypeptide to a plastid of the plant cell. Preferably, the plastid targeting sequence is a chloroplast targeting sequence.

It is known in the art that non-chloroplast proteins may be targeted to the chloroplast by use of protein fusions with a peptide encoded by a chloroplast targeting sequence. For example, luciferase genes of a heterologous nucleotide sequence may be fused with a plastid targeting sequence. When the luciferase gene is expressed, the targeting sequence is included in the translated polypeptide. The targeting sequence then directs the polypeptide into a plastid, such as a chloroplast.

Typically, the chloroplast targeting sequence encodes a polypeptide extension (called a chloroplast transit peptide (CTP) or transit peptide (TP)). The polypeptide extension is typically linked to the N-terminus of the heterologous peptide encoded by the heterologous nucleotide sequence.

Examples of a chloroplast targeting sequence include a sequence that encodes the tobacco ribulose bisphosphate carboxylase (Rubisco) small subunit (RbcS) transit peptide, Arabidopsis thaliana EPSPS chloroplast transit peptide, the Petunia hyhrida EPSPS chloroplast transit peptide, and the rice rbcS gene chloroplast targeting sequence.

Further examples of a chloroplast target peptide include the small subunit (SSU) of ribulose-1,5-biphosphate carboxylase, and the light harvesting complex protein I and protein II. Incorporation of a suitable chloroplast targeting peptide has been shown to target heterologous protein sequences to chloroplasts in transgenic plants. Those skilled in the art will recognize that various chimeric constructs can be made, if needed, that utilize the functionality of a particular CTP to import a given gene product into a chloroplast.

Other CTPs that may be useful in practicing the present invention include PsRbcS-derived CTPs (Pisum sativum Rubisco small subunit CTP); AtRbcS CTP (Arabidopsis thaliana Rubisco small subunit 1A CTP; CTP1); AtShkG CTP (CTP2); AtShkGZm CTP (CTP2synthetic; codon optimized for monocot expression); PhShkG CTP (Petunia hybrida EPSPS; CTP4; codon optimized for monocot expression); TaWaxy CTP (Triticum aestivum granule-bound starch synthase CTPsynthetic, codon optimized for corn expression): OsWaxy CTP (Oryza sativa starch synthase CTP); NtRbcS CTP (Nicotiana tabacum ribulose 1,5-bisphosphate carboxylase small subunit chloroplast transit peptide); ZmAS CTP (Zea mays anthranilate synthase alpha 2 subunit gene CTP); and RgAS CTP (Ruta graveolens anthranilate synthase CTP). Other transit peptides that may be useful include maize cab-m7 signal sequence and the pea (Pisum sativum) glutathione reductase signal sequence.

Additional examples of such targeting sequences may include: spinach lumazine synthase (SEQ ID NO: 20), Chlamydomonas ferredoxin (SEQ ID NO: 21), and Rubisco activase (SEQ ID NO: 22) transit peptides, and others.

The chloroplast targeting sequence may be used to target any peptide encoded by a heterologous nucleotide sequence to the chloroplast or other plastid. In one embodiment, the chloroplast targeting sequence is linked to a 5′- or a 3′-end of the LUX A, LUX B, LUX C, LUX D, LUX E, or LUX G genes. In another embodiment, the chloroplast targeting sequence is linked to a 5′- or a 3′-end of a gene encoding a fluorescent protein.

### Vectors

In one embodiment, the heterologous nucleotide sequence can be placed in a single vector. For example, the heterologous nucleotide sequence can include the six LUX genes in a single vector. In another embodiment, a heterologous nucleotide sequence encoding one of the six LUX genes can be placed in a different vector for each LUX gene, resulting in multiple different vectors. The heterologous nucleotide sequence can additionally include at least one gene encoding a cofactor for enhancing autoluminescence.

The term “vector” as used herein refers to a vehicle used for introduction of a nucleotide sequence into a host. A vector may be a plasmid, cosmid, phage, transposon, virus, or any other suitable vehicle. Preferably, the vector is a plasmid. A vector may include regulatory sequences useful for expression of a gene product in a host, including but not limited to a promoter, ribosomal binding site, and termination sequences. In one preferred embodiment, the vector is a vector for transforming a plastid as described below in another aspect of the invention.

Numerous vectors are suitable for stable transformation of a plant cell or a plastid. Accordingly, the LUX genes may be delivered into nuclear or chloroplast genomes.

In one embodiment, for the transformation of nuclear host DNA, the vector is a binary vector. A “binary vector” refers to a vector that includes a modified T-region from Ti plasmid, which allows replication in E. coli and in Agrobacterium cells, and usually includes selection marker genes. Preferably, the vector is a binary pPZP-RCS vector, assembled employing expression cassettes derived from the pSAT vectors (Tzfira T, Tian G W, Lacroix B, Vyas S, Li J, Leitner-Dagan Y, Krichevsky A, Taylor T, Vainstein A, Citovsky V., (2005), “pSAT vectors: a modular series of plasmids for autofluorescent protein tagging and expression of multiple genes in plants.” Plant Mol. Biol., 57(4):503-16).

The pSAT vectors contain a plant promoter, an MCS and a plant terminator, which allows for subcloning and expression of one transgene. Cassettes, containing promoter/gene of interest/terminator sequence are derived from pSAT vectors using homing endonucleases and subcloned into the same sites of the pPZP-RCS vector. The pPZP-RCS is a binary vector that includes homing endonuclease enzyme recognition sites in its MCS and allows for cloning of multiple (from 6 or more) pSATs derived cassettes into it, thus serving as a single binary (acceptor) vector. This vector system allows for multiple nuclear transgene expression without requiring bicistronic RNAs or internal ribosome binding sites (IRES). Accordingly, use of pSAT vectors allows introduction of multiple genes into a single acceptor vector. The single pPZP-RCS acceptor vector containing the multiple genes may then be introduced in a single transformation event into a plant, without requiring three or more subsequent plant transformations.

The specific pSATs and GeneBank accession numbers are: pSAT1-EGFP-C1 (SEQ ID NO: 23), pSAT2-EGFP-C1 (SEQ ID NO: 24), pSAT3-EGFP-C1 (SEQ ID NO: 25), pSAT4-EGFP-C1 (SEQ ID NO: 26), pSAT5-EGFP-C1 (SEQ ID NO: 27), pSAT6-EGFP-C1 (SEQ ID NO: 28) and pSAT7-EGFP-C1 (SEQ ID NO: 29), respective NCBI numbers are: AY818363 (SEQ ID NO: 23), AY818365 (SEQ ID NO: 24), AY818366 (SEQ ID NO: 25), AY818367 (SEQ ID NO: 26), AY818368 (SEQ ID NO: 27), AY818377 (SEQ ID NO: 28) and AY818384 (SEQ ID NO: 29).

In another embodiment, the vector is a plastid (chloroplast) transformation vector. Typically, a transgene in a chloroplast transformation vector is flanked by a “homologous recombination site,” which is a DNA region that is homologous to a region of the plastome. The “plastome” refers to the genome of a plastid. The homologous recombination site enables site-specific integration of a transgene expression cassette into the plastome by the process of homologous recombination. Homologous recombination is a process that naturally occurs in plastids. Homologous recombination differs from random transgene integration into plant nuclear genome. An example of a chloroplast transformation vectors arc the pPRV vector series (Lutz K. A., Azhagiri A. K., Tungsuchat-Huang T., Maliga P. (2007) “A guide to choosing vectors for transformation of the plastid genome of higher plants. “Plant Physiol. 145(4):1201-10).

In a preferred embodiment of the invention, the full or partial LUX operon is directly expressed from the chloroplast genome. Insertion of the genes into chloroplast genome is done by cloning the whole LUX operon into a chloroplast transformation vector. Such a method of cloning may include transforming chloroplasts with the vector, and bringing the population of chloroplast genomes copies to homogenicity using standard methods. (Lutz K. A., Svab Z., Maliga P. (2006) “Construction of marker-free transplastomic tobacco using the Cre-loxP site-specific recombination system.” Nat. Protoc. 1(2):900-10).

### Promoters

The heterologous nucleotide sequence or vector described herein may include regulatory sequences useful for expression of a gene product in a host, such a promoter. The term “promoter” refers to a nucleotide sequence capable of controlling the expression of a coding sequence. A promoter drives expression of an operably linked nucleotide sequence. The term “operably linked” as used herein refers to linkage of a promoter to a nucleotide sequence such that the promoter mediates transcription of the nucleotide sequence. A “coding sequence” refers to a nucleotide sequence that encodes a specific amino acid sequence. A promoter is typically located upstream (5′) to a coding sequence.

A wide variety of promoters is known in the art and may be used to facilitate expression of a gene in the heterologous nucleotide sequence. Examples of suitable promoters include constitutive promoters, plant tissue-specific promoters, plant development-specific promoters, inducible promoters, circadian rhythm promoters, viral promoters, male germline-specific promoters, female germline-specific promoters, flower-specific promoters, and vegetative shoot apical meristem-specific promoters.

A “constitutive” promoter refers to a promoter that causes a gene to be expressed in all cell types at all times. An example of a constitutive plastid promoter is psbA, photosystem II reaction center promoter (derived from pCLT146, GeneBank #DQ463359; and rrn, chloroplast 16S rRNA gene promoter (derived from pN-IC101. GeneBank #AY442171).

Examples of nuclear genomic constitutive plant promoters include the cauliflower mosaic virus (CaMV) 35S promoter, which confers constitutive, high-level expression in most plant cells; the nopaline synthase promoter; the octopine synthase promoter; cauliflower mosaic virus 19S promoter; rice actin 1 promoter; manopine synthase promoter; and a histone promoter. Further suitable constitutive promoters include the Rubisco small subunit (SSU) promoter, leguminB promoter, TR dual promoter, ubiquitin promoter, and Super promoter. Different heterologous nucleotide sequences or vectors may contain different promoters to prevent gene silencing when several consecutive genes on a chromosome are expressed from the same promoter.

An “inducible” promoter refers to a promoter that is regulated in response to a stress or stimuli. Examples of inducible promoters include a tetracycline repressor system, Lac repressor system, copper-inducible system, salicylate-inducible system (such as the PR1a system), and alcohol-inducible system. Further examples include inducible promoters that are regulated in response to environmental, hormonal, chemical, and/or developmental stress or stimuli. Such stress or stimuli include heat (e.g., tomato hsp70 promoter or hsp80 promoter); light; hormones (e.g., steroid-inducible MMTV LTR promoter), such as abscisic acid; chemicals, such as methyl jasmonate, salicylic acid; increased salinity; drought; pathogen (e.g. promoter of the PRP1 gene); heavy metals (e.g. heavy metal-inducible metallothionein I promoter and the promoter controlling expression of the tobacco gene cdiGRP; and wounds (e.g. pinII promoter). Preferably, the promoter is a promoter induced by heavy metals.

A “tissue-specific” promoter as used herein refers to a promoter that drives expression of an operably linked nucleotide sequence to a particular tissue. A tissue-specific promoter drives expression of a gene in one or more cell types in a specific organ (such as leaves, or seeds), specific tissues (such as embryo or cotyledon), or specific cell types (such as seed storage cells or leaf parenchyma). Examples include Gentiana triflora promoter for chalcone synthase (NCBI accession AB005484), a seed-specific promoter, such as β-conglycinin, napin promoter, and phaseolin; mature leaves-specific promoter, such as the SAG promoter from Arabidopsis.

Promoters responsible to the circadian rhythm cycle can also be used in the heterologous nucleotide sequence or vector. Such promoters include the native ELF3 promoter and the promoter firom the chlorophyll alb binding protein (CAB2 promoter).

### Prrn Promoter

In one embodiment, the heterologous nucleotide sequence is operably linked to a truncated Prrn promoter. The Prrn promoter is a 16S rRNA operon promoter, typically, a tobacco plastid 16S rRNA operon promoter. An exemplary Prrn promoter is about 150 bp in length. Examples of sequences of a Prrn promoter are shown below:

(SEQ ID NO: 30)
GCTCTAGTTGGATTTGCTCCCCCGCCGTCGTTCAATGAGAATGGATAAGAGGCTCGTGGGAT
TGACGTGAGGGGGCAGGGATGGCTATATTTCTGGGAGCGAACTCCGGGCGAATTTGAAGCGC
TTGGATACAGTTGTAGGGAGGGATCC
(Shimizu et al, “Selectable tolerance to herbicides
by mutated acetolactate synthase genes integrated
into the chloroplast genome of tobacco”, Plant
Physiology, 2008, vol. 147, pp. 1976-1983).
(SEQ ID NO: 31)
TATAGATCCGCTCCCCCGCCGTCGTTCAATGAGAATGGATAAGAGGCTCGTGGGATTGACGT
GAGGGGGCAGGGATGGCTATATTTCTGGGAGTCGAGTAGACCTTGTTGTTGTGAAAATTCTT
AATTCATGAGTTGTAGGGAGGGATTT
(Lutz et al, “Construction of marker-free transplastomic
tobacco using the Cre-loxP site-specific recombination
system”, Nat Prow. 1(2):900-10).

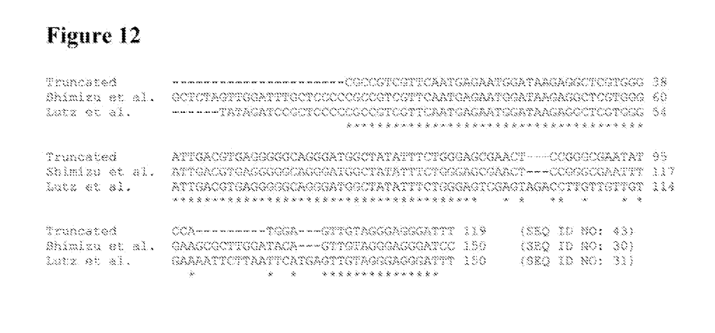

As used herein, a “truncated” Prrn promoter refers to a Prrn promoter that has less nucleotides than the Prrn promoters of SEQ ID NO: 30 and SEQ ID NO: 31. See, for example. FIG. 12. The truncated Prrn promoter may be truncated at the 5′ end and/or the 3′ end, as compared to a Prrn promoter.

In one embodiment, a truncated Prrn promoter is greater than 10 bp in length but less than 150 bp in length. Preferably, the truncated Prrn promoter is between about 80 bp and 100 bp in length. More preferably, the truncated Prrn promoter is between about 90 and 98 bp in length. Most preferably, the truncated Prrn promoter is about 95 bp in length.

Exemplary truncated Prrn promoters include promoters having the following sequences:

| | Base position | |
|---|---|---|
| | difference in | |
| | relation to | |
| Exemplary truncated Prrn promoter | SEQ ID NO: 32 | SEQ ID NO: |
| CGCCGTCGTT CAATGAGAAT GGATAAGAGG | — | SEQ ID NO: 32 |
| CTCGTGGGAT TGACGTGAGG GGGCAGGGAT | | |
| GGCTATATTT CTGGGAGCGA ACTCCGGGCG | | |
| AATAT | | |
| 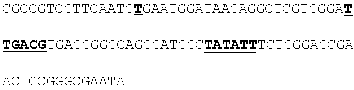 | 16, A → T | SEQ ID NO: 33 |
| 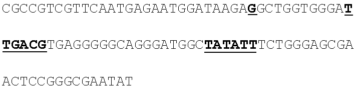 | 33, C → G | SEQ ID NO: 34 |
| 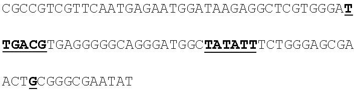 | 84, C → G | SEQ ID NO: 35 |
| 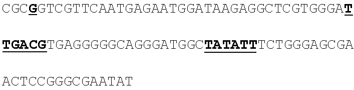 | 4, C → G | SEQ ID NO: 36 |
| 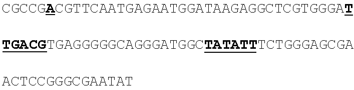 | 6, T → A | SEQ ID NO: 37 |
| 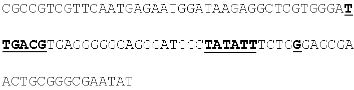 | 74, G → C | SEQ ID NO: 38 |
| 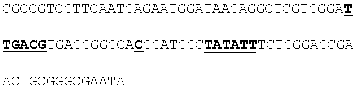 | 56, G → C | SEQ ID NO: 39 |
| 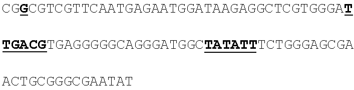 | 3, C → G | SEQ ID NO: 40 |
| 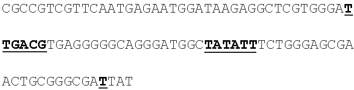 | 92, A → T | SEQ ID NO: 41 |
| 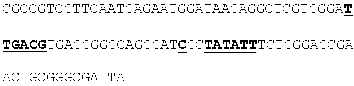 | 61, G → C | SEQ ID NO: 42 |

In one embodiment, the promoter includes a sequence that is at least at least 95% identical to positions 1 to 39, 46 to 63, and 70-95 of the sequence set forth in SEQ ID NO: X, wherein said promoter has 100% identity to positions 40-45 of the sequence set forth in SEQ ID NO: X. For example, the promoter may have at least one substitution at any one of the following positions: 3, 4, 6, 16, 33, 84, 74, 56, 92, or 61.

In another embodiment, the promoter includes a sequence that is at least at least 98% identical to positions 1 to 39, 46 to 63, and 70-95 of the sequence set forth in SEQ ID NO: X.

In yet another embodiment, the promoter includes a sequence that is at least at least 99% identical to positions 1 to 39, 46 to 63, and 70-95 of the sequence set forth in SEQ ID NO: X.

The exemplary truncated Prrn promoter preferably includes a conserved region. The term “conserved region” or “conserved domain” as used herein refers to a region conserved in prokaryotic and plastidal promoters, namely the −10 TATA region and −35 element. For example, the conserved region includes a relatively high degree of sequence identity (about 98% to 100%) exists between the distinct sequences. In one embodiment, the conserved region of the truncated Prrn promoter is at positions 40-45 and/or positions 64-69 of the sequence set forth in SEQ ID NO: 32.

In another embodiment, the truncated Prrn promoter includes a transcriptional leader sequence. In an exemplary embodiment, the truncated Prrn promoter further includes a restriction site, such as, for example, a NcoI site, to fuse the leader sequence to the promoter. In a preferred embodiment, the truncated Prrn promoter including a leader sequence (in italics) and Nco I site (CCATGG) has a sequence as shown:

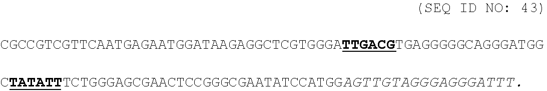

### Leader and Terminator Sequences

The heterologous nucleotide sequence or vector may also include leader sequences, such as; rbcL, ribulose-bisphosphate carboxylase gene leader sequence (derived from pCLT516, GeneBank #DQ882177: (SEQ ID NO: 44); and Shine-Dalgarno consensus ribosome binding sequence (AGGAGG); and terminators, such as psbA, which is a photosystem II reaction center terminator (derived from pCLT146, GeneBank #DQ463359: (SEQ ID NO: 45); and rps16 gene rps16 terminator (derived from pL3 vector series, GeneBank #EU520589, EU520588, EU520587: (SEQ ID NO: 46). Another exemplary terminatory is a Cauliflower mosaic virus (CaMV) 35S terminator.

### Marker

In addition, the heterologous nucleotide sequence or vector may include a nucleotide sequence for a selectable and/or screenable marker. A “selection marker” refers to a protein necessary for survival or growth of a transformed plant cell grown in a selective culture regimen. Typical selection markers include sequences that encode proteins, which confer resistance to selective agents, such as antibiotics, herbicides, or other toxins. Examples of selection markers include genes for conferring resistance to antibiotics, such as spectinomycin, streptomycin, tetracycline, ampicillin, kanamycin, G 418, neomycin, bleomycin, hygromycin, methotrexate, dicamba, glufosinate, or glyphosate.

Various other selection markers confer a growth-related advantage to the transformed cells over the non-transformed cells. Examples include selection markers for β-glucuronidase (in conjunction with, for example, cytokinin glucuronide), mannose-6-phosphate isomerase (in conjunction with mannose), and UDP-galactose 4-epimerase (in conjunction with, for example, galactose).

Selection markers include those which confer resistance to spectinomycin (e.g. encoded by the resistance gene, aadA), streptomycin, kanamycin, lincomycin, gentamycin, hygromycin, methotrexate, bleomycin, phleomycin, blasticidin, sulfonamide, phosphinothricin, chlorsulfuron, bromoxynil, glyphosate, 2,4-D, atrazine, 4-methyltryptophan, nitrate, S-aminoethyl-L-cysteine, lysine/threonine, aminoethyl-cysteine or betaine aldehyde. Preferably, the selection marker is functional in plastids. Especially preferred are the genes aadA (GeneBank NC_{—}009838). nptII (GeneBank FM177583). BADH (GeneBank AY050316), aphA-6 (GeneBank X07753).

After a heterologous nucleotide sequence has been introduced into a host cell, it may be advantageous to remove or delete certain sequences from the plastome or genome of the plant or cell. For example, it may be advantageous to remove a selection marker gene that has been introduced into a genome if the selection marker is no longer necessarily required after the selection phase. Methods for directed deletion of sequences are known in the art. For example, the nucleotide sequence encoding a selection marker preferably includes a homology-based excision element, such as Cre-lox and attB/attP recognition sequences, which allow removal of the selection marker genes using site-specific recombinases.

In one embodiment, the heterologous nucleotide sequence or vector includes reporter genes. Reporter genes encode readily quantifiable proteins which, via their color or enzyme activity, allow an assessment of the transformation efficiency, the site or time of expression or the identification of transgenic plants. Examples of reporter genes include green fluorescent protein (GFP), luciferase, β-Galactosidase, β-Glucuronidase (GUS), R-Locus gene product, β-Lactamase, xylE gene product, alpha-amylase, and tyrosinase.

The heterologous nucleotide sequence or vector may include sequences encoding a fluorescent protein that are excited or fluoresce at different wavelengths, at different periods of time, or under different conditions. Example of such fluorescent protein is DsRed (GeneBank #EU827527, DsRed-Monomer gene, synthetic construct) (SEQ ID NO: 47), which can fluoresce and emit light at red wavelengths, or GFP, which can fluoresce and emit light at green wavelengths.

### Functional Elements

The heterologous nucleotide sequence or vector may also include functional elements, which influence the generation, multiplication, function, use or value of the heterologous nucleotide sequence or vector used within the scope of the present invention. Examples of functional elements include replication origins (ORI), which make possible an amplification of the heterologous nucleotide sequence or vector according to the invention in, for example, E. coli or in plastids; multiple cloning sites (MCSs), which permit and facilitate the insertion of one or more nucleic acid sequences; homologous recombination sites, allowing stable recombination of transgenes into plastid genome; and border sequences, which make possible Agrobacterium-mediated transfer of the heterologous nucleotide sequence or vector into plant cells for the transfer and integration into the plant genome, such as, for example, the right or left border of the T-DNA or the vir region.

The heterologous nucleotide sequence or vector may optionally include RNA processing signals, e.g. introns, which may be positioned upstream or downstream or within a polypeptide-encoding sequence in the heterologous nucleotide sequence. Intron sequences are known in the art to aid in the expression of heterologous nucleotide sequences in plant cells.

### Cofactors

In another embodiment, the heterologous nucleotide sequence or vector includes at least one gene encoding a cofactor for enhancing autoluminescence. As used herein, the term “cofactor” refers to an organic molecule, an inorganic molecule, a peptide, or a protein required for enzyme activity. The protein products encoded by the LUX genes may require the cofactors for regenerating and enhancing FMNH₂ pool, and fatty acid precursors in order to induce autoluminescence.

In some applications of the present invention, the level of luminescence may be enhanced by introduction of a genes involved in riboflavin biosynthesis (i.e. RIB operon or a flavin reductase) and/or genes encoding for fatty acid donors (i.e. genes belonging to the Fatty Acids Synthase [either FASI or FASII] pathway). In particular, in some embodiments, a component of the RIB operon (such as, for example, ribE and ribH genes (encoding riboflavin synthase or lumasine synthase, respectively), or the RIB operon as a whole) involved in riboflavin synthesis and/or donors of fatty acids for the aldehyde synthesis, such as bacterial or plant acyl carrier protein (ACP), can be transfected into a plant cell as part of a heterologous nucleotide sequence or vector. In plants, ACP exists as a small cofactor protein that participates in reactions of fatty acid biosynthesis and metabolism. Also, a flavin reducatase enzyme, such as Fre from E. coli or Frp from Vibrio harveyi, can be introduced to increase FMNH₂ turnover.

Specific examples of suitable cofactors for enhancing autoluminescence include polypeptides encoded by the RIB operon (GeneBank accession AF364106) (SEQ ID NO: 48), bacterial acyl carrier protein, plant acyl carrier protein, transcriptional activators, and FRE flavin reductases enzymes from either luminescent (P. luminescens (GeneBank #D17745) (SEQ ID NO: 49) and V fischeri (GeneBank #D17744) (SEQ ID NO: 50), or Vibrio harveyi FRP (GeneBank #VHU08996) (SEQ ID NO: 54), or other bacteria (E. coli FRE, GeneBank #NC_{—}010473) (SEQ ID NO: 51). Further examples of suitable cofactors include riboflavin kinases (RFK) such as plant Arabidopsis thaliana RFK (GeneBank #NC_{—}003075) (SEQ ID NO: 52) or bacterial E. coli RFK (GeneBank #NC_{—}009801) (SEQ ID NO: 53).

As used herein, “enhancing” autoluminescence refers to increased autoluminescent intensity or brightness that is greater than that without the cofactor. Enhancing autoluminescence may further include replenishing exhausted luciferin or other substrate or cofactor or other protein in order to continue or revive the reaction for autoluminescence.

The term “RIB operon” refers to an operon containing genes coding for proteins essential to production of riboflavin. The RIB operon in the bacteria belonging to the genus Bacillus includes following genes: ribO gene coding for control element, ribG gene coding for deaminase/reductase, ribB gene coding for riboflavin synthase (a-subunit), ribA gene coding for GTP-cyclohydrolase/3,4-dihydroxy-2-butanon-4-phosphate synthase, ribH gene coding for lumasine synthetase, and ribT gene coding for a protein with unknown function. Nucleotide sequences of ribG, ribB, ribA, ribH and ribH genes of Bacillus subtilis are presented in GenBank under accession numbers X51510 (B. subtilis riboflavin biosynthesis operon ribG, ribB, ribA, ribH, and ribT genes) (SEQ ID NO: 55). The rib genes for Escherichia coli include rib, ribA, and ribE code for GTP cyclohydrolase II, 3,4-dihydroxy-2-butanone 4-phosphate (DHBP) synthetase, and riboflavin synthetase, respectively. Nucleotide sequences of rib, ribA, and ribE genes of E. coli are presented in EBI under accession numbers ABV17158 (SEQ ID NO: 56) and CAA48861 (SEQ ID NO: 57), respectively. Similarly, Photobacterium leiognathi, strain PL741, RIB operon, encoding for rib E, H, B and A genes can be found at the GeneBank under accession number AF364106 (SEQ ID NO: 58).

The term “plant acyl carrier protein” or “bacterial acyl carrier protein” refers to any acyl carrier protein having the essential functional characteristics of naturally occurring ACP molecules found in plants or bacteria, respectively. Nucleotide sequences encoding a plant or bacterial acyl carrier protein include those presented in GenBank such as Arabhidopsis thaliana ACP (EBI#X13708) (SEQ ID NO: 59) and Photobacterium sp. ACP (EBI #: EAR53459) (SEQ ID NO: 60).

Increased and/or Modified Luminescence

In another embodiment, autoluminescence levels can be augmented by an increase of activity of enzymes involved in the light emission reaction. For example, the LUX operon or the luciferase can be expressed under a strong promoter, thereby allowing increase in concentration of the LUX operon proteins within a given cell and thus higher light output, as compared to a cell without a strong promoter.

Additional exemplary methods to increase luciferase and/or other proteins coded by the LUX operon, include directed evolution, protein engineering and rational design. For example, directed evolution is a known tool in the art that can be used to significantly improve enzyme activity, selectivity, stability and other parameters, as compared to an identical naturally occurring enzyme that has not undergone directed evolution. For example, application of direct evolution methodology to glyphosate N-acetyltransferase (GAT) resulted in a variant with a 10,000-fold improvement in catalytic efficiency, compared with that of the parental enzyme; another example is Renilla luciferase, which was modified to be 200-fold more resistant to inactivation and produce 4-fold higher light output then the parental luciferase. Further exemplary methods include codon optimization, as known in the art, and/or use of diverse ribosome binding sites to enhance expression of a particular gene, or coordinate gene expression, within the plastid.

In another embodiment, wavelength (color) of the emitted light can be modified. The color of the light emitted by the plant-expressed bacterial luciferase can be changed and modified by either of the two following exemplary approaches: (i) change in luciferase properties using direct evolution and protein engineering, as is known in the art to change enzymatic properties of different luciferases, or (ii) coupling with an appropriate chromophore. For example, Enhanced Green Fluorescent Protein (EGFP) has an excitation peak at about 490 nm, and emission peak at about 510 nm. Coupling of the bacterial luciferase (emitting at about 490 nm) with EGFP will allow to further shift the luminescence into different emission spectra and prevent pigment interference in a given tissue. Another example is the LuxY-encoded Yellow Fluorescence Protein (YFP) from certain V. fischeri strains. The YFP causes a shift in the luminescence from about 490 nm to a higher wavelength, resulting in the emission of a yellow, rather than a blue-green light. Shift in light emission will be instrumental for both generation of multiple varieties of the same ornamental plant product, differing in color of the emitted light, as well as for decrease absorption of the luciferase emitted light by plant pigments by shifting emission peak away from pigment's absorption peaks.

In yet another embodiment, the autoluminescent plants are rendered sterile and incapable of reproduction. For example, the heterologous nucleotide sequence may include a sterility operon, which refers to one or more genes rendering the plant incapable of reproduction. Sterility operons are known in the art.

In other embodiment, the heterologous nucleotide sequence includes a toxin encoding sequence operably linked to a plant-embryo specific promoter. Production of the toxin in the developing plant embryos will lead to cell death within those embryos, thus terminating their development and leaving the plant sterile.

### Vector System

In another aspect, the invention relates to a vector system. The vector system includes a first heterologous nucleotide sequence includes a plastid transformation vector having a first heterologous nucleotide sequence. The first heterologous nucleotide sequence includes a bacterial LUX operon, which includes LUX A, LUX B, LUX C, LUX D, LUX E, and LUX G genes, wherein the heterologous nucleotide sequence is operably linked to a first promoter, and wherein the heterologous nucleotide sequence is capable of being incorporated into a plastid genome. The vector system further includes a vector having a second heterologous nucleotide sequence operably linked to a second promoter.

In one embodiment, the first promoter is a truncated Prrn promoter, as described above.

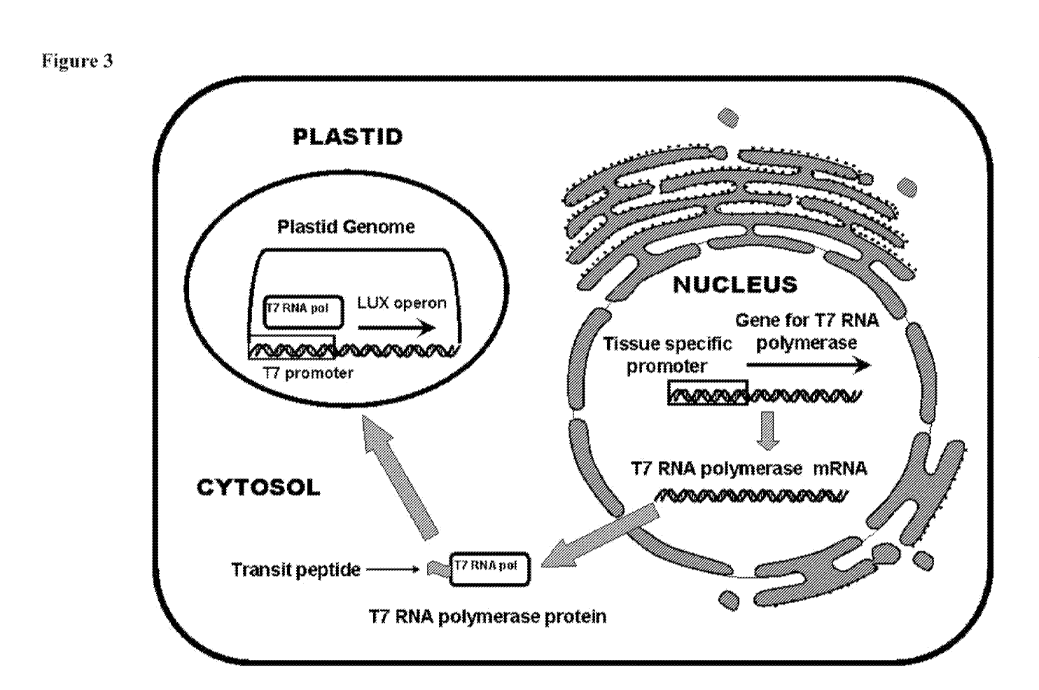

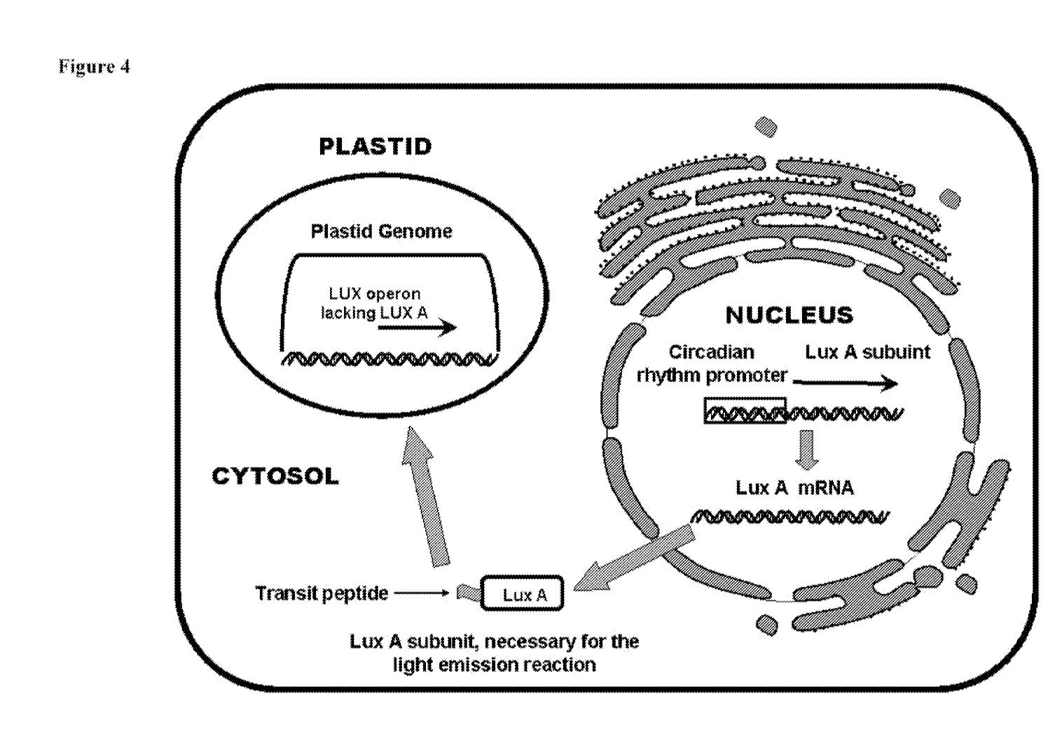

In another embodiment, the first promoter is an inducible promoter that is inducible by a protein encoded by the second heterologous nucleotide sequence. For example, a first heterologous nucleotide sequence includes a LUX operon and an inducible promoter. A second heterologous nucleotide sequence includes a promoter and a gene encoding a transcription factor. The transcription factor induces the inducible promoter, thereby activating transcription of the LUX operon genes. See FIGS. 3 and 4.

The term “transcription factor” refers to any protein that is involved in the initiation of transcription. In this embodiment, it might not be, or it might be an RNA polymerase, as in the case of T7 DNA polymerase directly activating a promoter (see FIG. 3) Transcription factors interact preferentially with specific nucleotide sequences. i.e., regulatory sequences, and which in appropriate conditions stimulate transcription (“transcriptional activator”) or repress transcription (“transcriptional repressor”).

In yet another embodiment, the first promoter is a constitutive promoter and the second heterologous nucleotide sequence further includes a plastid targeting sequence.

For example, the promoter for the first heterologous nucleotide sequence is inducible by a transcription factor in order to activate transcription of the LUX operon. An exemplary promoter is a T7 promoter (for example, SEQ ID NO: 61), which is inducible by T7 RNA polymerase (for example, SEQ ID NO: 62) (FIG. 3).

In one embodiment, the promoter for the second heterologous nucleotide sequence is an inducible promoter, such as a heavy metal sensitive promoter from tobacco cdiGRP gene, or a tissue-specific promoter.

An exemplary second heterologous nucleotide sequence further includes a plastid targeting sequence and/or a reporter gene. See FIGS. 3 and 4. For example, a first heterologous nucleotide sequence includes a LUX operon and an inducible promoter, such as the T7 promoter. A second heterologous nucleotide sequence includes a tissue-specific promoter or circadian rhythm promoter or otherwise inducible (stress, heavy metal, etc) promoter in the nucleus. The second heterologous nucleotide sequence further encodes a T7 RNA polymerase. Accordingly, when the second promoter is activated, the gene for the T7 RNA polymerase will be transcribed and then targeted to a plastid (e.g., a chloroplast) due to the N-terminally fused plastid transit peptide. The LUX genes in the chloroplast will be driven by the T7 promoter, to which T7 RNA polymerase binds and thus activates LUX transcription. Thus, activation of the LUX operon is indirect. (FIG. 3).

In yet another aspect, the invention relates to a vector system. The vector system includes a plastid transformation vector having a first heterologous nucleotide sequence. The first heterologous nucleotide sequence includes any five of the following LUX A, LUX B, LUX C, LUX D, LUX E, and LUX G genes, wherein the heterologous nucleotide sequence is operably linked to a truncated Prrn promoter, and wherein the heterologous nucleotide sequence is capable of being incorporated into a plastid genome. The vector system further includes a vector having a second heterologous nucleotide sequence that includes plastid targeting sequence and the sixth LUX gene operably linked to a second promoter (FIG. 4).

For example, in one embodiment, first heterologous nucleotide sequence includes LUX B, LUX C, LUX D, LUX E, and LUX G genes, and the second heterologous nucleotide sequence includes LUX A gene. The LUX A gene is expressed from an inducible promoter in the nucleus and targeted into the plastid using transit peptide. While rest of the genetic machinery required for the luminescence is constantly expressed in the plastid, for instance driven by the truncated Prrn promoter, light emission will occur when the light emission machinery is complemented by the LUX A subunit targeted from the nucleus, which in turn is regulated by an inducible promoter. See FIG. 4.

### Kit

In another aspect of the invention, a kit is provided. The kit includes a seed for generating a transgenic autoluminescent plant cell having a heterologous nucleotide sequence which includes a bacterial LUX operon, which includes LUX A, LUX B, LUX C, LUX D, LUX E, and LUX G genes, wherein the heterologous nucleotide sequence is operably linked to a truncated Prrn promoter, and wherein the heterologous nucleotide sequence is integrated in a plastid genome. The kit also includes a plant transformation vector as described above.

The kit can further include reagents, buffers, and materials related to any of the nucleotide sequences and proteins described above. In addition, the kit can include a plant or plant cell produced by the invention.

### Variants

The present invention further relates to variants of the nucleotide sequences described herein. Variants may occur naturally, such as a natural allelic variant. Other variants include those produced by nucleotide substitutions, deletions, or additions. The substitutions, deletions, or additions may involve one or more nucleotides. These variants may be altered in coding regions, non-coding regions, or both. Alterations in the coding regions may produce conservative or non-conservative amino acid substitutions, deletions, or additions. Preferably, the variant is a silent substitution, addition, or deletion, which does not alter the properties and activities of the peptide encoded by the nucleotide sequence described herein. Conservative substitutions are also preferred.

Further embodiments of the invention include variant nucleotide sequences comprising a sequence having at least 90% identical, and more preferably at least 95%, 96%, 97%, 98%, or 99% identical to a nucleotide sequence described herein. The nucleotide sequences described herein are the “reference” sequences.

For example, a variant nucleotide sequence that is at least 95% identical to a reference nucleotide sequence (e.g., the LUX operon) described herein is identical to sequence described herein except that the variant nucleotide sequence may include up to five point mutations per each 100 nucleotides of the reference nucleotide sequence (e.g., the LUX operon) sequence described herein.

In other words, to obtain a variant nucleotide sequence that is at least 95% identical to a reference nucleotide sequence described herein, up to 5% of the nucleotides in the reference sequence may be deleted or substituted with another nucleotide, or a number of nucleotides up to 5% of the total nucleotides in the reference sequence may be inserted into the reference sequence.

These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The term “sufficiently identical” as used herein refers to a first nucleotide sequence that contains a sufficient or minimum number of identical or equivalent nucleotides to a second nucleotide sequence, such that the first and second nucleotide sequences share common structural domains or motifs and/or a common functional activity. For example, nucleotide sequences that share common structural domains having at least 80%, 85%, 90%, 91%, 92%, 93%, 94%. 95%, 96%, 97%, 98%, 99%, or more identity across the sequences, and share a common functional activity are defined herein as sufficiently identical.

To determine percent identity of two nucleic acid sequences, the sequences are aligned for optimal comparison purposes (e.g. gaps can be introduced in one or both of a first and second nucleotide sequence for optimal alignment). For example, when aligning a first sequence to a second sequence having 10 nucleotides, at least 70%, preferably at least 80%, more preferably at least 90% of the 10 nucleotides between the first and second sequences are aligned. When a position in the first sequence is occupied by the same nucleotide as the corresponding position in the second sequence, then the molecules are identical at that position. The percent identity between the two sequences is a function of the number of identical positions shared by the sequences, taking into account the number of gaps, the length of the sequences, and the length of each gap that need to be introduced for optimal alignment of the two sequences. An algorithm known in the art may be used to determine percent identity between two sequences.

### INCORPORATION OF SEQUENCE LISTING

Incorporated herein by reference in its entirety is the Sequence Listing for the application. The Sequence Listing is disclosed on a computer-readable ASCII text file titled, “sequence_listing1795-3PCT.txt”, created on Feb. 25, 2010. The sequence listing text file is 258 kb in size.

### EXAMPLES

### Example 1

### Construction of Chloroplast Transformation Vectors

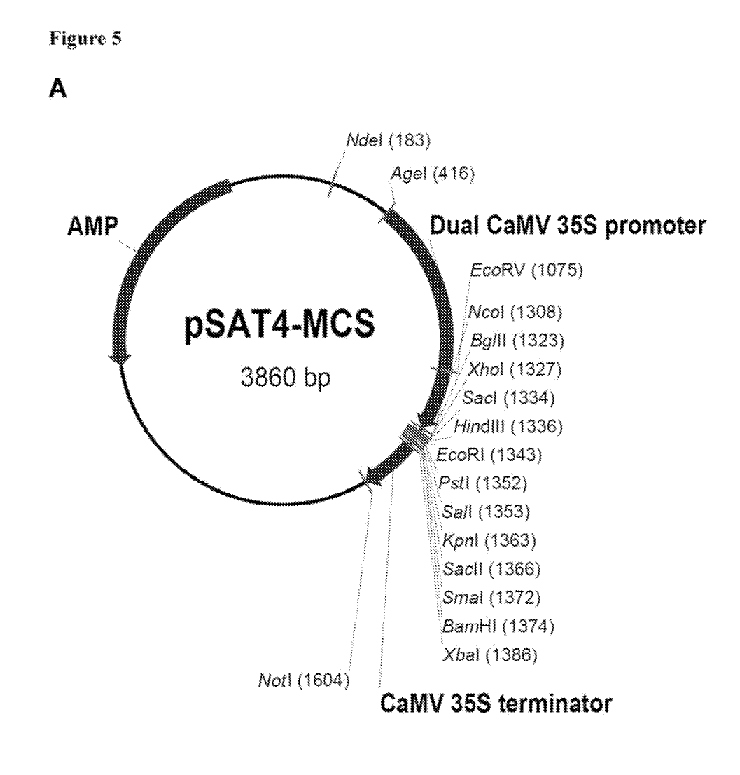

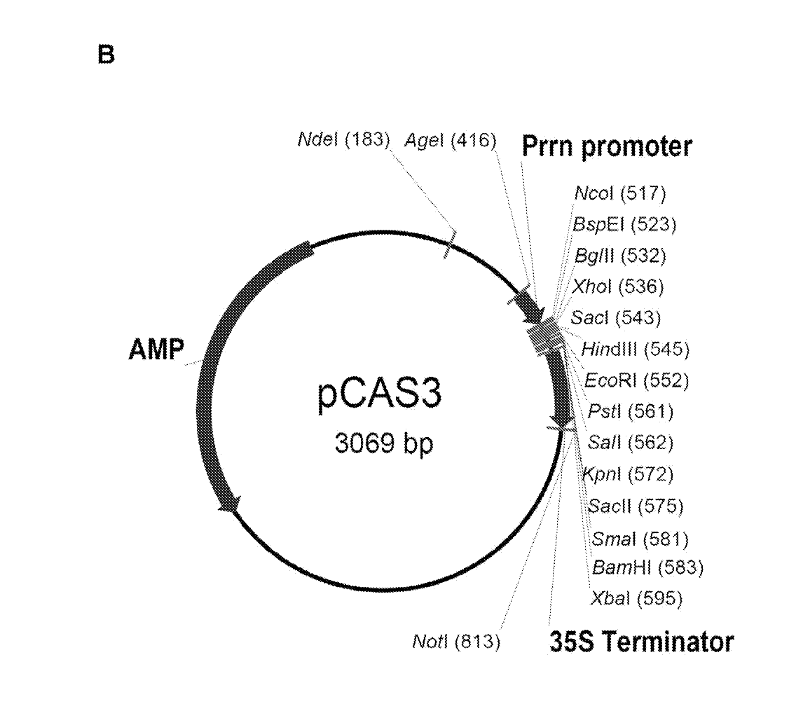

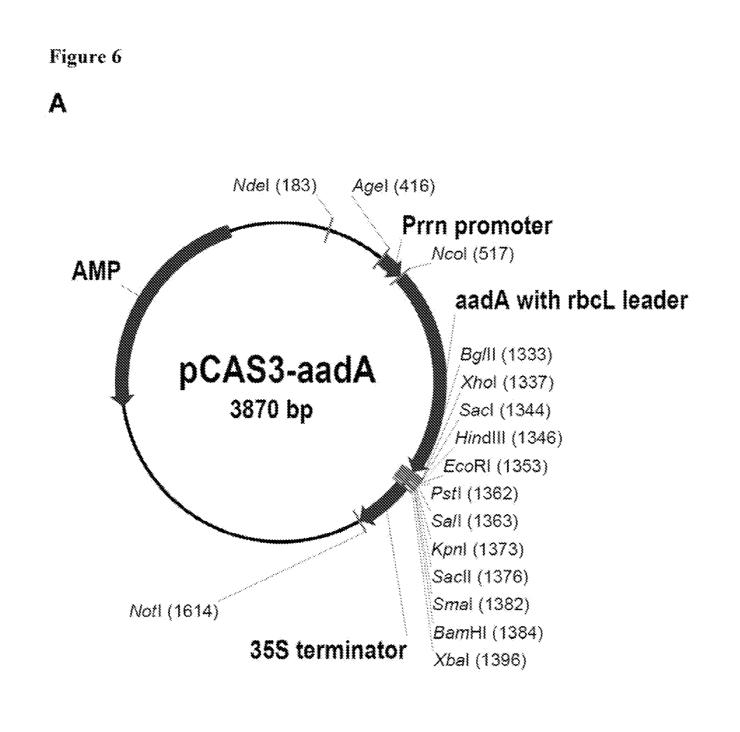

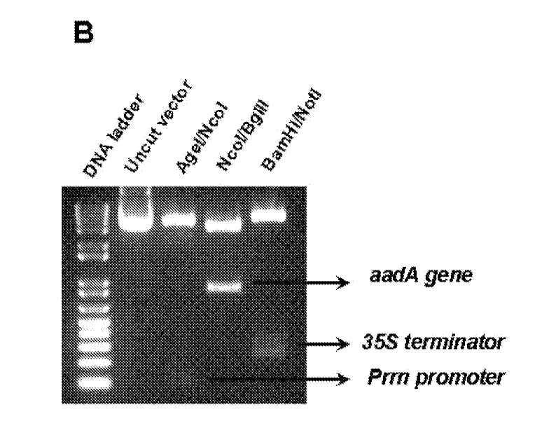

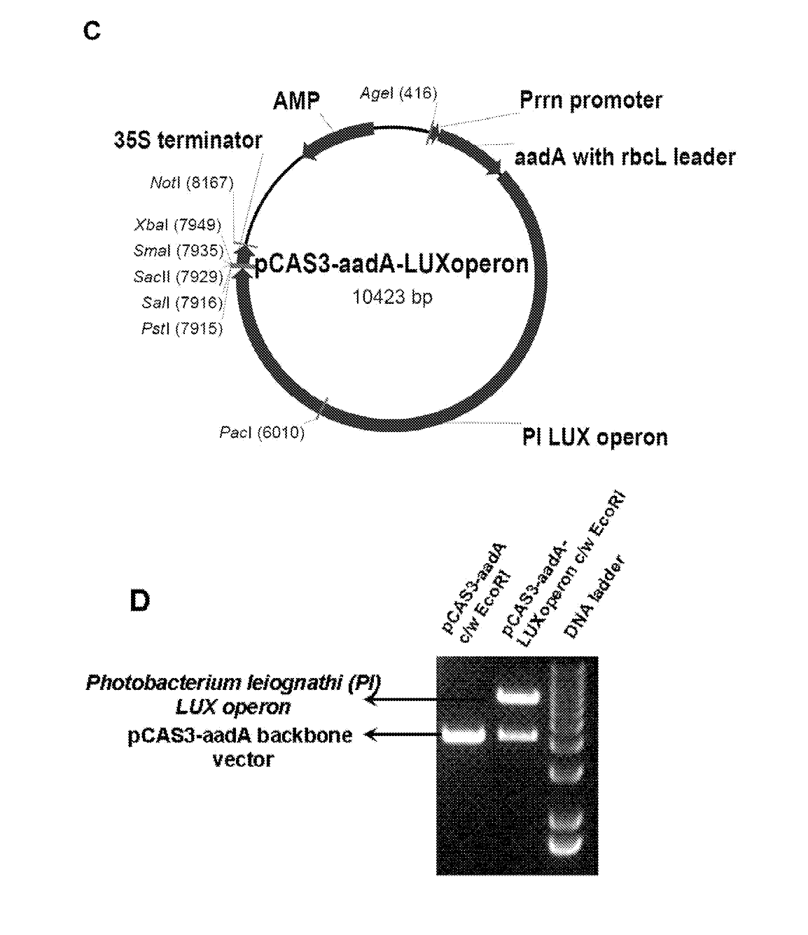

The chloroplast transformation vectors of the pCAS series have been constructed using the backbone of pSAT4-MCS vector (GenBank: DQ005466.1, FIG. 5A and SEQ ID NO: 63 in sequence listing). Please note, any other vector from the pSAT series (Tzfira T, Tian G W, Lacroix B, Vyas S, Li J, Leitner-Dagan Y, Krichevsky A, Taylor T, Vainstein A, Citovsky V. (2005), “pSAT vectors: a modular series of plasmids for autofluorescent protein tagging and expression of multiple genes in plants.” Plant Mol. Biol., 57(4):503-16), and potentially any DNA vector used for routine cloning purposes (such as pUC18 or pUC19 [Fermentas]), can be used as a backbone for the pCAS chloroplast transformation vectors. The eukaryotic 35S CaMV promoter of pSAT4-MCS has been replaced by a truncated version of chloroplast Prrn promoter (SEQ ID NO: 64). Prrn has been cloned as AgeI/NcoI PCR fragment amplified using forward 5′-TCACCGGTCGCCGTCGTTCAATGAGAATGG-3′ (SEQ ID NO: 76) and reverse 5′-GAGCGAACTCCGGGCGAATATCCATGGTT-3′ (SEQ ID NO: 77) primers and Nicotiana tabacum (tobacco) plastid genomic DNA as a template. The CaMV 35S terminator sequence (35ST) has been left as is, since terminator sequences have shown to be, in many instances, superfluous for chloroplast transgene expression. The resulting vector has been designated as pCAS3 (FIG. 5B). A spectinomycin resistance gene aadA (SEQ ID NO: 65), fused to an rbcL leader sequence (SEQ ID NO: 66), has been cloned into pCAS3 as BglII/NcoI PCR fragment amplified using forward 5′-AACCATGGAGTTGTAGGGAGGGATTTATGGGGGAAGCGGTGATCGCC-3′ (SEQ ID NO: 78) and reverse 5′-TGGAGATCTfIATT GCCGACTACCTTGGTGATC-3′ (SEQ ID NO: 79) primers and cloning vector pPZP-RCS2 as a template. Please note, that essentially any other chloroplast transformation vector containing aadA gene sequence may be used as a template for PCR reaction producing aadA for the pCAS vectors. The resulting vector has been designated as pCAS3-aadA (SEQ ID NO: 67 and FIG. 6A). Actual restriction digest demonstrating presence of all cloned genetic elements in pCAS3-aadA vector is shown in FIG. 6B.

Next, The LUX operon (SEQ ID NO: 68, based on GenBank #M63594) from Photobacterium leiognathi (ATCC 25521), comprising LUX genes CDABEG, has been cloned as EcoRI PCR fragment amplified using forward 5′-ACAGAATTCCCAAAGGAGATTACATGATTAAG-3′ (SEQ ID NO: 80) and reverse 5′-TTGGAATTCTTACGTATAGCTAAATGCATCAG-3′ (SEQ ID NO: 81) primers and Photobacterium leiognathi genomic DNA as a template, into the same sites of pCAS3-aadA. Directionality of the cloned LUX operon has been determined using directional restriction digest (such as PacI/SacII) and sequencing. The resulting vector carrying Photobacterium leiognathi (Pl) LUX operon has been designated as pCAS3-aadA-LUXoperon (SEQ ID NO: 69 and FIG. 6C). Actual restriction digest demonstrating presence of LUX operon within the pCAS3-aadA vector is shown in FIG. 6D.

The LUX operon was intended to be introduced into two loci within the chloroplast genome, varying by their read-through transcriptional activity, the rps12/TrnV locus and, relatively more transcriptionally active, TrnI/TrnA locus. To make the pCAS3-aadA-LUXoperon vector suitable for integration into the aforementioned loci, homologues recombination (HR) sequences have been cloned to flank the LUX operon expression cassette. All of the HR sequences required for LUX operon insertion into rps12/TrnV and TrnI/TrnA loci were PCR amplified from Nicotiana tabacum (tobacco) plastid genomic DNA template and then cloned into pCAS3-aadA-LUXoperon vector. Specifically, for targeting integration of the LUX operon into the rps12/TrnV locus, the rps12 homologues recombination sequence (SEQ ID NO: 70) has been cloned into pCAS3-aadA-LUXoperon vector as AgeI PCR fragment amplified using forward 5′-AGTTAGAACCGGTGAAGTGCTTCGAATCATTGCTAITTG-3′ (SEQ ID NO: 82) and reverse 5′-CGATCTAACCGGTTTATCAACTGCCCCTATCGGAAATAGG-3′ (SEQ ID NO: 83) primers.

Due to technical difficulty in cloning of PCR fragments directly into a large-sized plasmids, such as pCAS3-aadA-LUXoperon (>10 Kbp), we have employed several specialized cloning techniques. First, we used an intermediate-step cloning, where we initially sub-cloned an HR sequence PCR fragment, such as rps12, into a smaller sized pSAT4-MCS vector (<4.0 Kbp), then excised it using appropriate enzyme (i.e. AgeI for rps12) and only then cloned it into the same sites of pCAS3-aadA-LUXoperon. Furthermore, in some instances we employed restriction enzyme inactivation technique, avoiding resolution of the digested large DNA backbone vector on an agarose gel. Cloning of rps12 HR sequence into pCAS3-aadA-LUXoperon backbone vector can be demonstrated as an example of this method. First we would excise the DNA insert from the intermediate cloning vector. i.e. cutting out rps12 HR sequence from pSAT4-MCS using AgeI, resolve the fragments on the agarose gel and clean out the rps12 insert fragment using Gel DNA Recovery Kit (Zymogen). Then we would fully digest the backbone pCAS3-aadA-LUXoperon vector with an appropriate enzyme—AgeI in case of rps12 cloning—and proceed to heat inactivation of the AgeI enzyme according to the manufacturer instructions.

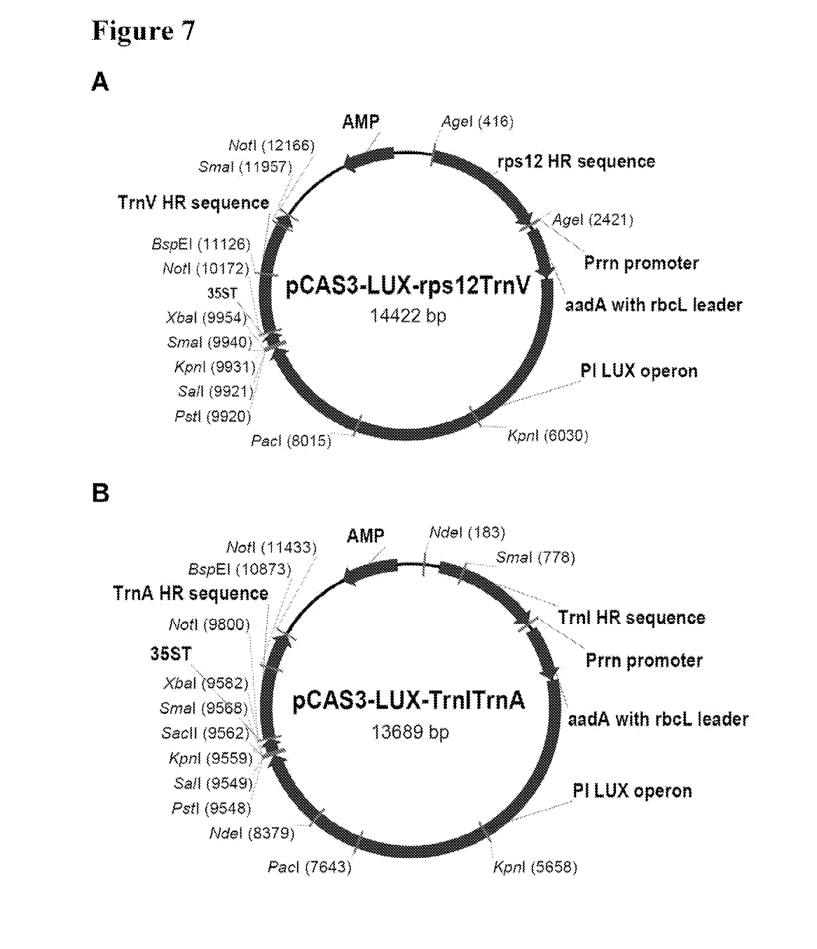

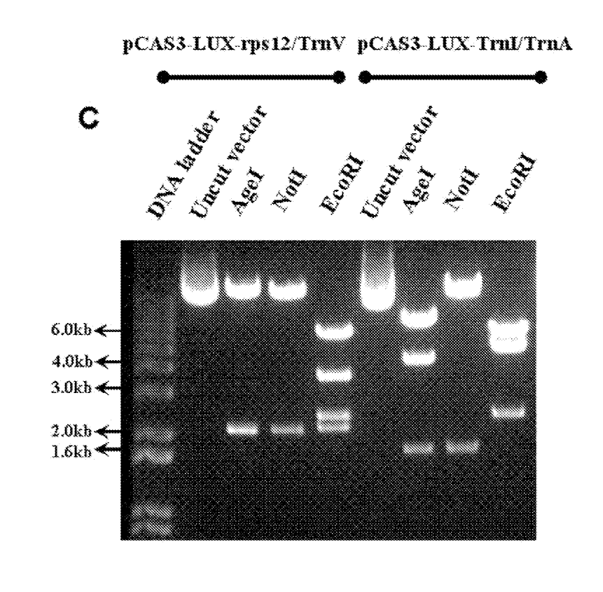

Following enzymatic restriction, the fully digested backbone vector was treated with Antarctic Phosphatase enzyme (AP, New England Biolabs), to prevent vector self ligation in later cloning steps, and the AP enzyme was also heat inactivated according to manufacturer's instructions. An aliquot of the digested and dephosphorilated backbone pCAS3-aadA-LUXoperon was mixed with previously gel-purified rps12 HR insert DNA, and the two fragments have been ligated using T4 DNA Ligase (New England Biolabs) according to manufacturer's instructions. The ligation products have been transformed into XL10-Gold competent cells (Stratagene), suitable for transformation of large DNA molecules with high efficiency. Finally, the directionality of the insert, such as rps12, has been verified using directional restriction digest and sequencing. Please note that other HR sequences, as well as other DNA inserts mentioned herein and introduced into pCAS3-aadA-LUXoperon backbone, frequently have been cloned in a similar manner. The TrnV HR sequence (SEQ ID NO: 71), similarly to rps12 homologues recombination site, has been PCR amplified using forward 5′-ATAATGCGGCCGCCAATTGAATCCGATTTTGACCATTATTTTC-3′ (SEQ ID NO: 84) and reverse 5′-ATTATGCGGCCGCGTGAAGCAGTGTCAAACCAAAATACC-3′ (SEQ ID NO: 85) primers and cloned into the NotI site of pCAS3-aadA-LUXoperon vector already containing the rps12 homologues recombination sequence. Directionality of the cloned TrnV HR fragment has been determined using directional restriction digest and sequencing. The resulting vector has been designated as pCA3-LUX-rps12/TrnV (SEQ ID NO: 72 and FIG. 7A).

To integrate LUX operon into the TrnI/TrnA locus of the chloroplast genome, the TrnI/TrnA HR sequences had to be cloned into the pCAS3-aadA-LUXoperon vector. The TrnI DNA fragment was required to be cloned first since TrnA HR sequence contains AgeI recognition sequence. The TrnI HR sequence (SEQ ID NO: 73) has been PCR amplified using forward 5′-AGTTAGAACCGGTCTTCGGGAACGCGGACACAGGTGG-3′ (SEQ ID NO: 86) and reverse 5′-CGATCTAACCGGTAGATGCTTCTTCTATTCTTTTCCCTG-3′ (SEQ ID NO: 87) primers and cloned using AgeI into the same site of pCAS3-aadA-LUXoperon vector. The TrnA DNA fragment (SEQ ID NO: 74) has been PCR amplified using forward 5′-CTATTATGCGGCCGCACTACTTCATGCATGCTCCACTTGG-3′ (SEQ ID NO: 88) and reverse 5′-GAATGATGCGGCCGCCCTATGAAGACTCGCTTTCGCTACG-3′ (SEQ ID NO: 89) primers and cloned using NotI into the same site of pCAS3-aadA-LUXoperon vector containing the TrnI HR sequence. Directionality of the cloned HR sequences has been determined using directional restriction digest and sequencing. The resulting vector has been designated as pCA3-LUX-TrnI/TrnA (SEQ ID NO: 75 and FIG. 7B). Actual restriction digest demonstrating presence of the cloned HR sequences within the pCA3-LUX-rps12/TrnV and pCA3-LUX-TrnI/TrnA vectors is shown in FIG. 7C. Please note that all constructed vectors have been verified by sequencing.

### Example 2

### Assessment of pCAS-3 LUX Vector Workability in E. coli

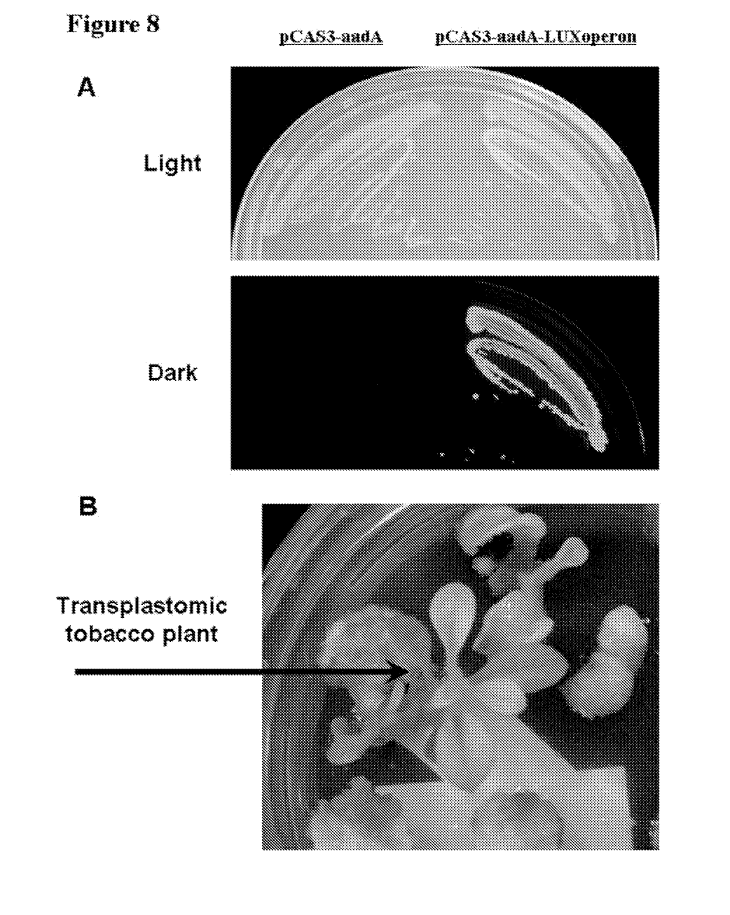

Prior to generation of transplastomic plants, the workability of various pCAS3 vectors has been assessed in E. coli. High functional similarity of promoters and other genetic elements between bacteria and plastids permits, in many instances, expression of plastidal expression cassettes in bacteria. As shown in FIG. 8A (upper panel), pCAS3-aadA and pCAS3-aadA-LUXoperon vectors conferred growth of DH5α E. coli cells on LB medium supplemented with 50-100 μg/ml of spectinomycin, due to expression of the antibiotic resistance aadA gene driven by the plastidal truncated Prrn promoter. Moreover, DH5α E. coli cells harboring pCAS3-aadA-LUXoperon vectors emitted visible light (FIG. 8A, lower panel), due to expression of the LUX operon expressed on the same polycistronic mRNA with aadA spectinomycin resistance gene. Workability of the chloroplast transformation vectors pCA3-LUX-TrnI/TrnA and pCA3-LUX-rps12/TrnV has been similarly confirmed in E. coli prior to their use in generation ofautoluminescent transplastomic plants.

### Example 3

### Generation of Transplastomic Plants

Transplastomic Nicotiana tabacum (tobacco) plants have been generated according to methods extensively described in literature (highly detailed protocol can be found in Lutz K. A., Svab Z., Maliga P. (2006) “Construction of marker-free transplastomic tobacco using the Cre-loxP site-specific recombination system.” Nat Protoc. 1(2):900-10). Briefly, 0.6 micron gold particles (BioRad) coated with either pCA3-LUX-TrnI/TrnA or pCA3-LUX-rps12/TrnV vector DNA were bombarded into leaves of aseptically grown 4-6 weeks old tobacco plants (cv. Petit Havana) using PDS-1000/He Biolistic Particle Delivery System (system settings: bombardment He pressure approx. 250 psi above rapture disk pressure, [rapture disks of 1,100 psi were used]; distance from the top of the chamber 9 cm [third slot], chamber vacuum pressure 28 in Hg). The bombarded leaves have been incubated at 25-26° C. in dark for 2-3 days and dissected to 5×5 mm squares, which were placed in deep Petri dishes containing 50 ml of RMOP medium (RMOP per liter: MS salts, Caisson, cat#MSP01, according to manufacturer's instructions; 100 mg myo-inositol; 1 mg thiamine HCl; 1 mg 6-benzylamino purine; 0.1 mg 1-naphthaleneacetic acid; 30 gr sucrose; 6 g phytoblend, (Caisson), pH=5.8 adjusted with KOH), supplemented with 500 μg/ml of spectinomycin (Sigma). The Petri dishes were sealed with parafilm and cultivated under cool-white fluorescent lamps (1,900-2,000 lux) with 16 h light/8 h dark cycle at 26° C. Transgenic plants, shown in FIG. 8B, appeared within 4-8 weeks past bombardment. The plants have been transferred and further aseptically maintained in magenta boxes on MSO medium (MSO per liter: MS-salts, Caisson, cat# MSP01, according to manufacturer's instructions; 30 gr sucrose; 6 g phytoblend (Caisson), pH=5.8 adjusted with KOH) supplemented with 500 μg/ml of spectinomycin (Sigma) under cool-white fluorescent lamps (1,900-2,000 lux) with 16 h light/8 h dark cycle at 26° C.

### Example 4

### Identification of the Transplastomic Plants

One of the challenges in generation of tobacco transplastomic plants is appearance of plant mutants, which can be mistakenly recognized as “true” transplastomic plants. During generation of transgenic plants, genetically modified plant tissue is selected via growth medium supplemented with hormones, promoting regeneration of a full plant from a single cell, and a selective antibiotic, eradicating non-transformed plant cells. Genetically modified plant cells, giving rise to transplastomic plants, carry in their transgenic DNA an aadA gene confirming resistance to spectinomycin, the antibiotic used during selection process to kill-off non-transformed cells. However, some of the non-transformed plant cells, meant to be eradicated during the selection process, possess a naturally occurring mutation in their plastid small ribosomal RNA (rrn16) gene, which allows them to survive the spectinomycin selection. The total number of plants obtained from the bombarded plant tissue will normally contain 10-25% of wild type tobacco plants bearing spectinomycin resistant ribosomal mutation, and hence true transplastomic plants must be further identified. Several methods, such as PCR, Southern Blot or resistance to streptomycin (as the aadA gene confers resistance to both spectinomycin and streptomycin antibiotics, while ribosomal RNA mutation only tolerates spectinomycin) can be used. We have chosen to use junction PCR approach to positively identify true transplastomic plants, since it yields highly precise results in a very short time.

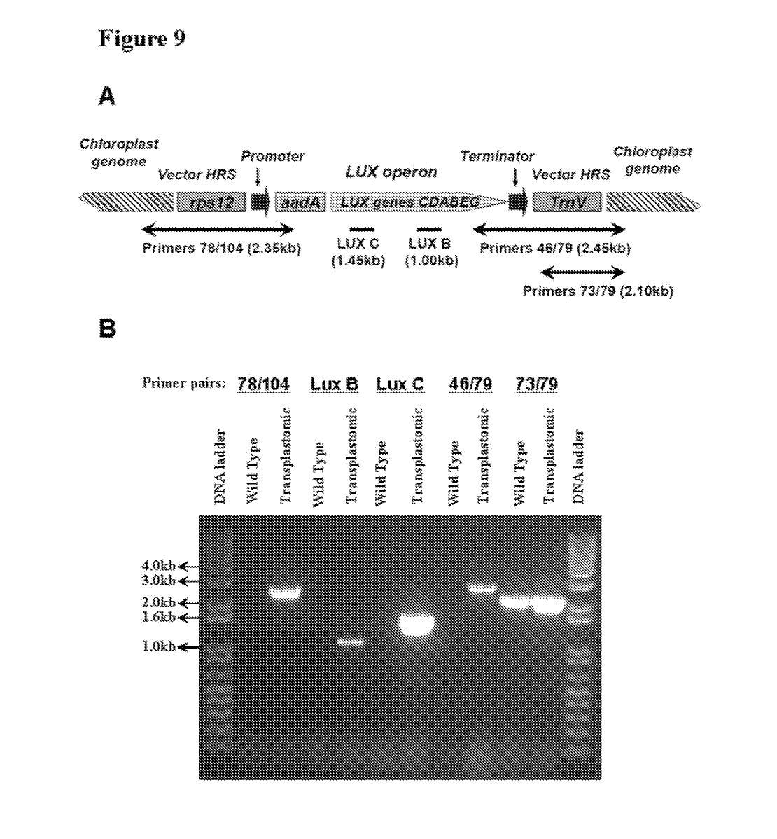

In junction PCR method, one of the primers is located within the chloroplast-integrated expression cassette and the second primer is positioned on the chloroplast genome, outside of any vector sequences (homologues recombination sequences—vector HRS—are located between the two primers), thus leading to amplification of genome-transgene junction. The junction PCR produces positive results only if the transgenes have been integrated into the chloroplast genome. Example of use of junction PCR method for identification of transplastomic plants generated using pCA3-LUX-rps12/TrnV vector is shown in FIG. 9. Panel A schematically represents DNA fragments amplified from the transplastomic plants DNA generated using pCA3-LUX-rps12/TrnV vector. Panel B demonstrates the actual PCR fragment resolved on an agarose gel (wild type tobacco DNA was used as negative control). The 2.35 kb fragment amplified using primers #78 (5′-TTGAGTATCCGTTTCCCTCC-3′) (SEQ ID NO: 90), located on the chloroplast genome outside of the vector homologues recombination sequences (HRS), and #104 (5′-CCAGCAAATCAATATCACTGTGTGG-3′) (SEQ ID NO: 91), located within aadA gene in the vector sequences (FIG. 9A), can be produced only when vector expression cassette is integrated within the rps12/TrnV chloroplast locus. Similarly, the 2.45 kb fragment amplified using primers #79 (5′-AAGCTCATGAGCTTGGTCTTAC-3′) (SEQ ID NO: 92), located on the chloroplast genome outside of the vector homologues recombination sequences (HRS), and #46 (5′-CAGATTTATCTGACTTTGATATCTATG-3′) (SEQ ID NO: 93), located within the LUXoperon in the vector sequences, can be produced only when LUX operon is integrated within this locus. As shown in FIG. 9B, the pCA3-LUX-rps12/TrnV expression cassette has undoubtedly been integrated into the chloroplast genome of the analyzed transgenic plants as all junction PCR reactions produced clear single bands of the exact expected size.

Furthermore, we have performed additional PCR reactions of the internal expression cassette genes, for LUX genes B and C (FIG. 9B), in order to further confirm presence of the LUX operon within the transplastomic genome. PCR reaction with primer pairs specific for the LuxB (5′-ATGAATTTCGGGTTATTTTTCC-3′ (SEQ ID NO: 94) and 5′-TTATTTAATAAGGTTATCTTTG-3′) (SEQ ID NO: 95) and LuxC genes (5′-ATGATAAGAAGATCCCAATGA-3′ (SEQ ID NO: 96) and 5′-CTACGGTACAAATACGAGGAAC-3′) (SEQ ID NO: 97), using transplastomic plant DNA (and wild type tobacco DNA as negative control), has further confirmed integration of the LUX operon into the tobacco chloroplast genome. Please note that primers #73 (5′-AATTGAATCCGATTTTGACCATTATTTTC-3′) (SEQ ID NO: 98) and #79 (5′-AAGCTCATGAGCTTGGTCTTAC-3′) (SEQ ID NO: 99) are designed to amplify a region of native chloroplast genome and used as positive controls for PCR reaction of both wild type and transgenic plants. Clearly positive outcome of the above described PCR analysis has revealed that we have indeed obtained transplastomic plants bearing LUX operon within their chloroplast genome. Transplastomic plants generated using pCAS3-LUX-TrnI/TrnA chloroplast transformation vector have been identified in a similar manner using junction PCR primers specific for the TrnI (5′-CGTTCGCAAGAATGAAACTCAAAGG-3′) (SEQ ID NO: 100) and TrnA (5′-CGCTGATTCTTCAACATCAGTCG-3′) (SEQ ID NO: 101) loci.

Importantly, each plant cell contains multiple copies of plastid genomes, up to 10,000 copies per cell. During the transformation event, only a few copies of plastidal genomes are transformed, and the first generation of transplastomic plants is therefore chimeric, containing a mixture of wild-type and transgenic genomes. To reach homoplastomy, where all copies of plastidal DNA in the plant contain the transgene, a second (and sometimes third) round of selection on spectinmycin is required. For the second round of selection, leaves of the initially obtained trasplastomic plants are cut into 5×5 mm pieces and placed on RMOP medium containing 500 μg/ml spectinomycin. New, second round plants, regenerating from the leaves cutting within 3-4 weeks are transferred into magenta boxes containing MSO medium for rooting. Plants with developed roots are cleaned from the MSO medium and transferred to soil in a greenhouse. Magenta-boxes grown plants must be acclimatized to lower humidity conditions during transfer to soil. For this, the pots containing the transferred plants are covered with seran wrap for the first 24 hours, which is then gradually removed within the next 1-2 days. Finally, the homoplastomy of the transgenic plants is confirmed using Southern Blot as known in the art (for example protocol see Lutz K. A., Svab Z., Maliga P. (2006) “Construction of marker-free transplastomic tobacco using the Cre-loxP site-specific recombination system.” Nat Protoc. 1(2):900-10).

### Example 5

### Characterization of the Autonomously Luminescent Plants

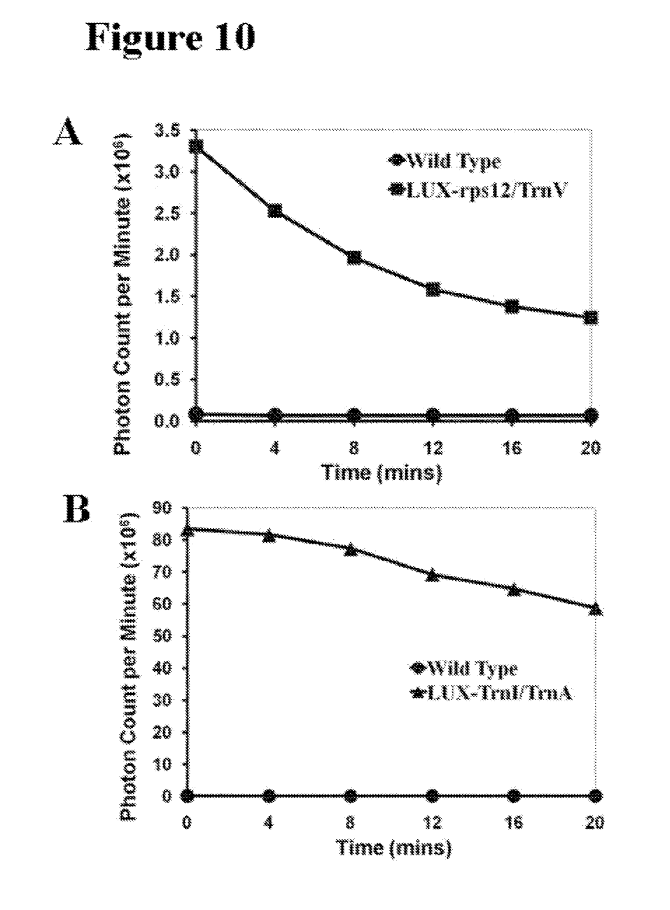

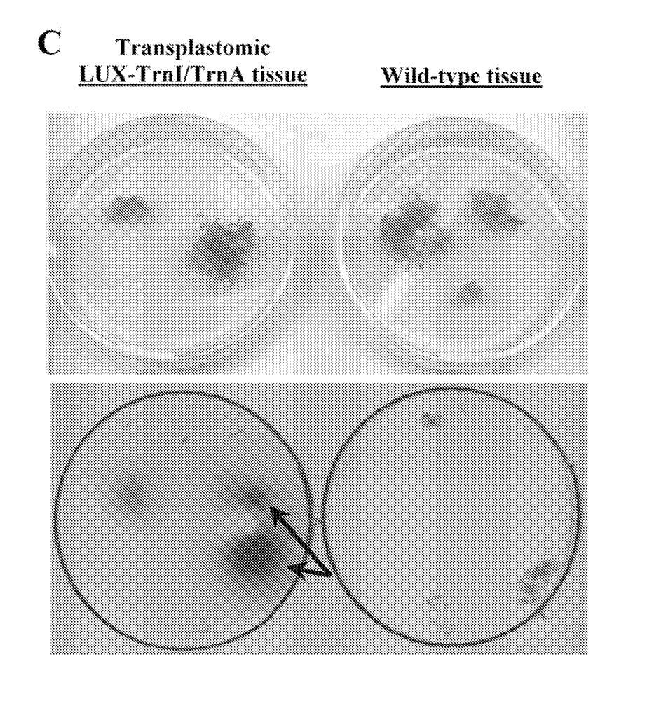

After identification of transplatomic tobacco plants containing LUX operon integrated within either TrnI/TrnA or rps12/TrnV locus, as described in examples 1-4, light emission properties of these transgenic organisms have been characterized. First, tissue from the initial transplastomic shoots, appearing after the bombardment (Example 3), has been tested for light emission using scintillation counter (LS 6500 Multi-purpose scintillation counter, Beckman Coulter). Newly appearing transplatomic shoots and wild type tobacco tissue (to be used as negative control) normalized to approx. 150 mg each have been placed in scintillation counter vials, incubated in dark for 5-10 mins to eliminate autofluoresence, and photon count has been recorded for 20 minutes (FIGS. 10 A and B). Tissue samples from transplatomic plants having LUX operon integrated in rps12/TrnV locus were designated as LUX-rps12/TrnV, and those obtained from transplastomic plant with LUX operon integrated into TrnI/TrnA locus were correspondingly designated as LUX-TrnI/TrnA. As can be seen in FIGS. 10 A and B, the transplastomic LUX plant tissue has emitted a very significant number of photons of visible light, with LUX-rps12/TrnV and LUX-TrnI/TrnA initially emitting around 3.3×10⁶ and 82.0×10⁶ photons/min, respectively, while baseline noise for the wild type non-emitting tissue was recorded at only 60-70×10³. We have also noted a decline in the luminescence levels during the experiment (FIGS. 10 A and B), apparently resulting from depletion of oxygen level from the tightly closed scintillation vials. Furthermore, LUX-TrnI/TrnA plants emitted roughly 25 times more photons from the same amount of tissue then LUX-rps12/TrnV plants. This is likely to result from much higher read-through transcriptional activity at the TrnI/TrnA locus, compared to the rps12/TrnV locus, consecutively resulting in higher expression of the LUX proteins in the LUX-TrnI/TrnA plants, and thus significantly higher light emission. These findings demonstrate that regulation of LUX transcriptional activity by the use of various promoters, as well as other genetic, transcriptional and translational elements and methods as described in above, is likely to be instrumental in modulating light emission levels from the transplastomic LUX plants.

Shortly past the scintillation counter experiments, when we have managed to grow relatively large pieces of the transplatomic LUX tissue, we've exposed it to a photographic film. As shown in FIG. 10 C, overnight exposure of LUX-TrnI/TrnA transplastomic tissue has resulted in a defined and focused detection of light emission around the transplastomic tissue, while no light emission has been detected with the wild-type tissue. Please note that exposure foci coincide precisely with the position of the transplastomic tissue on the plate. With this, for the larger transplastomic tissue section (right lower side of the transplastomic tissue plate), light emission was not homogeneous across the whole specimen and has been concentrated in an 8-shaped two distinct foci (marked with an arrows). This is likely to result from the fact that developing transplastomic plants, obtained after initial bombardment, are chimeric and contain sectors of both wild type and transplastomic tissue. The highly-emitting foci are expected to contain larger number of transformed plastidal copies then lower emitting foci.

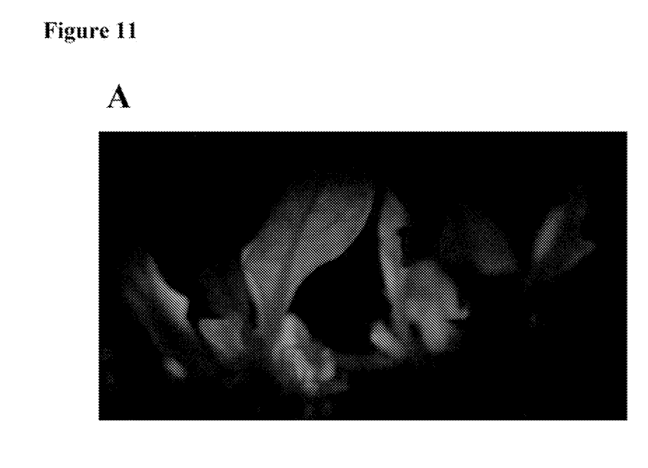

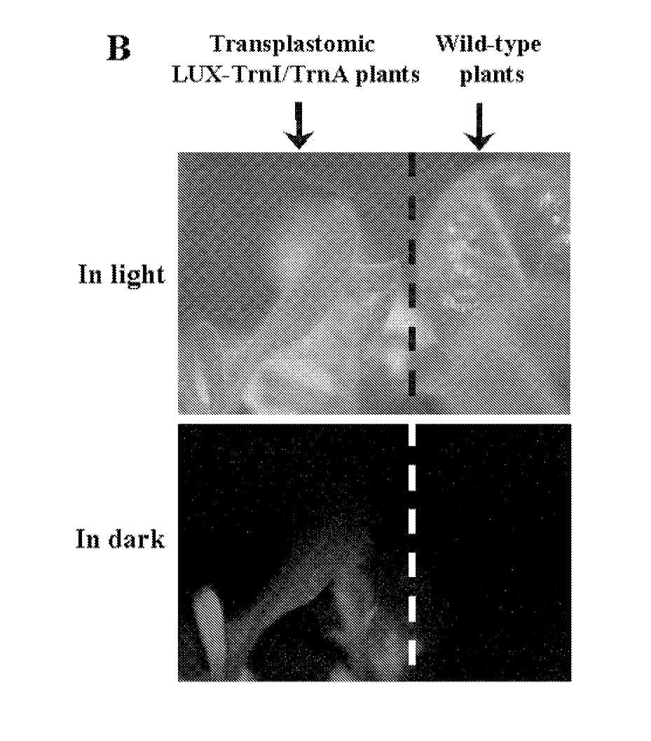

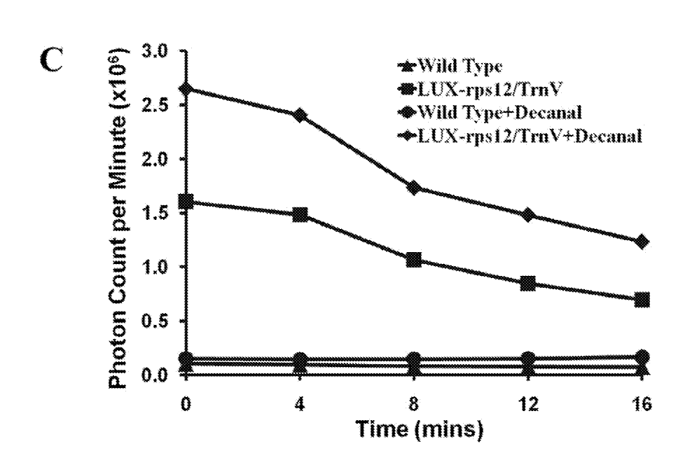

Finally, when we have obtained the fully grown transplastomic plants, we were able to photograph, as shown in FIGS. 11 A and B, using hand-held consumer camera [Nikon D200; AF-S Micro Nikkor 105.0 mm 1:2.8 G ED lens; exposures 5 min at f/4.5, 105 mm focal length, ISO 3200]. Significantly, the glow of the LUX transplastomic plants is clearly visible by a naked eye in a dark room, after about 5-10 mins eye adjustment to darkness. They just glow.

### Example 6

### Modifying Plant Autoluminescence

While we were able to generate the first ever autonomously glowing plants, clearly visible to a naked human eye, the glow effect might need further improvements in the future in regards to glow intensity, color, etc. There are multiple methods to do so were outlined above. We have performed a simulation experiment to demonstrate feasibility of these approaches. We have simulated increase in the aldehyde substrate levels, for example achievable through genetic engineering of plant phospholipid synthesis pathways, by exogenous addition of decanal. Decanal is a known substrate of the bacterial luciferase, and its exogeneous addition simulates increased and/or modified production of phospholipids in an accordingly genetically engineered plant. Small sections of LUX-rps12/TrnV and wild-type plant tissue have been placed in the scintillation counter vials, submerged in water, and autoluminescence levels were measured. Then, the vials have been opened to allow oxygen access, the samples have been supplemented with decanal to final concentration of 2 mM and autoluminescence levels have been recorded again. As demonstrated in FIG. 11C, addition of decanal has increased the autoluminescence approximately two times, confirming that increase in luciferin concentration does indeed increase light emission levels. In conclusion, similar increase in luciferase substrates levels, achieved by methods of plant genetic engineering outlined above, will increase plant light emission effect to a desired level. Other described methods can, correspondingly, be used to modify the glow in respect to color, tissue specificity and other parameters.

1-23. (canceled)

24. A transgenic autoluminescent plant, comprising:
a heterologous nucleotide sequence integrated into a plastid genome, comprising a bacterial LUX operon comprising LUX A, LUX B, LUX C, LUX D, LUX E, and LUX G genes operably linked for expression to a modified Prrn promoter, wherein said modified Prrn promoter is selected from the group consisting of a nucleotide sequence comprising:
a) a nucleotide sequence that is at least 90% identical to positions 1 to 39, 46 to 63, and 70-95 of the nucleotide sequence shown in SEQ ID NO:32, and
wherein said modified Prrn promoter is 100% identical to positions 40-45 and positions 64-69 of the sequence shown in SEQ ID NO:32; and
b) a nucleotide sequence that is at least 95% identical to positions 1 to 39, 46 to 63, and 70-95 of the nucleotide sequence shown in SEQ ID NO:32, and
wherein said modified Prrn promoter is 100% identical to positions 64-69 of the sequence shown in SEQ ID NO:32.

25. A transgenic autoluminescent plant, comprising a heterologous nucleotide sequence integrated into a plastid genome, comprising a bacterial LUX operon comprising LUX A, LUX B, LUX C, LUX D, LUX E, and LUX G genes operably linked for expression to a Prrn promoter comprising a nucleotide sequence selected from the group consisting of SEQ ID NO:30 and SEQ ID NO:31.

26. The transgenic autoluminescent plant of claim 24 or 25, wherein said heterologous nucleotide sequence further comprises a translational leader sequence functional in a plastid, operably linked to said heterologous nucleotide sequence.

27. The transgenic autoluminescent plant of claim 26, wherein said translational leader sequence is of chloroplast origin.

28. The transgenic autoluminescent plant of claim 26, wherein said heterologous nucleotide sequence further comprises at least one nucleotide sequence encoding a cofactor.

29. The transgenic autoluminescent plant of claim 28, wherein said cofactor comprises a member selected from the group consisting of:
a polypeptide encoded by a LUX H gene and/or a riboflavin (RIB) operon,
a bacterial or plant acyl carrier protein, and
a flavin reductase enzyme.

30. The transgenic autoluminescent plant of claim 29, wherein said flavin reductase enzyme is E. coli FRE.

31. The transgenic autoluminescent plant of claim 28, further comprising a second heterologous nucleotide sequence encoding a fluorescent protein.

32. The transgenic autoluminescent plant of claim 24 or 25, wherein said heterologous nucleotide sequence further comprises a sterility operon.

33. The transgenic autoluminescent plant of claim 24 or 25, wherein said bacterial LUX operon is obtainable from a bacterium from a genus selected from the group consisting of the genera Vibrio, Photobacterium, and Xenorhabdus.

34. The transgenic autoluminescent plant of claim 24 or 25, wherein said plastid is a chloroplast.

35. A vector system, comprising:
a) a plastid transformation vector, containing a first heterologous nucleotide sequence comprising a bacterial LUX operon comprising LUX A, LUX B, LUX C, LUX D, LUX E, and LUX G genes operably linked for expression to a first promoter inducible by a phage polymerase,
wherein said first heterologous nucleotide sequence is integratable into a plastid genome, and
b) a plant nucleus transformation vector comprising a second heterologous nucleotide sequence encoding a phage polymerase, operably linked for expression to a second promoter.

36. A transgenic autoluminescent plant, comprising:
a) a first heterologous nucleotide sequence comprising LUX A, LUX B, LUX C, LUX D, LUX E, and LUX G genes operably linked for expression to a first promoter inducible by a phage polymerase, integrated into a plastid genome, and
b) a second heterologous nucleotide sequence encoding said phage polymerase operably linked for expression to a second promoter, integrated into said plant's nuclear genome.

37. The transgenic autoluminescent plant of claim 36, wherein said first promoter is the T7 promoter, and said second heterologous nucleotide sequence encodes phage T7 RNA polymerase.

38. The transgenic autoluminescent plant of claim 36, wherein said phage polymerase further comprises a plastid targeting sequence.

39. The transgenic autoluminescent plant of claim 36, wherein said second promoter is an inducible promoter, a tissue-specific promoter, a circadian rhythm promoter, or a constitutive promoter.

40. A vector system, comprising:
a) a plastid transformation vector comprising a first heterologous nucleotide sequence comprising any one or more, but not all, of LUX genes LUX A, LUX B, LUX C, LUX D, LUX E, or LUX G operably linked for expression to a first promoter, and which is integratable into a plastid genome, and
b) a plant nucleus transformation vector comprising a second heterologous nucleotide sequence comprising all or some of the remaining LUX genes not present in said plastid transformation vector of a), operably linked for expression to a second promoter.

41. A transgenic autoluminescent plant, comprising:
a) a first heterologous nucleotide sequence comprising any one or more, but not all, of LUX genes LUX A, LUX B, LUX C, LUX D, LUX E, or LUX G, wherein said first heterologous nucleotide sequence is operably linked for expression to a first promoter, and which is integrated into a plastid genome, and
b) a second heterologous nucleotide sequence comprising all or some of the remaining LUX genes not present in said plastid genome, operably linked for expression to a second promoter, and which is integrated into said plant's nuclear genome.

42. The transgenic autoluminescent plant of claim 41, wherein said first heterologous nucleotide sequence comprises LUX B, LUX C, LUX D, LUX E, and LUX G genes or, alternatively, LUX B, LUX C, LUX D and LUX E genes, and said second heterologous nucleotide sequence comprises a LUX A gene.

43. The transgenic autoluminescent plant of claim 41, wherein said second heterologous nucleotide sequence further comprises a plastid targeting sequence operably linked to said second heterologous nucleotide sequence.

44. The transgenic autoluminescent plant of claim 41, wherein said second promoter is an inducible promoter, a tissue-specific promoter, a circadian rhythm promoter, or a constitutive promoter.


SEQUENCE LISTING (101 sequences)

SEQ ID NO: 1 - DNA, 5616 bp, Photobacterium phosphoreum
gctaaactcagcagttttccctggcggccaaggtggcccattaatgcatgttatcgcagcaaaagcggttgcatttaaagaagcaatggagcctgagttcaaagtttaccaagcaaatgtggttgtaaacgcaaaagcaatggttgatgagtttatcaagcgtggttataacattgtctctggtagtactgaaaatcacttgttcctagtggacttgattgataaaggtattacaggaaaagaagcggatgcagcactaggtgcagctaatattactgttaataaaaatagcgttccaaatgacccacgtagtccatttgtaacttcaggtattcgtgttggtacgccttcaattacacgtcgtggttttaacgctgatgatgcacgtcaacttgcgggttggatgtgtgatgtgttagataacgtcaatgatattgcggttattgaagcaaccaaaatgaaagtacttgaaatttgtaagcgccttcctgtttacgcataatgtggtgatggtgcggtgatttaatccccatcattacacgtcatataaggcttgcctgtttataggtgagccttttttgttttattaataaagtaacaattataattattaatgatgattgttactttttaagccaatttattagaaaaaaaggggtgatagtcttattaaagtgatgaaatgggttacactgagtcgattgaatgtaggaggctaaatgcattgtcctttttgcggtgcaaatgacaccaaagtaatcgattcccgattggttgcagatggccaccaagtgcgccgacgccgccaatgtttagcgtgtaatgaacgctatactacgtttgaaacggcagagcttgttatgcctcgcgtagttaaaacaaatggtaaccgtgatccgtttaatgaagataaattacgcggcggtattcaacgtgcgttagaaaaacgtcctgtgagtactgatgatattgaacgtgcgatcaatagcattaaatcttgccttcgagcaacgggtgaacgtgaagtcccgtcagagatgataggtaatttagttatggaagcattaaaagaattggataaagttgcttatatccgttttgcttctgtttatcgcagttttgaagatattcgtgagtttggtgaagaaatcgccaagctggaacgttaagactaataaataatgacacacactgatcaacaaatgatgtcgcgagcgatagcgcttgccaagcatggctgttataccaccgcacctaatccaaatgttgggtgtgtgattgtcaatgatggcgacattgtcggtgaaggttatcattatcaagccgggcaacctcatgctgaagtgtttgccttacgacaggcacagcagcgtgcacaaggtgcaacggtatatgtaaccttagagccttgctctcactatggacgcacgccaccttgtgctgaagccttaatcaatgccaaggtcgcacgggttgtctgtgctatggttgatcctaaccccgccgttgctggacgaggtattgccttattgcgagcagctggtattgaggttgatgtcggtgtcttagcatcgcaagcacaagcactcaatccaggctttattaagcaaatgtcacatcaaatgccgtatgtagagctcaaactagcggcgagtttagatggacgaacagcattagctaatggtgtcagtaaatggataaccggtcctaaagcgcgtgctgacgtacaacattttcgtgcgcaggctggagcgatcttatcaaccagtgcgacggttattgcagatgatccttcactgaatgtccgctggagtgaattagaagagtcagtacaacaagcttaccctcaatccgcattacgtcagcctatacgggttattattgatagtcagaatcgactcacgcctgaataccaactgtttcatcttccgggagagacgatccttgctcgaactgaaatcggtacagaagcgtggcctgattcagtacagcaatggcaaatccccacccaaactgatagtcatcagcttgatttagttgttttaatgtcgcggttggcaaattacggtattaatcatatttgggttgaagctggcgctcgtctggctggagcattattgcaacagcagctagttgatagtttgattttatatcaagcacctaagttgatgggcagtgatagccgtggtttgattgatatcactgggctgacagcaatgtcacaaacaccgctattaacgataactgatgttggtatgattgattgtgatattcgcattatagcaaccgtacacaataataaatcaagtagtgaaattaagggtttatcatgttcactggaattatagaagctgtcggtaaaatatctgcgctaacacctaaaggtgctgatgtttctgttaccgttgattcaggattattagatttagctgatgttaagttaggcgatagtattgccactaacggcgtatgtctcactgtcgtaaaactgacgggtaaaggttatgttgctgatctttcattagaaacattaaaccgtactgcatttgcaaattataaagccggccaagtcgttaatttagaaaaagccatgttagcaaccacgcgttttggtgggcatatggtttctggtcatattgatgctgtagctgaagtgattgagcggcagcatattggtcgtgcaattgaattttggattaaagtaccgccacagcttgcaaaatatatttcagagaaaggttcggtggctgtcgatggtatcagcttgacggtgaatgctgttcgtggcaatgagtttaaattaacgattgtgccacatactgctgctgaaacgacaatggctgatttcaatgttggtcgtaaaattaaccttgaggttgatgttatcgcgcgttaccttgaacggttaatgcttggtgaaaaagcagcagagaaacaatctaaagtaacgatggatttattggcgagaacagggtttctcggttaagcattgctttatggtcaataaagcaacataagaataagggtagtattatggctttaagtagtgcaaaagaaatcattgatgatattcgccaaggaaaaatggttattttgatggatgatgaagatcgcgaaaatgaaggcgatctgattattgcatcagaaaaaatcactcctgaaaccattaactttatggcaatgtatggtcgtggtttaatttgcttaacattaagtaaagcgcgttgtcagaaattgggcttaccattgatggttcaagataatactgaacaatttggtacaccttttaccatttctattgaagctgcaacaggcgtaacaaccggtatttccgccgcagatcgtgcccgcactgtacaagcagccgttgctgctgatgcgacagcggctaatattgtaatgcctggtcatatctttccattgatggcacaagaaggcggcgtacttactcgcgcaggccatactgaagcgggttgtgatattgcgcgtttagcaggtcttgaaccatcaagcgttattgtagagatcttgaatgaagatggcaccatggcgcgtcgtccacaattagagatttttgctgagaaacatggtctaaagctcggtacaattgctgatcttattgaataccgtaatcaccatgaaaccaccattgaacgtgttggtgaatgtaagctcaatactgaatttggtgagtttgatatgatcacttatcgcgataagattgatgatcaaattcattatgcgttgtgtaaaggtgacattgaagctgatgctgcgacattagtacgtgtgcacttacaagacacctttaaagatattctacaatcaggtgcgactcagtggacattgcctgcagcaatgcaacgtatcagtgcagaaaatggggtactggtgatactgagtaagcaagaatcaactgatagtattattaacaaagttaaaaatattgcggctgagaaagaaggccgaccacaagtgaagatgagtccatataatccatcacgccaagttggtgttggttcgcaaattttatctgatcttggtattggtaagatgcgtctattatcatcaagcacacaacgttatcattcactgtcaggttttggccttgaagtcgttgaatatatttgcgaataacgatctattttgactaacagcatttaggtgttgagcattattatgattgataatgtgtgcttattggttggttctacaacgaataagcaataaattttaacaaatcctctgaaaccgacggttgtgcgtagcagacatatcgtaaagtgtgttagactcccgcagtttctcaaaccggaaagacatagtcacaggaaggcccatgaaggtaattgaaggcgccatcgcggcaccaaacgcaaaaattgctatcgtaattgcacgctttaatagctttattaacgaaagtttactttcaggtgcattagatgcgctgaaacgtcaaggtcaggttagtgaagataatatcactgttgttcgttgccctggtgcttatgaactaccgcttgttgcccagcaagtcgcgaagagtgatcgctatgatgctatcgtagcattaggctcagttattcgtggtggaacacctcattttgactatgttgccggtgagtgtaataaaggtctagcacaagttgcactagagtataatactccagttgctttcggtgtgttgactgttgattctatagaacaagccattgagcgtgccggtaccaaggctggtaataaaggggcagaggctgcactaagcgcgctcgaaatggtaaatgtcctgtcccaaatcgaatcctaatgggggttactgtgaaaccagccgcgcgtcgtaatgcacgtcaatttgctgtacaagcaatttattcttggcaacttactaaaggtaatgttgctgatatcgaacaatatttcctttctggtgataagtttgaagaagaagaacatcaagctgatgcgcctgtatgtaaagcgcctgatactgatgttagctacttccgtgatttatttgcaggtgttgtattaagccatcaagaacttgatagcaaaatgcgcccatatttgtctcgtccgcttcaggatctagaccagatggaacttgcactacttcgtcttgcaatgtacgagatggttaagcgtgaagacgtaccatttaaagttgtgattaacgaagcaatcgaattagcgaaactcttcggtgctgaagatagccacaaattcgtcaatggtgtattagataaagctgcaccatctctacgtaaaaaagataaataattttttattgcgttgagtaagatgaaagaagtcagcttattagctggcttttttttatttagggaaaagagaaccatggcaaaaaatgaatttgatattatcaaacattatttcgcacagcagcaattacaacgcgatgatgtcgctttagcaataggtgatgattgcgctttagttgatgtacctgctgatcattatgtagcggtaagtactgactctttagttgctggtacccactttttagccgatgctgatccagcattggttgcttataaagcattagcatctaatttaagtgatttagctgcaatgggtgcaacgccagcatgggtg

SEQ ID NO: 2 - DNA, 3191 bp, Vibrio fischeri
aagataagtttttagtttttgtcccatagttaaaaggaaattatatgaaagatgaaagtgctttttttacgattgatcacattatcaagcttgataatggtcagtctatccgagtttgggaaacactccctaaaaaaaacgtaccagagaaaaaacatacaatacttattgcttcgggttttgctagaagaatggatcattttgcaggtcttgctgagtatttatctactaacggttttcatgtcattcgctacgattctttgcatcatgttggattaagcagtggatgtataaatgaatttacgatgtcgattggaaaaaatagcctgcttacagtcgtagattggcttaaagagcatggtgtcgaacgaatagggctgattgctgctagtttgtcagcgagaatcgcttatgaggtagtaaataaaattaaattatcatttttaattacggccgtaggtgtcgttaatcttagagatacattagaaaaagcattggagtatgactatttgcaattacctatttcagatctaccagaagatcttgactttgaaggtcataatttaggagctgaggtctttgttacagattgctttaaacataaatgggacacattagactcgacacttagtggtgttaaaggattaacgattccatttattgcttttactgcaaacgatgatagctgggtaaagcaaagtgaagttatagagctcattgatagtattgaatctaataattgtaagctctattcgctaattggaagttcacatgatcttggggaaaatttggttgtattaagaaatttttatcaatcagtaacgaaggcagctttagcattagatgttggtttattggatttagatatagatattattgaacctcgatttgaggacgttacaagtattactgttaaggagcgtagattaaaaaatgaaattgaaaatgaattattagaattagcttaattaaataaaatcaccaaaaaggaatagagtatgaagtttggaaatatttgtttttcgtatcaaccaccaggtgaaactcataagcaagtaatggatcgctttgttcgacttggtatcgcctcagaggaagtaggctttgatacatattggaccttagaacatcattttacagagtttggtctcacgggaaatttatttgttgctgcggcaaatctgttaggaagaactaaaacattaaatgttggtactatgggggttgttattccaacagctcatcctgttcgacaattagaagacgttttattattagatcaaatgtcgaaagggcgttttaattttggaaccgttcgagggctataccataaagattttcgagtatttggtgttgatatggaagagtctcgagcgattactcaaaatttctaccagatgataatggaaagcttacaaacaggaacagttagttctgatagtgattatatccagttccctaatgtagatgtgtatcctaaagtatattctaaaaatgttccaacgtgtatgactgctgagtccgcaagtacgacagaatggctagcaatacaagggctaccaatggttcttagttggattattggtactaatgaaaaaaaagcacagatggaactctataatgaaattgcgacagaatatggccatgacatatctaaaatagatcattgtatgacttatatttgctctgttgatgatgatgcacaaaaggcgcaagatgtttgtcgggagtttctgaaaaattggtatgattcatatgtaaatgcgaccaatatctttaatgatagcaatcaaactcgtggttatgattatcataaaggccaatggcgtgattttgttttacaaggacatacaaacactaatcgacgtgttgattatagcaatggtattaaccctgtaggcactcctgagcagtgtattgaaataattcaacgtgatattgatgcgacgggtattacaaacattacatgcggatttgaagctaatggaactgaagatgaaataatagcttccatgcgacgctttatgacacaagtcgctcctttcttaaaagaacctaaataaattacttatttgatactagagataataaggaacaagttatgaaatttggattattttttctaaactttcagaaagatggaataacatctgaagaaacgttggataatatggtaaagactgtcacgttaattgattcaactaaatatcattttaatactgcctttgttaatgagcatcatttttcaaaaaatggtattgttggagcacctattaccgcagctggttttttattagggttaacaaataaattacatattggttcattaaatcaagtaattaccacccatcaccctgtacgtgtagcagaagaagccagtttattagatcaaatgtcagaggggcgctttattctcggttttagtgactgcgaaagtgatttcgaaatggagttttttaaacgtcacattccatcaaggcaacaacaatttgaagcatgctatgaaataattaatgacgcattaactacaggttattgtcatccccaaaatgatttttatgattttccaaaggtttcaattaatccacactgttacagtgataatgggcctaagcaatatgtatccgcaacatcaaaagaagtcgtcatgtgggcagcgaaaaaggcactgcctttaacatttaagtgggaggataatttagaaaccaaagagcgttatgcaattctatataataaaacagcacaacaatatggtgttgatatttcggatgttgatcatcaattaactgtaattgcgaacttaaattctgatagaagtacggctcaagaagaagtgagagaatacttaaaagactatatcactgaaacttaccctcaaatggacagggatgaaaaaattaactgtattattgaagagaatgcagtagggtctcatgatgactattatgaatcgataaaattagcggtggaaaaaacagggtctaaaaatattttattatcctttgagtcaatggctgattttaagggggtaaaagaaattattgatatgttgaaccaaaaaattgaaaagaatctaccctaataaaattaagggcaatttatatattagattgccttttttgcatttctgttgatattaggtgttattggagaggggatggtatgactgttcatactgaatataaaagaaa

SEQ ID NO: 3 - DNA, 9585 bp, Vibrio harveyi
gtgagcagcccctattattatggggtgaaagctgttgttttgtaaaaatattgggcgttctcatggaagctgagtctcacgattgcattctctttccgtttagcttagtcctagaggaaatacatccgatgaatggtccacgacatttgtaagccaacaacgttgcggcagcataaacgctctgcatagactggccgcgaggctagtttatgaatgtccgactgcttaaacgcttgcaaagtaaagagcatattggagagttacctatgacagtacatgaactagagtgccaccgcgcatcagaaacaaaagacaatgctatcgtttcagccaacgagaactttagcgccgtcagagcgctaagcgcatcatgaaggcgagagagactgctgcgcagatcacgttgaccatgaacgacttagtcgcgagcaagaacgacacaaaatgttatatggacttctctgacaagaacacaggagacaaagtatttcgtgatgacggtgaaaagatcgttatgcgcagcaaacaagcggacattaaccataccgcagccgcactaatttggagtggtgaaaattaatggtccgaaggagttgaaagagcaagtcattgatgtggctgtgagcaagggcttaaacctgcaagtgacaataaacgggaagctgccaaaccgttttgaagaatccacggtcgagcaaattgtgaagaaagatgctttcttgaagcactacaccgttaaagatgtgcagtttggtttgatagaccaaaagaaagcgtagggtgttcaaccagtgcctaagacgtatgatgctcaagctaacattatcgatgaaattggtaagcaaaaacaaagccgccatataaaatgaacactcggtttaatacgagcacaaattgtcgctgttcaatttcaaggtttagtcagatcttataggttggtctgactcattctgacctaaattagttagatcaaatcgcttaacgtattcaaacgccctagtttcttgatcccaaaactgcgtaattattcaagttttatttttaagttaccttaaaagctcacctttatgattagtgttaagacatcattaaaaaatctatgcataaattataaatactatttcttaactattgggggttattgatcggttaggtagtataagtatctaaattaatgagctttgtttagtgtttttctatttataaataactttaattataattaaagttgatggtaatgattatcattagcgtgatccttttggcgtgtttgattaaattaaattaattttcttttaaattgtaaatttaaacattaccgtgtttttaaattcaaaagagaagctcttgatatggaaaaacacttacctttaataataaatgggcaaatagtttctactaaagaaaatcgatttgagatcagttttgaagaaaaaaaagttaagattgattcctttaatcattttcatttaatccagatggtgactcatgattatttaaatgatctaaatattaataacatcatcaattttctttatacaacggggcagcgttggaagagcgaagaatattcaagaagaagggcatatattcggtctcttattacttatttggggtattcaccacaaatggcgaaactagaagcaaattggattgcaatgatcctttgctctaagagtgcgctctacgacattattgataccgagcttggctcaacgcatatacaagatgaatggctaccgcagggtgagtgttatgtgagggcttttcctaaaggacgcacgatgcatttgcttgcggggaatgttcctctctccggtgtgacctcaatactacgaggcatactgacgagaaatcaatgtattgtgagaatgtcagcatcggatccttttactgcccacgcgctagcgatgagctttattgacgtcgatccgaatcatccaatttctcgttctatctccgtattgtattggcctcatgcatcggatacgacactcgctgaagagttactcagtcatatggatgcagtggttgcttgggggggggcgggatgccattgattgggcggttaagcattctccttcacatatcgatgttttgaagtttggtccaaagaagagttttaccgtgttagaccatccagccgatctagaagaagccgcctcgggtgttgcccatgatatttgcttttatgaccaaaatgcctgcttttctactcagaatatttacttttctggagataagtatgaagaatttaaattaaaacttgttgaaaaactgaatctctatcaagaagttttaccaaaatcaaaacaaagttttgatgacgaagctttattttctatgactcgtcttgagtgtcaattttctgggttgaaagttatatcagaaccggaaaataactggatgatcatcgagtcagagcccggggttgaatataaccatccattaagtcgttgcgtttatgtccacaaaataaataaggttgatgatgttgttgaatatatagaaaaacatcaaacacaaacgatttctttttatccatgggaatcttccaagaaatatcgagatgcattcgccgcaaaaggggtagaaagaatcgttgaatctgggatgaataatatatttagagctggtggcgcacatgatgcaatgcgcccacttcaacgtttagttcgatttgtttctcatgaaagaccatataacttcaccactaaggatgtatctgtcgaaatagagcaaacccgctttcttgaagaagataaattcttggttttcgtcccttaaataaggaaaattacaatgaataatcaatgcaagactattgcacacgtgttacgcgtgaacaatggtcaggaacttcacgtctgggaaacgcccccaaaagaaaatgtgccttctaaaaataacaccattttaattgcttctggttttgccagaagaatggatcactttgctgggttggccgagtatctatctgaaaatggtttccatgtgttccgttatgactctcttcatcatgtggggctcagctcgggttcgattgatgaattcaccatgacgacgggcaaaaatagtttgtgcactgtttatcactggctgcagaccaagggcacacaaaacattggcttgattgcggcaagtctctcagctcgtgtcgcctatgaagttatctctgatctggagctgtcttttctgattactgcggttggtgtggtgaacttgcgtgacacactagaaaaagcgcttggttttgattacctcagtttgcctatcgatgagctaccaaacgatcttgattttgaaggtcataagcttggttctgaagtgttcgttcgcgactgcttcgagcatcactgggataccttagattctactctcgacaaagtagccaatacctcggttcctttaatcgcctttaccgctaacaacgatgattgggttaagcaagaagaagtctatgacatgttagcgcatatccgcactgggcattgcaagctctactccttgcttggtagctctcatgacttgggcgaaaacttggtcgtgttacgtaatttttaccaatccgtcaccaaagccgccatcgcaatggatggaggcagcttagaaatcgacgtcgactttatcgagcctgattttgaacaactcaccatcgcgactgtgaatgaacgtcgcttgaaagcggaaattgaaagccgtacgccagaaatggcttaggtctcatcgtaataccaataaataaggaaatgttatgaaatttggaaacttccttctcacttatcagccacctgagctatctcagaccgaagtgatgaagcgattggttaatctgggcaaagcgtctgaaggttgtggtttcgacactgtttggttactagagcaccacttcactgaatttgggttgttagggaatccttatgttgctgccgcacatctattaggtgcgacagaaaagctcaatgttggcaccgcagccattgtattgccgactgcccatccggttcgacaagcagaagacgtaaacctactggatcaaatgtcgaaaggacgattccgttttgggatttgtcgcggtttgtacgacaaagatttccgtgtctttggtacagacatggataacagccgagccttaatggactgttggtatgacttgatgaaagaaggcttcaatgaaggctatatcgcggcggataacgaacatattaagttcccgaaaatccaactgaatccatcggcttacacacaaggtggcgctcctgtttatgtcgtcgcggagtcagcatcaacgacagaatgggctgcagagcgtggcctaccaatgattctaagctggatcatcaatactcacgagaagaaagcgcagcttgatctttacaatgaagtcgcgactgaacatggctacgatgtgactaagattgaccactgtttgtcttacatcacctccgtcgatcatgactcaaataaagccaaagatatttgccgcaacttcttgggtcattggtacgactcatacgtgaatgccaccaagatttttgacgactctgaccaaacaaaaggctacgacttcaataaaggccaatggcgcgattttgtgttgaaaggccacaaagataccaaccgccgaattgattacagctacgaaatcaacccagtagggacgcctgaagagtgtatcgcgattatccagcaagatatcgatgcaacgggtattgacaatatttgttgtggttttgaagcaaacggttctgaagaagaaattatcgcatctatgaagctattccagtccgatgtgatgccatatctcaaagaaaaacagtaattaatattttctaaaaggaaagagacatgaaatttggattattcttcctcaattttatgaattcaaagcgttcttctgatcaagtcatcgaagaaatgttagataccgcacattacgtagatcagttgaagtttgacacgttggctgtttacgaaaaccatttctcgaacaatggtgtggttggtgccccattaacagtggctggttttttacttggtatgacaaagaacgccaaagtggcttcgttgaatcacgttattacaacacatcatccagtacgtgtggcggaagaagcgtgtctactcgaccaaatgagtgaaggccgttttgtctttggctttagtgattgtgaaaagagtgcagatatgcgcttctttaatcgaccaacggattctcagtttcagttgttcagtgagtgtcacaagatcatcaatgatgcattcactactgggtactgccatccaaacaatgatttttatagttttcctaaaatctctgttaacccacacgcttacactgaaggcggtcctgcgcaatttgtgaatgcgacgagcaaagaagtggttgaatgggcggctaagttagggcttccactcgtgtttaaatgggacgactcaaacgcgcaaagaaaagaatacgccggtttgtaccacgaagttgctcaggcacatagtgtcgatgttagtcaggttcgacacaagctgacgctgttggtcaaccaaaatgtagatggtgaagcagcaagggcagaagcacgcctgtatttggaagagtttgtccgtgaatcttacccaaataccgaatttgagcaaaaaatggcagagctgttgtcagaaaatgccatcggtacttatgaagaaagtactcaggcagcgcgagttgcgattgagtgttgtggtgccgcggacctattgatgtcttttgagtcgatggaagataaagcgcagcaaagagcggttatcgatgtggtaaacgccaacatcgtcaaataccactcgtaacgtttaactgatgctgaaggggcagcgatgccccttatatcaccattcttttcgcgcgatagcgctaactaatagaggcatttatatggacgtactttcagcggttaagcaggaaaatatcgcagcgagcacagaaatcgatgacttgattttcatgggaactcctcagcaatggtcattgcaggaacaaaaacagctgacatctcgccttgttaaaggggcatatcaataccattaccacaataatgatgactatcgtcagttctgccaaaggctgggagtcggagaggaggtcgaagatctcaatgagatccccgttttccctacttctatttttaagttgaagaccctattaacacttgacgatgaagaggtagagaatcgctttactagcagcggtactagtggcatcaaaagtattgtcgcacgagatagactcagtattgagcgacttcttggctcagtaaatttcggtatgaattacgttggtgattggtttgaccatcagatggagttggtgaacttaggcccagatcgctttaatgccaacaatatttggttcaagtacgtcatgagcttagtcgagctcctttatccgaccgcatttactgccactgaggatgagatcgactttgaggcgacgctagctaatatgaatcgtattaagcagtctggtaaaaccatttgtcttatcggccctccttattttatctatctactgtgctgtttcatgcgcgagcaaggtcaaactttcaatggtggtcgcgatctttacatcatcactggcggcggctggaaaaaacatcaggatcaatcgctcgatagagacgagttcaaccagcttttgtgtgagacttttaccttagaaagcgcagagcagattcgagacacatttaatccagttgaactgaacacctgcttttttgaagatacagaacacaaaaagcgtgtaccgccctgggtctttgcaagagctctggatcctaaaacattaaagccgcttccgcatggccagccaggactgatgagctatatggatgcctcggcggtcagctatccatgttttctagtgacggatgacatcggcatcgtgcgagaagaagaaggcgatcgcccgggaaccactgttgagatcgttagaagagttaagacgcggggtatgaaggggtgtgctctcagtatgtctcaagcatttacagctaagaatgaaggaggcaactgacatgttatgttcgatagaaaaaattgagccgttaactaacctcatattccgagtattgctcaagccagatcagccttttgaatttagggcagggcagtacattaacgtcagtttaagctttggtagtttaccgttttctatagcctcatgtccttctaatggtgcgtttttagaactccatattggtggctcagatatcagcaagaaaaatacgcttgtgatggaagaactcaccaattcatggggctgcggcaacatggttgaagtcagtgaggcgcgaggtgaggcttggttgcgtgatgagagtgtcaaacccttgttattggtcgcaggcgggacgggaatgtcatacaccctaagtattttgaaaaatagcttggagcaagggtttacccagccgatttacgtctattggggcgccaaggatatggataacctgtatgtacatgacgaactggtggatattgcgcttgaaaacaaaaacgtcagttacgtgccagtcactgaaatatcaacctgtccccaatacgctaagcaaggaaaggtgttggagtgtgtgatgagtgatttccgtaacttatctgagttcgatatctacttgtgtggtccttgcaaaatggttgaagtggctcgtgattggttctgtgacaaaagaggggcagaaccagagcaactttacgcggacgcgttcgcttatttgtaatcattatcaaggagaagaaactatgagctcaacgtcactactagatgagtttggcactccagtacaaagggtagaaagagcgattgaggctctgaaaaatggccttggtgttctattaatggatgatgaggatcgcgagaacgaaggcgaccttatcttctctgcacagcatcttaccgaagcgcaaatggcactcatgattcgtgaatgcagtggtatcgtgtgtttgtgcttaacggaggaacgcgccaattggttagagcttcctcctatggtgaaagataatcgcagtaaaaaccagaccgcttttacggtttcgattgaagcgaaagaaggggtgacgacaggagtctctgcgaaagatcgcgttacaacggttaaaacggctacttattttgatgctcaaccagaagatttagcaagaccaggccatgtttttccgctggttgcgaaaacaaatggcgtgttggcccgtcgaggtcataccgaaggtacgatcgatttgatgtatctagcaaacttagtcccatcagggatcctttgcgaactgactaaccgtgatggaaccatggcgaaactaccagaaaccattgagtttgcaagacgtcatggaatgccagtgctcactattgaagatatcgtcgattatcggacggtaattgaactgagaaatgaatatgagagtggcttagtgtgtgaagtgagttggtcttagtatttaaagttcctttgtttgttcagtcgcctagctgactttccgcccttatcgggcggtttttttgttttgtagaagtcaaaaataaacccgcaaaagcgggcttatttaagtaaggtagttggagcagttgacgtattacctgaagtgttctgactcagacctgatctgacagttacccacttttcgactcggtgcctgtcaggttatatctgggctagattcttttcagcccagattgatttcccatcctctaatgtctctattggcgttcggccgcagcacatttttccttgatgagtacgatgattgttgttgtagtccatccattcgtcagatctttctgtaactcttccattgaatcatacagtttctttctgaaggccacttggtagaactcatttaatatggccttgtggaagtgttcgcagataccatttgtctgtggcgacatcgctttagttttcgtgtggtctatatcattgatggcaagatagagttggtaatcgtgctgtttaacacggccaaagtatccggagcctcggtccagtcaaaattctcagcattagcagctcatgagtgtcgaagtacggtagtcggtgattggtgtttttgtcgtgtggagcttagcgaagcgactttgttgtaggtattagcgactgtcttctgatagatgcgtccaatacctttcaaattaccaatatagaacgtatcttgagagtcgaagtaacctgagtgtgcagtttctatctcgccaaaagcctaatcatcgtccttctaacgctcaaaggcagcgacttaccttcctttagagctataccgttctcagcaatctgttttctgagacaataaggcgtttttgactttttctaagtcattattaaaccaagtctaacgcacactacttggagatgcgatagataagacttatggagatcagcaggttgaacaaataagacaaaaaatgacatcgaaaatcaatgattattcgccatcggaacaggtttcatcgaacctatcaggcagaaaggatgctttggggaaaatagttcacggagaactaccagcaataaatagtgcactagatggtacaaaataacttttcaatatcggtatttttgt

SEQ ID NO: 4 - DNA, 6782 bp, Photorhabdus luminescens
ggaatataggggtcatcttcttctaaattaaccccaatacaatcaacatcatttaatccaatttcccaagcccaccgaacgtctaacgtactcatttctatacctaaactatctttttgtggatgttcaggattaatattttttaacatcgcatcacatcctagatagaatttcttattcagacaacggacaccgattttgaactgactggccttgcttacgacacaaaattcaatttaaaccacgaagtaaaactgttttatgattctaactggtttgaaattgtgccagcttggcgtacgttgccaatctctgttacgccgtgtatggggatactgccagccagctactatgatgctgtacgacaagccgcggcacacctcaaaaagtagctaaatttattgctttaattggaggggcgggctatatacacaaagagctcaggtattttgaacttgagctcttcttctagaaatctgctggtggaaagcggcattgccattcaatgtattctctcggtattagagaaatagcataaggagttattaaatgaataatcagtttaatcagtttcatgaatatttgtatataaagaagagcttgatttctttaatttaaatttttattaataaaattttgttgatataaaaagtttcctttgctattttacagacattacaaatggaacaggaattatatttatgagcgcgaagttacattaatagttttgagcataaaattcagggcgttattatttctaacgaaacttatagattgagattattataataaaaaatcttattttctggatattctttttatttttatcgttgtaacataaaaatacaagaagcaaatattggtacaaccagtttgcaagatgattaaacaataacttaagttgaagttaccccattaaatggatggctaatatgactaaaaaaatttcattcattattaacggccaggttgaaatttttcccgaaagtgatgatttagtgcaatccattaattttggtgataatagtgtttacctgccaatattgaataattctcatgtaaaaaacattattgattataatgaaaataataaattacggttgcataatattgtcaattttctctatacggtagggcaaagatggaaaaatgaagaatattcaagacgcaggacatacattcgtgatttaaaaaaatatatgggatattcagaagcaatggccaagttagaggccaactggatatctatgattttatgttctaaaggtggcctttatgatgttgtagaaaatgaacttggttctcgccatatcatggatgaatggctacctcaggatgaaagttatattaaggcttttccgaaaggtaagtctatacatctgttggcaggtaatgttccattatctgtgatcatgtctatattacgcgcaattttaaccaagaatcagtgtattataaaaacatcgtcaaccgatccctttaccgctaatgcattagcgttaagctttatcgatgtagaccctaatcatccgataacgcgctctttgtctgttgtatattggccacaccaaggtgatacatcactcgcaaaagaaattatgcaacatatggatgttattgtcgcttggggaggggaagatgcgattaattgggctgtagaacatgcaccaccctatgctgacgtgattaaatttggctctaaaaagagtttttgcattattgataatccagttgatttaacgtcagcagctaccggtgcggctcatgatatttgtttttacgatcagcgcgcttgtttttctgcccaaaacatatattacatgggaaatcagtatgaggaatttaagttagcgttgatagaaaaacttaatctatatgcgcatatattaccaaacgccaaaaaagattttgatgaaaaggcggcctattctttagtccaaaaagagagcttatttgctggattaaaagtagaggtggatgttcatcaacgttggatgattattgagtcaaatgcgggtgtggaatttaatcaaccacttggcagatgtgtgtatcttcatcacgtcgataatattgagcaagtattgccttatgttcaaaaaaataagacacaaaccatatctatttttccttgggaatccgcatttaagtatcgagatgcgttggcattaagaggtgcggaaaggattgtagaagcaggaatgaataatatatttcgagttggtggatctcatgacggaatgaggccgttacaacgattagtgacatatatttctcatgagaggccatctcattatactgctaaggatgttgcggttgaaatagaacagactcgattcctggaagaagataagttccttgtatttgtcccgtaataggtaaaaagtatggaaaataaatccaaatataaaaccatcgaccatgttctttgtgttgaaggaaataaaaaaattcatgtttgggaaacgctgccagaagaaaccagcccaaagagaaagaatcccattattattgcgtcgggttttgcccgaaggatggatcattttgctggtttagcggaatatttatcgcggaatgggtttcatgtgattcgctatgattcacttcaccacgttgggttgagttcagggacaattgatgaatttacaatgtctataggaaaacagagcctattagccgtggttgattggttaaatacacgaaaaataaataaccgtggtattttggcttcaagcttatctgcacggatagtttatgcaagtctatctgaaattaatgtttcatttttaatcaccgcagtcggtgttgttaacttaagatatacgcttgaaagagctttaggatttgattatctcagtttacccattaatgaattgccgaataatttggattttgaaggccataaattgggtgctgaagtctttgcgagagattgccttgattttggctgggaagatttaacttctacaatcaatagcatgatgtatcttgatataccgtttattgcttttactgcaaataacgacaattgggtaaagcaagatgaagttatcacattgttatcaaatattcgtagtaatcgatgcaagatacattctttgttaggaagttcgcatgacttgtgtgttttcttagtggtcctgcgcaatttttatcaatcggttacgaaggctgctatcgcgatggataatgatcgtctggatattgatgttgatattattgaaccatcattcgaacatctaactattgcgacagtcaatgaacgtcgaatgaaaattgagattgaaaatcaagcgatttcgctgtcttaaaacctattgggatagatattaccctatagatttcaagatggatcgcgacggcaagggagcgaatcccgggagcatagcaaactatgtgaccggggtgagtgagtgcagccaacaaagaagcaacttgaaagataacgggtatagttaattctatcactcaaatataagggctctctatgaaatttggaaactttttgcttacataccaacccccccaattttctcaaacagaagtaatgaaacgtttggttaaattaggtcgtatttctgaggagtgtggttttgatactgtatggttactggagcatcatttcacggagtttggtttgcttggtaacccttatgtcgctgctgcatatttacttggtgcaaccaaaaaattgaatgtagggactgcggctattgttcttcccaccgctcatccagtgcgccaacttgaagatgtgaatttattggatcaaatgtcaaaaggacgatttcggtttggtatttgtcgggggctttacaataaagactttcgcgtatttggcacggatatgaataacagtcgcgctttaacggagtgctggtacgggttgataaaaaatggcatgacagagggatatatggaagctgataatgaacatatcaagttccataaggtaaaagtaaacccgacagcatatagtaaaggtggagcccctgtttatgtggttgctgaatcagcctcgacaactgaatgggccgctcaatttggtttaccgatgatattaagttggattataaatactaacgaaaagaaagcacagcttgagctttataacgaggtggctcaagaatatgggcacgatattcataatatcgaccattgcttatcatatataacatctgtaaattatgactcaaataaagcgaaagagatttgtcggaaatttctagggcattggtatgattcttatgtgaatgccacgaccatttttgatgattcagacaaaacaagaggttatgatttcaataaagggcagtggcgtgactttgtattaaagggacatagagatactaatcgccgcattgattacagttacgaaatcaatcccgtgggaaccccgcaggaatgcattgacataattcaaaaagacattgatgccacgggaatatcaaatatctgttgtgggtttgaagcgaatggaacagtagacgaaattattgcttccatgaagctcttccagtctgatgtcatgccgtttcttaaagaaaaacaacgttcgctattatagtagctaaggaaaaagaaatgaaatttggattgttcttccttaacttcatcaattcaacaactgttcaagaacaaagtatagttcgcatgcaggaaataacggagtatgttgataagttgaattttgaacagattttggtgtatgaaaatcatttttcaggtaatggtgttgtcggtgctcctctgactgtttctggttttttgctcggtttaacagaaaaaattaaaattggctcattgaatcacatcattacaactcatcatcctgtccgaatagcggaggaggcttgcttactggatcaattaagcgaagggagatttattttagggtttagtgattgtgaaaaaaaagatgaaatgcgtctttttaatcgccctgttgaatatcaacagcaactatttgaagagtgttatgaaatcattaacgatgctttaacaacaggctattgtaatcccgataatgatttttatagtttccctaaaatatcggtaaacccccacgcttatacccaaggcgggcctcggagatatattacagcaaccagtcatcatattgttgaatgggcggctaaaaaaggcattcctctcatctttaagtgggatgactccaatgatgttagatatgaatatgctgaaaggtataaagccgttgctgataaatatggcattgacttatcagcgatagatcatcagttaatggtattggttaactataacgaagatagtcacaaagctaaacaagagacgcgtgcatttatccgtgattatgttcttgaaatgtatcctaatgaaaatctcgaaaataaacttgaagagataatcacagaaaacgctgtcggagattatacggaatgtatagctgcggctaagctggcaattgaaaagtgcggtgcaaaaagggtattattatcctttgaaccaatgaatgacttgatgcaccaaaaaaatgtaatcaatattgttgatgataatattaaaaagtaccacatgtagtaaaagaatatggcagcaacgctgccatattctctaaattatttggaggggtaaaacaggtatgacttcatatgttgataaacaagagatcatagcaagctcagaaattgatgatttgattttttccagcgatccattagcttggtcttacgatgaacaggaaaaaatcagaaacaaatttgttcttgatgcatttcgtaatcactataaacattgtcaagaataccgtcactactgtcaggtacacaaagtagacgacaatattacggaaattgatgacatacctgtattcccaacatcagtttttaagtttactcgcttattaacttctcaggagaacgagattgaaagttggtttaccagcagcggcacgagtggtttaaaaagtcaggtggcgcgtgacagactaagtattgagagactcttaggctctgtgagttatggcatgaaatatgttggtagttggtttgatcatcaaatagagttggtcaacttagggccagatagatttaatgctcataacatttggtttaaatatgttattagtttggtagaattattatatcccacgacatttaccgtaatggaagaacgaatagattttgttaagacattgaatagccttgagcgaataaaaaatcaagggaaagatatttgtcttatcggctcaccatactttatttatttgctctgccagtatatgaaagataaaaacatctcattttatggggataaaaacctttatatcataacggggggcggctggaaaagttatgaaaaagagtccctaaaacgcgatgatttcaatcatcttttattcgacacgttcaacctcaataatattagtcaaatccgcgatatatttaatcaagttgaactcaacacttgtttctttgaggatgaaatgcaacgtaaacgtgttccgccgtgggtatatgcgcgagcacttgatcctgaaacattgaaacctgtacctgatggaatgccgggtttgatgagttatatggatgcgtcatcaacgagttatccggcatttattgttaccgatgatgtcgggataatgagcagagaatatggtcaatatcctggtgtacttgttgagattttacgtcgcgtcaatacgagggcacagaaagggtgtgctttaagcttaaaccaagcatttaatagttgatatactctatggattttaagatgcatcgcgatggcaagggagtgaattc

SEQ ID NO: 5 - DNA, 13760 bp, Shewanella hanedai
tgtacccatacctcttgctgtatcttaatgatattaagcaggaggttatgatactcgtcatattattcacatactgggtcgagtgatattaactaagtgcctgtatatgagctaagtccatgtgctcgtatttattttcgtattgcaatgataaaaagatcgttaatactaaggtcttgttgaatgtattgacgctgacagtatttttatccaagtggcttctcattatttacgtctagttgtttaacaaatttattaaataaagctgcactcttttttgggacttctaccatgggaaggtgcccaatattcttcagtaatacgacctgaacattactgagtaaaggacagattttatcgagtgcacttttatctaaaattctgtcttcctccccccatacaatcaatactggaacctgaattttatctaagtaaggaaggagatcattgttgtgaaaatcagcaaatatcttgtgatgtaattgttttttatgcgcctgaagtgctaaatgtgtttgttttaaagatttgggaataaggccagatttatagaaagtgtaattaaagtattcaattggattggtacaatcaatcaaagggttggggcggttagaggccaagtcttgaaacatatcactcttaattgggctgtcgattccagcattatcaaataacgtgaccgataaaatctcttctggaaatagagcagcataggttgctgcgatctgccctcccatagagctaccgagtatatgaaaaggagctaatttttgctcggaaacaaacaaatgcacacgcttaacctgagcttcaatgcagtagtctgcatcaagtggtgcatcactctcaccatgtccaagtaaatctaatgcaatgattcgatattttttctttaaaaataacgacatttgaagccagttatctttattggcggtaaagccatggagcataaggagtattggcttactgtttatttgatagtgttcatttgcgttatctagatatacataacgcttttcaaggagagtgatctcttttcggctgagccctgagagataacgagtggtgtgggttaagagttgatagagatacaatggagtaaaatagataagaattaagctaattagcagtactaaaatgaatgcggatataaaaaatgaaaccatgaaactctccaaatgaaaatagattttgtctaataatatacggccccaaaagaacacttgatacagtagcacgatagttattaaagtgttgcgtaaaagtgtgctgatttgcttagcctaaattaggggtaatgattattaagatgtcagcaaacataatttgaaattaaagatttaagtggcacttggttttgaacatatttaacggttggatcttcattcatttgcactaccgcgccaatcataaatagaaagccaatacccagtagtattaatcccatagcgcatttctctggtgagttgggttctttagcacctcaacggatccagatagatgccgttaagcggggcattttgtttagcaagtttcgctgcaggtatagtatcaatatctcccaataatcttttagcaaaaacaaattgcaccctatcttgtgattgcaacaaataacgcttgatatgaaatagcgttggctaatgagtaaaatcgtattatgtcattacaatcaaactcaatggcaataactgcgcaaaatcattaacaattgggtgtgtaatttttttaattattgaacactgtgatattaataaactcttaaattatttaatgaattttgtgtttatgcgtatgggggcggttataattatagtgtttttttagtgtgatctattaagtaagattatacaggattgtgatgaatagtatgaagtattacaaagggaatggattcatgacccctgttaggatagattttgagatactttgactgcgattatttgtaccaagtttagtttgtaaattggttatatgaaaagatactgttctttcagagcaatctagtattttagatatttcccaggttgtttttccctcacttatccaagctaggcattctttctctcttggtgttaactgtgtgcaagtattgccaatatttttattaattctttgatggctgtctaaaagttcaggaagaacgagtaaaacataagagtaagagtttgaatgtaaagatattaatgttttttcattatttgaattggcaaaactaattactccaaagctattattttttgtatgaacaggaaagctaaatcctgatttaagatttgaattttttgcttcttcaattacatttgttttgtttttatcccatttactttgttccaacatactccaaattattggagcgtaattagaagcgctataatcaataataggatcgtaatcaattagtttctcatcgttgtaatattttttccaacatgaagggtaattatctaaaatatataagtctgatttaaatattgatactggattcagtatagcaaatagatagtattcacagtcaaggagaagggctaattttgtgaggcaagcgttgaggtcattatcattttcagataaacgaaggttatcattgatttcatatacggcatccattttgaaaaaatccatttatttcccttactttttataatgatatagtatttggtaactatagcatgtctagtttactaatctgtatctgtatcttaattttaaaactaaagtagtaggtggtgtgaagtaagtaatcgttttataagagttgtaaccctgtaatatcgtacaggcttaaaagatatactcgcttgttatagtggttttgaaaggagtatatatgcaaattatcataaagaaatcaaactttatcactattccaaagaatgaataccttgggttgttaaagctgcgctatcaagtatttgtgctaaggttgcaatgggggctggcttctgtaaatggtctggagtcagataactatgacaatgtggatgctgcatatatatatgcttgcgatgatactgaaaaaatttatggttgttggcggttactgccaacaacaggagattatatgttaagaacagtatttttaaagttgcttggtgaccaagatatacctaatgatcctactattattgaattaagtcggtttgcagttgaaaagcagagttcaacaatgaatggagtaagcagtgagataacaatgaaactttttgaagcaatatacattcatgcagtaaaccatggaataaaagaatacgtttcggtgacatctacagcgattgagcgatttctaaaacgaattaaaatcccttgtaatcgtattggtgatcaacaagttcatttgttgggatgtactaagtcagttgtattgtcaatgcctattaatgacgattttaaacatgctgtcatgtgttaatattaggtcaaactatgacagttaacattacattaatacgtatttaaaaacaccatttaataatggaagtaaaaagtcacaacgtaaatttatcaagggctagtaatattaacaatatagaactatcaattatactgaggtatgttaatgattaagtatgttccaatgataatcggaggcgtcattcaagatgatcccgataatgaaattagagagttaacgttaaataatgaacgtaaagttcatctacctattattgatagtagtcatgtggataaaataatagaaataaaagtacagaacaatttgaatttaaatcaagtcgttaactttttgtatacggttggacaacgttggaaaagtgaagagtattcacgtcgtcgaacttatattagagatctgaaaaacttccttggatattcaaatgaaatggcgaaactagaagctaattggattgcgatgttattgtgttcaaaaagtgcattatatgacattgtaaaaaatgatttagggtcattgcatattattgatgagtggatacctcaaggagattgttacgtcaaagcattgccaaaaggtaaatcagtacatttacttgcaggtaatgtgcccttatctggagtgacatcgattcttagggctattttaactaaaaatgaatgtattattaaaacatctgcggcagatccattcacggcaaccgctttgatatctagttttatcgatgtcaatgcgagtcatattattacgcgatcaatgtcggtgatgtattgggcacatagtgaagatatttcgttaccgaaaatcatcatgagtcaggctgatgtcgtagtcgcttggggcggagatgaggccattaaatgggctgttaaacatacaccgtacaatatcgatattctaaagtttggtccaaagcagagtctgagcattgttgataatccagaagatatggtccttgcggcaactggagttgctcatgatatttgtttttatgatcaacaggcctgtttttcaactcaaaatatttattacatcggtaataaattagctttgttcattgatgagcttgagagacaattaacactttatgcaaaaattttgcctaaaggttttcaaaattttgatgaaaaagcggcttttagtcttactgaaaaagaatgcttatttgcaggttatgacattagaaggggagataatcaaagctggctaattattacatcccctttagattcatttggaaatcaacctttatcacgatctgtttatgttcatcatgtttcaacactctctgagatactaccttttattaataaaaatgttactcagactgtctcggtaactccatggggatcctcatttaagttcagagaccaactggcagaatatggtgctgagcgtattgtcgagtcaggtatgaataatgtatttcgtgttggtggtgcccatgatggtatgcgtcctctacaacatttagttaattatgtgtctcaagagagaccatccagtcatacaacaaaagatgttgctgtggaaatagaacaaacacgttatttacaagaagataaatttttagtttttgtcccataatataaaaggaaatcatatggaatgttcaaattcttgcttaactatagatcatgttattcagcttgatgatgataaagaaattagagtatgggaaacgttaccaaaggataatacagcggtaaggaataacactattcttattgcttctggttttgctcggagaatggatcattttgcaggcttagctgaatacctatccagtaatggttttcatgttatccgttatgattctcttcatcatgttggattgagcagtggtgatattaatgagtttacaatgtcgattggaaaagacagtttgcttatcgtaatagaatggctcaaaggacgtggcgttaataaattaggtttgattgcagcaagtttatccgctcgtattgcttatgaaatatcaaatgatgttgatttatcatttttagtgactgctgtgggagttgtaaatttgagggatacgttagagaggtcattaaaatatgactacttacaattagaaattgaagacttgcctgaagatttagattttgaggggcacaatttgggatctaaagtttttgtgagagattgttttaaacataactgggttacgttagattcgacaaaaaacaaaatgaaaaacctagacattccatttattgcatttacggctaatgatgatgattgggttaagaaagctgaagtattggagatgatgaatagtatcagttctacaaagtgtaaactgtattctttaatcggtagctctcatgatttaggtgaaaaccttgtcgtgttaagaaatttttatcagtcagttactaaggccgcaatatctttagataatgattctgttgatctaaatgttgaaatatttgaaccaaaatttgaagaattaacgagtgtaacagtacaagagcgaagattaaaaaataaaatcgaaagtgaaattctagagttaatgaattaatagtcatacccggttatataaaataaaggaaataattatgaagttcggaaatatttgtttttcatatcaaccgcctggtgagactcataaacaggtaatggatcgttttattcgacttggcgttgcttcggaagaacttggctttgatacatactggactctggagcaccattttactgagttcggtcttactggtaacctttttgttgctgcagcaaatctacttggccgaactaaaacactgcaagttgggacgatgggggttgtactccctacagctcatccagttcgacaactagaagatgtattgttattggatcaaatgtctaaaggtcgttttaattttggcgttgttcgaggtttataccataaagatttcagggtatttggcgtcaatatggaagactcacgcgggataactcaaagcttccataccatgatcattgatggcgtaaaaacgggacgtataagctcagatggggaacatatagagttcccagaagttgaggtatatccaacagcttattcaaaggagctcccaacgtgtatgacagcggagtcagctagcacaacggagtggttagctgagcggggattgccaatggtgcttagctggataattggaaccaacgagaaaaaagcgcaaatggaactttataatgaaattgcgatagagcatggtcatgatattactaagattgatcattgtatgacatttatatgctcagtggataatgatagtaataaggcacgtgatgtatgccgtgcttttcttgctaattggtatgactcttatgttaatgctaccaacatattcaatgatagcaaccaaactcgtggctatgactatcacaaaggtcagtggagagattttgtactaaaaggtcatacaaatagcaacagacgtgttgattacagtaatgaaattaaccctgtaggcacacctgaagaatgtatttcaattattcaacgtgatattgatgcgaccggtattactaatatcacctgtgggtttgaagcaaatggtagtgaagaggaaatagtggcttctatgggacggtttatgacacaagtggctccttttttgaaagaccctagctagtcattaatacatttaattaaatatagtaaggaaatattatgaaatttggattgtttttcctcaactttcagctagatggtatgacttcagaaaacactttagataatatggtgagcatggtgtctcttgttgatgctgatgaatatcattttgatacagtactcatatacgaacatcatttttctaaaagtggcattatagcttcacctattacagcggctggttttttacttggattgactaataggctgcatattggctctttaaatcaagttattacaactcaccatccagtacgtgttgccgaggaatcaagtttattagaccagatgtctgaaggtcgtttcattctgggattcagcaatagtgaaaacgactttgaaatggatttctttaaacgtaatttagcatctcggcaacagcaatttgaagcttgttatgacatcattaatgaggcgttgacgactggatattgccaccctcaaaatgatttttacgatttccctaaagtgtcaataaacccacattgttttagtaaaaatgggcctaagcagtatgtagtagcaacaagtaaaagtgtcgttgaatgggccgctaaaaatgcattgtctctgacgtttaaatgggatgatagtcttgcagataaagaaagttatgcaatgctttataatgaaattgcgatgcgttatggtattgacatttcaaatgtagagcaccaacttacagtcattgtcaatttgaatgctgatggtgatttagctcgcgatgaagctaaggggtacttgaaaaactatattgttgaaacatatccagacatcgatcatgtggctaaaataaattcaatcattgcagagaacgcgattggtactgatgccgagtattatgaccaaattaaactagcagttgaaaaaacaggagttaaaaaaattctgttatcatttgaatccatgaaggattcaaatgatgttaaaaatattattaatatggcaaatgacaaaatatctaaaaatattaaggcatagtgttcgttggttttaaatggtggtagaaataatttaccactatttaaaataatgttaataacccctgctatttaggttgtatatgaatactcaatgtatagttgaagagtaccaagttatagcaagctcagagatagatgatcttatttttatgagtaacccacaagagtggacatttgaagagcaaaaagaaattcaaaataaacttatccttgaagcgtttaactatcattataatagaaatgaaagttataggaaatattgtattacacaacatgttaatgagaatatcacttcaattgatgatattcctgtgtttccaacatctgtttttaaatatatgaaactacatactgctagtgaagatgatattgagaattggtatacaagtagcggtactagtggtatgaaaagtaatattgctcgtgatcgactaagtattgaaaggttacttggttcagttaattttggcatgaaatatgtcggtaattggtttgaacacgaaatggaattggttaatcttggtccagaccgttttaacactaataatatatggtttaagtatgtaatgagtttggttgaattactttacccaaccgaattcactgtcaataacgatgaaattgatttagaaaaaacaatgcatcatttatttcgtatttataacactaaaaaagatatttgcttgattggtcctccttattttatttatctattatgtcagtacattaatgccaatgatattgaatttattgcggggaatagattgcacatcatcactggtggtggttggaagttaaatcaaagtgagtccttaagtcgtgatgattttaatgctatgttaatggaagtatttcatttgaataacatcaatcaaattagagacacttttaatcaagtagagttaaatacgtgtttttttgaagatgaatatcaaaggaagcatgttcctccttgggtttatgctcgcgctcttgatcctgaaacactacagccagttattgacggtgaacaaggattattaagctatatggatgcatcatcaacaggatacccagcatttattgttacagatgatataggtattatccatcatgttaaaagccctgatccctatcctggtacaaaagttgagataatacgaagattaaatacacgcgcacagaaagggtgtgctatttcaatgattaatacaatgaaagtaaataattaatactgcgtggtggatataatatatgaaagtaaaatgcagcgtatcaaaaatagagttaataaataaaaatatatataaagtttatattaaaccttatgttcctattgatttcaaagctgggcagtatatatatataaatttaagtggtaataaaaggcaaccattttctattgctagttgtccaacagataatagtgtgattgaactgcatgttggcagttcaaatgaaaacagttctttagatgtaatggaatattttggtgatgctctaataaaaaatagtaccatcgtgatagatgctccacatggagaggcgtggttacgtgaaggcagtaataaaccaatattgttgattgccggtggtacaggtttatcctatattagcagtattcttcgaaactgtttaaaccgaggtttcactcaacctatttatgtttattggggagtgaaaaatatagattttttgtatgcagatgaagaactacagttattatgcagtcaccacagtaatttacattacattccagtggtattagaagacagtaaatatacgtggttaggaaaaaaaggcacggttattgatgctgtcatggatgattttactgttcttacgctttttgatatttatgtctgtgggcctaatttgatgactaaggcagcaaaagataaattagttgcaaaaaagagtgctaaatcagaacaaatgttttccgatgcttttgcatatatgtgatcttttaaatgtagtgtagatataataaacatcagtctaagaagcaatctatttggttcgttgtattttaagtggtgatttctgacatgacagtcatataaagtcatgtcagttctctatgaacctcacccattaaacattaaccaaatacaaactcttcttcactgtcgagccaggttgggtattttatcattatgtcgatgaaagtagagctggctaaatgtttagctaaccaggcctgaacattgggggtaggggatgtttcgaaccacattttatctacatgggcaaattggcgtacaaagggatagattgcataatcagcgatactggctgtatgacagagcagattatcatgcttggctaagaggctttccaggtgggagatgaatatcatgccctgatcgcgataatgtgctttagtgaattcagggtatctgtcggcgtacttgtatttgtctagccaaggtttaaagttatcatcattagtcttgattaatgccatggctatctcttgtaactcaggcttatcggtcagcaataaattcagaggatccttctgcattagtgcccaacacatgatatcgatactctcatcgatcacttgattatctgaagtgatgagtacgggtaccgttccttttggggatgaatataacatttgctgaggtttattttttaggattatctctcttaacatgacttgagtgccagagagggaaatgccaagtcgagcgcgcatggcataggggcagcgtctaaatgagtagagagtggctaaagacatgattttctctggtaagttgcttgaattctttattatgttgtggcttttcttatcaaaaaacaatcaagatttaacttcaaattgaacttgggagactaaagtcatcatattttcttaatacccctactttttgtctctgtttgtgtttatgttactcatgtatgattctcgccgtttttgctaactagctcactcacaattgtgagccagtctatcattgagtcaatatgcactggagattaagctccagcaagcagtagaggttagtatgtcccacgttgttgtttgtgccctttataagtttgtttcccttcctgattttgaactgatccagaaacccctattagccgagatggaaaaatcaggcataaaaggcactttgttgctggccaacgaagggattaacggtactgtcgcgggctcacaagaagcgatagataacctacttatctggctggctggacagccgggccttgataacatagtgcataagttttcgttcgatgaaaccatgcctttttatcgtaccaaggtgaaattaaaaaaagagatcgtgacgatggggatcgagggtattgatccactcaaagtcgttggcacttatgttaagcctaaagattggaatgcgcttatttcagatcctgaggtgttactggtcgacacccgcaatgaatacgaagtcaaaataggaacctttaaaaatgctgtcgatcctaagaccgacacatttcgtgaattccctgcttatgtaaaagaacacctcgatcctgcgaagcataaaaaagtggccatgttttgtacgggtgggatccgctgtgaaaaatccaccgcttacctcaaagagcaaggctttgatgaggtttatcatcttgaaggcggcgtgcttaagtatcttgaagaggttaagcaggaagagagcttgtgggaaggtgagtgttttgtgtttgataatcgcgttgcggttaaccatgacttggaaaaaggtcaatacgatcaatgcaacgcttgtcgtatgccaataacagaggctgaaaaagccagtgaagcatttgttcagggagtcagttgtcctcattgtatcgacaccatttcagataagcagcgtcagcgctttgaagaacgagagcgtcagatgcaactcgcagacaagcgtggtgaagctcatatcggtagtgatgtcggtgctgtgattcaaaaccgccgtgatcacaaagaaaaccttaagaaagcgcagcttaagcttaacagtaaaaaatagaaataaatgagttggaggctagaatggcgtctatactcatttagcaataagagaattattgatcctgtatcgggcgattaatacctccatattgttgaaaaagccgtctttgacggctttttttttgctttctgtcattgaatgatttatttctcttgttttatccactttgatgttcatcaatgtatttttagattaactctatttagcgattgatatcgaactgtttatattctgccattgatttctttttctaatggttacttgtttgcattctatttttaagaatattctaaatagtatttctgaatttacaaatttaattatgaatgtagttttgttcaaagaaacaacaaaaataattaaataggaatagataatgaaacagtgcaaagaaaaattaatagtatcaatattatttggatgttctgctggtatagccagttgtggtgtgcaagcaatagaaatacaagctgatccatggacgttaaatattaatggtaatgttaatggccacctctcttacgtgcagtgtgataacagtactaatgtcgtcgcgggtaatccattattatgtataggcgatgatgcaacctctgttgcgaatggatatttaccaacaggtatcgatttcggtatttctagaacggttaatgattaccatgttgccgtgcattttgcttatgaagggggaaccgttactaatgggccatttaatggcggaggcacaacggagtcatttagaggatatttaactgtcgcaaatgaccattacggtgaggttaaaattggacgagactacggtgtatttggaatcgatgtcatattgtccgatatgtctttattaggcgtaggtgcttcagccattataaaatctccattaaacaccacattaggaagttctgggtatggttatatatttgtcgatcgcctcgcgcaaattaactattcgtttcctaccaaaaacggtctgagtgccactgttggggtatatcaaccgctagatccatcgaccctaggggctgaaaacacctttgtaggtgactcaggctctaaaacgccaggttttcacgggaaattaaagtatgagtttgaaaaggggtttatatctagcacttggttaacccagcagattgataataatgaggtgaacgaaaccgcttttgcttgggatgtcactggtaaaatgaattttggggcgctttctcttgtggcttcttatcatgatgcgaaaggtgtcggtcactcagggttgtttttcgatggtatcgatgcccaaggtaatgcgagaaactcgaatggctactttgtgcaggctatgtatagcttcacagacactcgtgtcggcattaattatggaatatcaacattggacagaaatgccaatgatccgttagtgaaccttaagcaaagtgaaaagctaacatttggcgcctatcatagcttgtttgaaggactcaccttggtggctgaagtgtccatgtatgaatctgaaaacaataaaaatcaaagtattgataatctaggactaaacttaggcgctgtgtattttttctagcgtagaagattaaactaaatgtaatattacgattcataaccacttagttttatggatgactaagtggtttttttgaggtgtttatggattcagttagagatcaagcaatttcaaatgttcaaattgataatgacagagtgttggt

SEQ ID NO: 6 - DNA, 7385 bp, Photobacterium leiognathi
tctagactttgtatattttcaatttaataccaaacgataaatctaagaggattttagttttttaaagataaaatacaaaatacggacaaattttttttattatcatcacaaatattttaatttaaaaacataaagaacaatcttgtatttattctaaaacaccaataatattctggtgggagatagtaatatcattaattacataccatatatagacaaacactacctagcgataataaatcccatcatacccacaccaaaaaaaaataaaaaccacccaatataacaaagacataactacaaccagaaagaattaaaataaatttcaccattcatttaagttatgtttattccatcaataataaaaactataaaaataccattcaacctaatttaaaaattccggagtaaattaaaaataaagataaaaagtaaagccaataaataaaatcacaaaaaacataaaaaacgaattaataataaaaaattttgtaatatatttcacttataccatcatctatcatgaaaaatgaaaaataaaaaaatcagaaagattaaatataaaaatattaaattaaaaactgattttattaatagtgtgaccaaaatctcgaatagaaaaaagaaaaaataaattagttatcacatccatatttctttatctaatctgattcagctagctcatgcggcatagtttataccgaaactacatactcagcatgtgcgaataccaaaggagattacatgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtagaaagagatatatcatggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaaatagtacttacctaagtacagccaaaaggaagaaataatgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaagctgttctttttaaactattcaatatcaaggcataaggaataaaatatgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataatttaattacggatagatattttcgatatatctaagtcttactaccatttatataaactatttatacagataacgtttcatttgattaagtcagtaaataattgccattaattaatggcagtgcagatccttacactgccatttataaattaaataagggttaacatgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataaaagcagggcttaatcatgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaattaaaatcattatttaactctaaataaaaccgttattaatttttcgacctacttattctgggtactgataattagtacccaatagatagttctatttatagggatatt

SEQ ID NO: 7 - DNA, 10364 bp, Photobacterium phosphoreum
ataataatctccttctgtaggtaatattatttaaatgaagttattcattttttaattagtctagtggtaaaaaaataaaattcaacagtaaataatgttttttgattttaaatctgtttttaataaataaattattgatttttaagttctgtttttgtttgtttcatttaaatatttatatttaaattgatgtttttaattacagtctatgcttgataagaagatgatttataagaaaatgtatacttaatgattactgttttatatttatactgatttttatgtattattgattttttatctatttataatgtccaaatttacttgttgaccaattttcattgtaatatttattattttacttgaaatctaaacttctaatattatatgtaataaaatgctcctgttttatatttttagatgtttaaaaatagttataaattaataaataacaaagttagatgctaaaagtgtgatatttacctcataaaattaataatgcgtgaatactttacatattaaccttacatgcccaataaaaattagctaggctatgtacatgcttatgaagcaggtttgtatgcttgctgggtatgtgcagcagggtaatttaaggagattgtatgataaagaaaatcccaatgattattggtggcgcagagagggatacttcagaacatgaatatcgtgagctcacactcaatagctataaagttagtatacctatcataaatcaagatgatgttgaggcgattaaatcacaaaacgttgaaaataacttaaatatcaatcagatagtaaacttcttatacactgttggccaaaaatggaaaagtgagaattattctcgtcgactcacctatattcgagatttgataagatttctcggatattcttctgaaatggccaaactagaagccaactggatctcaatgatcttgagttcaaaaagtgccttatacgatattgttgaaacagatttaggttctcgtcatattgtagatgaatggttacctcagggggattgttatgtcaaggctatgtcaaaaggaaaatccgttcatttgttagcaggtaatgtgcctctatctggtgttacttctattattagagcaattttgactaaaaatgaatgtatcattaaaacatcatcggctgatccatttacggcaatagcattagcttcaagttttattgatacagatgagcaccatccaattagtcgttcaatgtcggtaatgtattggtctcatagcgaagatattgtaatcccacaacaaattatgaattgtgctgatgttgttgttagttggggtggacatgatgcaattaaatgggcaacagaacatacaccagtaaacgtcgacatattaaaatttgggccgaagaaaagtattgcgattgttgatgatcctgtagatattacagcttctgctattggcgtcgctcatgatatttgtttttatgatcagcaggcctgtttttcaacccaagatatctattatataggcgataacattgatgcgttttttgatgagcttgtagaacaattagatatatatatggagatattaccaaaaggcgatcaaacatttgatgaaaaggcatcattttcattaattgaaaaagagtgtcaattcgcaaaatataaagttgaaaaaggtgataatcaatcttggttattggttaaatcaccgctaggatcttttggtaatcaaccattagctagatctacatatattcatcacgtctcggatatatcagaaataacgccttatatagaaaacagaattactcaaactgtaactgttactccttgggagtcatcatttaaatatagagatgttctagcctctcatggtgcagagcgtattgttgaatctgggatgaataatatcttccgtgtcggtggtgcgcatgatggtatgaggcctcttcaacgtttagttaaatatatttcacatgaaagaccttctacatatacaaccaaagatgtggcagtaaaaatcgaacaaacacgttacctagaagaagataagtttttagtctttgtaccataaaaaggaattaatatgaaaagtgaaaacaattctgtgccaattgatcatgttataaaagttgataatgaccaatatatacgtgtttgggaaacaatccctaaaaatcaaggtgataaaagaaataatactattgttattgcttctggttttgctcgaagaatggaccattttgcaggtttagcagaatatttatcgaccaatggatttcatgttattcggtatgattcacttaatcatgttggattaagtagcggtgaaattgatcagttctcaatgtcagtcggcaagaaaagtttattaaccgttattgattggttgaaatcagagcatggtattgatcaaattggtttaattgcatcaagcctttctgctcgaattgcttatgatattgttgctgatgttaatttgtcttttttaattaccgccgttggtgtggttaatttacgaaacactcttgaacaagcacttaaatatgattacttgaagatggaaattgatgaaataccagaagatctaaattttgatggatataatttaggttcaaaagtatttgttacagattgctttgaaaataactgggatacattagattcaactataaataaaacgaagaatttaaatttcccttttatcgcttttgtcgccaatgatgacagttgggtacaacagcacgaagttgaagaattaatgaataatattaattcagataaaaccaagatttactctttaataggttcatcacatgatttaggtgaaaatctaatagtgctaagaaatttctatcaatcaattacgaaagctgcgattgcattagatagtaatttattagggttagcgagtgagattgttgagccacaatttgaagctcttacaattgctacagtaaatgaacgccgcttgaaaaacacaataaaaagtaagtcattagtttaattacaactgatacataaaccaaccaaaggaatatattatgaaatttggaaatatttgtttctcatatcagcccccaggtgagtcacataaacaagtcatggatcgctttgttcgtttaggtgttgcatcagaagagctaaattttaatacttactggactctagagcatcattttactgaatttggtctaacaggtaacctttttgttgcttgtgctaacttacttggtcgaaccacaaaattacatgttggcacaatgggaattgtacttcctactgctcaccctgcgcgtcaaatggaagacttactacttttagatcaaatgtcaaaaggtcgttttaattttggtgttgtacgtggcttgtaccataaagattttcgcgtctttggtgtaacaatggaagattctcgtgccattactgaagattttcataccatgattatggatggcacaaaaacgggtacacttcatactgatgggaaaaacatcgagttcccagatgttaacgtttatccagaggcgtatttagataaaattccaacatgtatgactgcggaatcagcagtaacaacgacttggcttgctgagcgtggtttaccgatggtgcttagctggattattacaaccagtgaaaagaaagctcaaatggaactctataatgctgttgctagagatagtggttacagtgaagagtatattaaaaacgttgatcacagtatgaccctcatctgttctgtagatgaagatgctaaaaaagctgaagatgtatgccgtgagtttttgggaaattggtatgactcatacgtaaatgcgaccaatatctttagtgaaagtaaccagactcgtggttatgattatcataaaggtcaatggaaagatttcgttcttcagggacatactaatacgaaacgtcgagttgactatagccacgatttaaaccctgtcggtacacctgaaaaatgtattgaaattattcaacgtgatattgatgcaacaggtattactaatattacccttggttttgaagcgaatggttcagaggaagaaatcattgcctctatgaaacgtttcatgacacaagttgcaccattcttaaaagatccaaaataaatcacttagattaacgttaataaataatataaggaatataacatgaattttggattattcttcctcaactttcagcctgaaaacacatcgtcagaaactgttttagataatatgatcaatatggtgtctctggttgataaagattataaaaactttacaactattttagtcaatgagcaccatttttctaaaaatggtattgtcggtgccccgatcactgctgcgagcttcctattagggttaactgaacgtttacacattggttctttaaatcaagttattacaactcatcacccggttcgtattgcagaagaagcaagtttacttgaccaaatgtcagatagtcgttttattctaggtttaagtgattgtattaatgattttgagatggatttctttaaacgtcagcgtgattcacagcagctacaatttgaagcttgttatgagatcattaatgaagcaatcacaaccaattattgccaagcgaataatgatttttataacttccctcgcatctcaattaatcctcattgcctgagtaaagagaatatgaagcaatatattttagcttctagtgtgagtgttgttgagtgggcggctaaaaaagcgctgccactgacatatcgttggagcgatagccttgaagataaagagattctttataagcgttatttagacgttgcagcaaatcataatattgacgtttctaatgtcgagcatcagttcccactgcttgtaaatttaaatcatgatcgtgatgttgctcatcaagaagcaacaacctatttagcaagttatattgccgaggtatatccgcatctaaatcagcaacaaaaaatggctgaacttattagccaacatgcgattggtactgataatgattactatgaatcaacattaaatgcgttagagcgtacaggttcaaaaaatgtattactttcttttgaatcaatgaaaaatcatgatgatgttgtaaacgtgattaatatggtcaatgagaaaattcaaaagaatttaccaagctcgtaaacataaaggtggcggtgttattatgaataaatggaattacggaattttcttcgttaacttttatagtaaaggcgaacaagagtcatcaaaaatgatgaataatgcgttagaaacattacgcattattgatgaagatacatctatttatgatgtggttaatattaatgatcactatcttgtaaagaaagatagtgaagataataagttagcgccttttattgcactagggtctaaattatatgtgcttgctaccagtgaaaacacagttgatagcgcagcaaaatatgcattaccgctagtttttaaatgggatgatacaaacgaggaacgacttaaattattgagttcctataatacatccgcaagtaaatataatcagaatatagattcggttcgacaccagcttatgttacatgtcaatgttaatgaggcagaagctgtagcaaaagaagagctaaaattatattttgaaaactatgtagcatgtacacaacctagtaattttaatggctcgattgatagtattattcagagtaatgtgacagggtgttataacgattgtttgtcatatgtagcgaatcttgctggtaaatttaataatactgtggacttcttactttgctttgagtcaatgcaagataaaaataagaaaaaatcagtaatgatagaacttaataatcaagttattaagtttcgccaagataacaatctaatctaatctacaatcattgccgcttataatggcagtgctaatttaaagttctgccattatatttaattatatcttaaataggattaaacatgactattatattagatacttgcgaaaaagatattattgtaagttcagagatcgacgatattatttttacatcctcacctcttgatattacttatgatgaacaagaaagaataaagcataaattaatattagaatcatttcgttatcattataacaataatgaagattataagtttttctgtaatgctcagggggttgacggaaatatttcatcacttgacgatatccctgtttttccgacttcaatgtttaagtatgcaaaaatatgtacagcagatgagtctaacattgaagactggtttacaagtagtggtactagtggtgtaaaaagttatattgctcgtgatcgtgtaagtattgaacgtttactgggttctgtaaattatggaatgaaatatcttggctcatttcatgaaaatcagttagagcttgttaatatggggcccgatcgttttaatgctaaaaatgtttggtttaagtatgtaatgagtcttgttgaattattatatccaaccacatttacggtgaataatgatgaaatagattttgaacttaccattaaaagtttaaaagaaatctataataaaggtaaaggcatttgtctcattggtcctccatattttatttacttattatgccagtacatgaaagagaatgatattgagtttaatgctggtaatcgaatctttattattactggtggtggttggaaaactaagcaaaaacaagcgctaaaccgtcaagattttaatcaactattgatggataccttccacttagcacatgaaagtcagattcgagatacatttaatcaagttgaattaaatacctgtttctttgaagataatcgtcagcgtaagcatgttccgccatgggtttatgcacgtgcacttgatcctgtgacactaaaacctgttgaagatggtcaagagggtcttattagttatatggatgcatcatcaacgagttacccaacatttatcgttactgatgatatcggtattattcatactattaaagcgccagatccactccaaggtactacgattgatatcgtccgccgtttgaataccagagaacaaaaagggtgttcattatcaatgtcatcaggtttaaaatagatcataaggaagatgtatgattttaaattgtaaaataattaaaattgaagcttctgaatgtaatatttttaaagtatttattaagcctgataagtgtctcaatttcaaagctgggcaatatgttttagcgtatttagatggtaaaaaattacctttttcaattgctaattgtccaacatgtaatgagcttatagagttacatgttggaagttcggtaaaagaaacagcagttaaatctatttcttactttgtagatgcttttgtgaatagcgatgacatacaaatagatgcacctcatggtaatgcttggttacgtgagggcagtaattcgccattattacttattgctggaggtacaggactatcatatatcaatagtattcttagtaattgtgtaaataggaatttacctcgttctatttatgtttactggggagttaataatattgatttattatatgcagacactcaattaaaaacactttctagcgactttagtaatgttaaatacgtgcctgttctagaaaactttgataatagttggtatggaaaaaaaggtaatgttattgatgcaataatagaagatttttgtgatttatcagattttgatatttatgtttgcggccctcaaggcatgacttatagtgttcgagaaaaattaacatcacttaaaaaagcgaatgctgataaaatgtttgctgatgcttttgcatatatgtgatcttaatttaagttaataaaaattaaaaaaactttaaattcgctatgaggttatatttatatgaattgatttttaactctattttttggtttgtaatattattattctctcaataaatagagttattactaaatttgtaattaacgttaagttcaagtttttgatatgtttgttagggattttgaaaaatattaataagatattacctgtaacttcagtaatttttattcaataggaaataataaaggaatgattatgacgttaagtacagcccaagaaatcattgaggatattcgccaagggaaaatggttatattaatggatgatgaggatcgtgaaaatgagggcgatcttattattgcatcagataaaatcacgcctgaagcaataaattttatggcaacttacggtcgtggtttaatttgtctgacattaaacaaagcccgttgcctgcaattaaaattacctttgatggtgaagaacaataccgataaatttgcaaccccgtttactctttctatagaagcggcttctggggttacaaccggtatttcagtaaaagatagagcgcgcactgttcaagcggctgtagcggcaatggcgacatcggaagatattgttatgcctggacacatttttccattaatggctcaagatggcggtgtattaactcgcgcaggccatactgaagctggttgtgatgtcgcgcgattagcaggattagagccatccagtgttattgttgaaatattgaatgatgacggtacgatggcgagacggccgcagttagaagtctttgctaataagcatggcttaaggttaggtactgtcgctgatcttattgaatatcgaaataaatatgaaaccatgattgaacgtatttctgagtgtaaattgaagactgaatatggtgaattcaatatgatcacttatcgagataaaattaatcatcaaattcattatgcgctacaaaaaggtaatattgagccgaattctcaaaccttagtgcgagtgcatttacaagatacatttaaagatattctgcaaacaggatcgaatcgatggacattacccgccgcgatgagtcgtattagttctgaaaatggcgttcttgttatagtaactaaaccagaagatcctgaaattgtaatcagtaaaattcagaatctagctttgggtaatcaagaaacgccagtgattaatagtcaatcacgtcaggttggattaggttcgcaaatattatcagatcttggcgttagaaaaatgcgtttattatcatctagtcgtcagctttatcattcattatctggtttcggtcttgaaatagttgagtatgtgtgtgattaagtttcgatacagtaataagactagccgttatttatactaaaattaattataaatattataggagtacccatgaagctaattgaaggtgccaccgtagcacctaatgctaaagttgctattgtaattgcacgttttaatagttttattaatgacagtttattatctggcgcgcttgatgcgttgcaacgtcaaggtcaagttagcgatgataatattactataattcgttgccctggagcttatgagttacctcttgttgcccagtttacggccaaaactgatcgttatgatgcaattatagctttaggtgctgttattcgaggtggtacaccgcattttgaatatgtggctggtgaatgtaataaaggtcttgcgcaagtcgcattagattataatattccagttgcttttggtgtgttgactgttgattcaattgaacaagcgattgaacgtgctggcactaaagcgggaaataaaggtgcagaggctgcattaagtgtacttgagatggttaatgttttggctcaagttgaatcttaactatataacggtttattaaaattaagttacgagtgtttaattacactcgtttataaatacaataccgaatagttattaagtaatgaatattagtcatattcgttgattaagtttgttgttatcgaaagagaataaatacttctttatttcacatggaaatatttaggaatattatggtcaatgttagggaaagagtacctttaaacgtgggtattaatagtgatattcctgccgagttgctttcgtttaatggtcttgaatcgggaaaagaacatatagcacttatttttaaagaagcagataaaatattggttcctttagttcgtatgcattctgagtgtttaacgggcgatgtttttcattcatcacgctgtgattgtggagagcagttagttgaaactattgaaaaaatgactgagcaaggtggtattattttatatttgcgtcaggaaggtcgcggtattgggctctataataagatcgatgcttataagctacaaagtcaaggaatgaatacttatgaagcgaataattatttaggttttgatgacgacttacgagagttttctgaagcagctcaaatgcttactgctcttggtattcagaatatacatttagtgacgaacaatcctaagaaaatttttgatttacaacaaaacggtataaatattgtagaagttgttggaactaacgttcattt

SEQ ID NO: 8 - DNA, 1065 bp, Photobacterium leiognathi
atgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaa

SEQ ID NO: 9 - DNA, 981 bp, Photobacterium leiognathi
atgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataa

SEQ ID NO: 10 - DNA, 1437 bp, Photobacterium leiognathi
atgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtag

SEQ ID NO: 11 - DNA, 948 bp, Photobacterium leiognathi
atggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaa

SEQ ID NO: 12 - DNA, 1122 bp, Photobacterium leiognathi
atgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataa

SEQ ID NO: 13 - DNA, 705 bp, Photobacterium leiognathi
atgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaa

SEQ ID NO: 14 - DNA, 745 bp, Vibrio fischeri
ataaaggattaagttatgattgttgatggcagagtttcaaagatagttttagcatcgataaaaaataatatatataaggtatttattactgtaaattcaccaataaagttcatcgctggacaatttgtaatggtcacgattaatgggaaaaaatgtcctttttcaattgcgaattgcccgacaaaaaatcacgaaatagaattgcatattggtagttcgaataaagactgctcattggatattatcgaatattttgtcgatgctcttgttgaggaagtcgcaattgagttagatgctccccatggaaacgcttggttacggtctgaaagtaataacccattgctattaattgcgggaggtacaggtttatcatatataaatagcattctaaccaattgcttaaatcggaatatacctcaagatatttatctttactggggagtaaaaaacagttctcttttgtatgaagacgaagagttattggaattatcactaaataacaaaaatcttcattatattcctgttatcgaagataaaagtgaagaatggataggaaaaaaaggtacggttcttgatgctgtaatggaagattttacggatctagcccattttgatatttatgtttgtgggcccttcatgatggctaaaacagcaaaagaaaaattaattgaagagaaaaaagcaaagtcagaacagatgtttgccgatgcttttgcatacgtataaagagaatataaaaagccag

SEQ ID NO: 15 - DNA, 705 bp, Photobacterium leiognathi; identical to SEQ ID NO: 13

SEQ ID NO: 16 - DNA, 705 bp, Photobacterium phosphoreum
atgattttaaattgtaaaataattaaaattgaagcttctgaatgtaatatttttaaagtatttattaagcctgataagtgtctcaatttcaaagctgggcaatatgttttagcgtatttagatggtaaaaaattacctttttcaattgctaattgtccaacatgtaatgagcttatagagttacatgttggaagttcggtaaaagaaacagcagttaaatctatttcttactttgtagatgcttttgtgaatagcgatgacatacaaatagatgcacctcatggtaatgcttggttacgtgagggcagtaattcgccattattacttattgctggaggtacaggactatcatatatcaatagtattcttagtaattgtgtaaataggaatttacctcgttctatttatgtttactggggagttaataatattgatttattatatgcagacactcaattaaaaacactttctagcgactttagtaatgttaaatacgtgcctgttctagaaaactttgataatagttggtatggaaaaaaaggtaatgttattgatgcaataatagaagatttttgtgatttatcagattttgatatttatgtttgcggccctcaaggcatgacttatagtgttcgagaaaaattaacatcacttaaaaaagcgaatgctgataaaatgtttgctgatgcttttgcatatatgtga

SEQ ID NO: 17 - DNA, 702 bp, Photobacterium phosphoreum
atgttatgttcgatagaaaaaattgagccgttaactaacctcatattccgagtattgctcaagccagatcagccttttgaatttagggcagggcagtacattaacgtcagtttaagctttggtagtttaccgttttctatagcctcatgtccttctaatggtgcgtttttagaactccatattggtggctcagatatcagcaagaaaaatacgcttgtgatggaagaactcaccaattcatggggctgcggcaacatggttgaagtcagtgaggcgcgaggtgaggcttggttgcgtgatgagagtgtcaaacccttgttattggtcgcaggcgggacgggaatgtcatacaccctaagtattttgaaaaatagcttggagcaagggtttacccagccgatttacgtctattggggcgccaaggatatggataacctgtatgtacatgacgaactggtggatattgcgcttgaaaacaaaaacgtcagttacgtgccagtcactgaaatatcaacctgtccccaatacgctaagcaaggaaaggtgttggagtgtgtgatgagtgatttccgtaacttatctgagttcgatatctacttgtgtggtccttgcaaaatggttgaagtggctcgtgattggttctgtgacaaaagaggggcagaaccagagcaactttacgcggacgcgttcgcttatttgtaa

SEQ ID NO: 18 - DNA, 711 bp, Vibrio fischeri
atgattgttgatggcagagtttcaaagatagttttagcatcgataaaaaataatatatataaggtatttattactgtaaattcaccaataaagttcatcgctggacaatttgtaatggtcacgattaatgggaaaaaatgtcctttttcaattgcgaattgcccgacaaaaaatcacgaaatagaattgcatattggtagttcgaataaagactgctcattggatattatcgaatattttgtcgatgctcttgttgaggaagtcgcaattgagttagatgctccccatggaaacgcttggttacggtctgaaagtaataacccattgctattaattgcgggaggtacaggtttatcatatataaatagcattctaaccaattgcttaaatcggaatatacctcaagatatttatctttactggggagtaaaaaacagttctcttttgtatgaagacgaagagttattggaattatcactaaataacaaaaatcttcattatattcctgttatcgaagataaaagtgaagaatggataggaaaaaaaggtacggttcttgatgctgtaatggaagattttacggatctagcccattttgatatttatgtttgtgggcccttcatgatggctaaaacagcaaaagaaaaattaattgaagagaaaaaagcaaagtcagaacagatgtttgccgatgcttttgcatacgtataa

SEQ ID NO: 19 - DNA, 711 bp, Shewanella hanedai
atgaaagtaaaatgcagcgtatcaaaaatagagttaataaataaaaatatatataaagtttatattaaaccttatgttcctattgatttcaaagctgggcagtatatatatataaatttaagtggtaataaaaggcaaccattttctattgctagttgtccaacagataatagtgtgattgaactgcatgttggcagttcaaatgaaaacagttctttagatgtaatggaatattttggtgatgctctaataaaaaatagtaccatcgtgatagatgctccacatggagaggcgtggttacgtgaaggcagtaataaaccaatattgttgattgccggtggtacaggtttatcctatattagcagtattcttcgaaactgtttaaaccgaggtttcactcaacctatttatgtttattggggagtgaaaaatatagattttttgtatgcagatgaagaactacagttattatgcagtcaccacagtaatttacattacattccagtggtattagaagacagtaaatatacgtggttaggaaaaaaaggcacggttattgatgctgtcatggatgattttactgttcttacgctttttgatatttatgtctgtgggcctaatttgatgactaaggcagcaaaagataaattagttgcaaaaaagagtgctaaatcagaacaaatgttttccgatgcttttgcatatatgtga

SEQ ID NO: 20 - protein, 66 aa, Artificial Sequence
  Synthetic amino acid sequence spinach lumazine synthase
MASFAASQTCFLTTNPTCLKPNSPQKSSTFLPFSAPLSSSSSFPGCGLVHVASNKKNRASFVVTNA

SEQ ID NO: 21 - protein, 32 aa, Artificial Sequence
  Synthetic amino acid sequence Chlamydomonas ferredoxin
MAMAMRSTFAARVGAKPAVRGARPASRMSCMA

SEQ ID NO: 22 - protein, 32 aa, Artificial Sequence
  Synthetic amino acid sequence Rubisco activase
MQVTMKSSAVSGQRVGGARVATRSVRRAQLQV

SEQ ID NO: 23 - DNA, 4553 bp, Artificial Sequence
  Synthetic nucleotide sequence pSAT1-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccggcgcgccaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcggcgcgccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 24 - DNA, 4560 bp, Artificial Sequence
  Synthetic nucleotide sequence pSAT2-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccgctaccttaagagagaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcggcgcgccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 4567 bp, Artificial Sequence
  Synthetic nucleotide sequence pSAT3-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccgctaccttaagagagaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcgctaccttaagagaggtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - DNA, 4573 bp, Artificial Sequence
  Synthetic nucleotide sequence pSAT4-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 27 - DNA, 4589 bp, Artificial Sequence
  Synthetic nucleotide sequence pSAT5-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcctcgctaccttaggaccgttatagttaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgctcgctaccttaggaccgttatagttagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 4597 bp, Artificial Sequence
  Synthetic nucleotide sequence pSAT6-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacccataatacccataatagctgtttgccaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcacccataatacccataatagctgtttgccagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 29 - DNA, 4599 bp, Artificial Sequence
  Synthetic nucleotide sequence pSAT7-EGFP-C1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcccataaagccgtcagtgtccgcataaagaaccaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccacctagtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgccataaagccgtcagtgtccgcataaagaaccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - DNA, 150 bp, Artificial Sequence
  Synthetic nucleotide sequence Shimizu et al.
gctctagttggatttgctcccccgccgtcgttcaatgagaatggataagaggctcgtgggattgacgtgagggggcagggatggctatatttctgggagcgaactccgggcgaatttgaagcgcttggatacagttgtagggagggatcc

SEQ ID NO: 31 - DNA, 150 bp, Artificial Sequence
  Synthetic nucleotide sequence Lutz et al.
tatagatccgctcccccgccgtcgttcaatgagaatggataagaggctcgtgggattgacgtgagggggcagggatggctatatttctgggagtcgagtagaccttgttgttgtgaaaattcttaattcatgagttgtagggagggattt

SEQ ID NO: 32 - DNA, 95 bp, Artificial Sequence
  Synthetic nucleotide sequence truncated Prrn promoter
cgccgtcgttcaatgagaatggataagaggctcgtgggattgacgtgagggggcagggatggctatatttctgggagcgaactccgggcgaatat

SEQ ID NO: 33 - DNA, 95 bp, Artificial Sequence
  Synthetic nucleotide sequence truncated Prrn promoter
cgccgtcgttcaatgtgaatggataagaggctcgtgggattgacgtgagggggcagggatggctatatttctgggagcgaactccgggcgaatat

SEQ ID NO: 34 - DNA, 95 bp, Artificial Sequence
  Synthetic nucleotide sequence truncated Prrn promoter
cgccgtcgttcaatgagaatggataagaggctggtgggattgacgtgagggggcagggatggctatatttctgggagcgaactccgggcgaatat

SEQ ID NO: 35 - DNA, 95 bp, Artificial Sequence
  Synthetic nucleotide sequence truncated Prrn promoter
cgccgtcgttcaatgagaatggataagaggctcgtgggattgacgtgagggggcagggatggctatatttctgggagcgaactgcgggcgaatat

SEQ ID NO: 36 - DNA, 95 bp, Artificial Sequence
  Synthetic nucleotide sequence truncated Prrn promoter
cgcggtcgttcaatgagaatggataagaggctcgtgggattgacgtgagggggcagggatggctatatttctgggagcgaactccgggcgaatat

SEQ ID NO: 37 - DNA, 95 bp, Artificial Sequence
  Synthetic nucleotide sequence truncated Prrn promoter
cgccgacgttcaatgagaatggataagaggctcgtgggattgacgtgagggggcagggatggctatatttctgggagcgaactccgggcgaatat

SEQ ID NO: 38 - DNA, 95 bp, Artificial Sequence
  Synthetic nucleotide sequence truncated Prrn promoter
cgccgtcgttcaatgagaatggataagaggctcgtgggattgacgtgagggggcagggatggctatatttctgcgagcgaactccgggcgaatat

SEQ ID NO: 39 - DNA, 95 bp, Artificial Sequence
  Synthetic nucleotide sequence truncated Prrn promoter
cgccgtcgttcaatgagaatggataagaggctcgtgggattgacgtgagggggcacggatggctatatttctgggagcgaactccgggcgaatat

SEQ ID NO: 40 - DNA, 95 bp, Artificial Sequence
  Synthetic nucleotide sequence truncated Prrn promoter
cggcgtcgttcaatgagaatggataagaggctcgtgggattgacgtgagggggcagggatggctatatttctgggagcgaactccgggcgaatat

SEQ ID NO: 41 - DNA, 95 bp, Artificial Sequence
  Synthetic nucleotide sequence truncated Prrn promoter
cgccgtcgttcaatgagaatggataagaggctcgtgggattgacgtgagggggcagggatggctatatttctgggagcgaactccgggcgattat

SEQ ID NO: 42 - DNA, 95 bp, Artificial Sequence
  Synthetic nucleotide sequence truncated Prrn promoter
cgccgtcgttcaatgagaatggataagaggctcgtgggattgacgtgagggggcagggatcgctatatttctgggagcgaactccgggcgaatat

SEQ ID NO: 43 - DNA, 119 bp, Artificial Sequence
  Synthetic nucleotide sequence truncated Prrn promoter with leader
cgccgtcgttcaatgagaatggataagaggctcgtgggattgacgtgagggggcagggatggctatatttctgggagcgaactccgggcgaatatccatggagttgtagggagggattt

SEQ ID NO: 44 - DNA, 21 bp, Artificial Sequence
  Synthetic nucleotide sequence GeneBank # DQ882177
agttgtagggagggatttatg

SEQ ID NO: 45 - DNA, 395 bp, Artificial Sequence
  Synthetic nucleotide sequence
gatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcga

SEQ ID NO: 46 - DNA, 161 bp, Artificial Sequence
  Synthetic nucleotide sequence
accgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtgttct

SEQ ID NO: 47 - DNA, 678 bp, Artificial Sequence
  Synthetic nucleotide sequence DsRed
atggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaagactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccagtag

SEQ ID NO: 48 - DNA, 3490 bp, Photobacterium leiognathi
agggatagtatgtttacaggaataatagaagcagtgggaaatatcagcgccattattcgtaataacgacgatatttcaatggttatcaataccaatcggcttgatatttcggatgtaaaacttggcgatagcattgctagcaatggcgtttgtttaaccgtatctaaattaacgccaactggatttgttgccgatctttcaacagagacattaaagcgtaccgcttttcacagttaccacgttggacaaaaaatcaatttagaaaaagccatgttacccaccacacgctttggtggtcatatcgtttctgggcatgttgatggtattggcgacattattgagctaaagcgaaaaggtcgcacccttgatatgtggataaccgtgcctattcacctaaagaaattcgtttccgagaaaggttcagtttgcattgatggaaccagtttaaccatcaacgctgtgtatcagaatgtcattaaattaaccataatccctcatacgttagctaacacgacacttgccaatgccaccattgatcaaaaagtcaatattgaagccgacatgatggcgcgctacctcgaacgattaatcagtattgataaacaagaatctaaaaaaaacactaacgtctctatgtctttgttagagaagcacggctttatcgtataactcacttacactgttgataataaaggacccattatgactttaagtagcgcccaagaaatcattaatgatatccgtctgggtaaaatggtgatcttaatggatgatgaaaatcgcgaaaatgaaggcgatttgatcatcgccgctgacatgatcacccctgaagcgattaatttcatggcgacctatggacgcgggttgatctgcttaacgttaaccaaagatcgctgccagaccttaaatttaccgcttatggtgcaaaataatcacgataagttctcaactgcatttaccgtttcaatagaagccgccgccgatgttacgaccgggatctctgcctatgatcgtgcaaaaacagtacaagccgccattggcccaaatgccagtgctgatgatattgtgatgccaggtcatatttttcccttaatggcgcaagatggtggcgtattaattcgtgccggccacactgaagctggctgtgatgttgcacgtttagctggacttgaagcctctagtgttattgtggaaattcttaatgaagacggcacgatggcacgccgacctcaactcgaagtctttgctcaaaaacacggtttaaaactgggaacgattgctgatttaattgagtatcgtacccaacgagaaagccatattgaacgtttatcagaaagtgaattatgcactgagtatggcgtttttaacttaatcacctaccgtgacaccattgataaccaactgcattacgcattatgtaaaggtgatataaaccctgatagtgagaccttagtccgtgttcatgttaaagatacactgaaagatattttacatactggcgcaacgcaatggtctctacaagcggccatgcagcgcattcaagccgacggtggcatattagtgatcataagccaaattgagccctcagcgatgatcctaaatcaaataaaccacttagattcagagcaccaagcgagtttacctttacctatcacaccgcaatcccgacaaattggtttaggatcgcaaattctgtctgaattagggctatgcaaaattcgtttactgtcttctcagagccaacaatatcgttctttatcaggatttgatcttgaggttgtggaatatatttgtaactagtaaacacttagatattattgattcatcattcttttttacaaatacacttaggaagctccatgaatattattgaaggtgcaatcacagcaccccatgccaatgtcgcgatcatcgtttctcgtttcaatagttttatcaatgacagcttattgtctggtgcattagatgccctacaacgccaaggtcttgttaaagaaagtaatattaccgttgtacgttgccctggagcatacgagctacctttactggcgcaacaactggcaaaaaaaggcagttatgatgccattatcgctttaggctcagtgatacgtggcggtactccacattttgaatatgttgcaggtgaatgtaataaaggtttagcgcaaattgcgttagagcatcaaattccagtcgcgttcggtgtccttactgtggattcaatcgaacaagccattgaacgtgcaggaacaaaaatgggaaataaaggtgcagaagcggcattaagtgcactagaaatgattaacgtgctagcagaaatcgagccttaatttccttatattttcacccgtcatgatggctatgatgccctacgtcatcatagccattgcccttctaaagcaatgaactcaaatagaattattaggaatattatgatcacggtaagagcccgggtgccttttaaaatcgggcaaaaaagtcagatcccagcggagttattgtcgtttcatggctttaattcaggtaaagagcatatcgcaattatttttaaacaagcagataccacaaccgcccctttagttcgcttacattcagaatgcttaaccggtgatgtctttcattcttcgcgctgtgattgtggcgagcagctcaatgaaacaatagaaaaaatgcatcaattcggtgggatcattttgtatctacgtcaagaaggacgtggcattggtttatacaataaaatcgatgcctatcacttacaaagccaaggcatgaatacctatgaagccaataaccatttaggtttcgctgatgatctacgcgattttagtgaagctgcaaaagcattaatggcattaggcataaacaagatccgcttaatcactaacaacccaaagaaaatcaatgaacttcaacaacatggcattgagatcatagatgtggttaacactcaggtacacatcaaacaagacaacgaagattatctaaaaacaaaagcgttacacggccatcacaacctctctttatgatcagatctgctcaacgcttatattgagctatcttagccagaagattaactcttaccaatattgcgccatttaacagtactcccccttatgttaaatggcgaaaaaacaaccgccctcaaaaatccacagctaagagttacccactaaatttgtggataacacaatattacctttatttttcaacatattatgtttttcaatagaaatacataataatctcgactattttcaaaaatataaatacgtccttaaaacgtaaaaagccgcactattgcagtgcggcttggtgttgctattagtgtacttactcggcacaccaatattgttacgcttcgccactcattacgttgagcttgccgtgttcgacatagtaggttcgaaccgctatttattggcgaagaagcaaatttttaaaaggtgtcacttaccaaattgttaacaactaagttttcacttagaagctt

SEQ ID NO: 49 - DNA, 1133 bp, Photorhabdus luminescens
gtcgacaaaatctgggatgaattagatattttaggaccataagagggaacgcatgacaacactgagctgtaaagtaacctctgtagaggctattactgatacggtttatcgggtacggttgcttcccgattctccgttcttattccgcgccggtcagtatctgatggtggtaatggatgagagagataaacgtccgttttcaatggcgtcaacgccttcagaaaaggagtttattgaattacatattggtgcttctgaactgaatttgtatgcaatggctgtgatggatagaattctggatcagaaagtgatcaatattgatatccctcatggcaaagcttggttccgtaaaagcagcgctaatccgttgttattaattgctggcggtacggggttttcttacacccgttcaatattattgacagcgttggaagaacaaccaaaacgtcatatctctatgtattgggggggcagagaatcacaacatttatatgatcttgctgaattacggttacttacagaacgctatcctaatttgaaggttattccagttgttgaacagtcagataatggttggtgtggacgtacaggaacagtgcttaaagcagtactagaggattttggtagtttggccaattatgatatctacattgcagggcgattcgaaatggcaaaaattgctcgcgagcgcttttgtagtgagcgtgatgcttctgctgacagcatgtatggtgatgctttcgaattcatttagaataataaaaaaacccgcccctgacaggcgggaattacggcaacaacgactcagttataataattcttatatacccgtcatctttcaagttgcctctttgttggctgcactcactcaccccggttacatagttttctatgctcctggggattcattcacttgccgccgcgctgcaactcgaaatctattaggtatagataagttcttaatccattctttctataatggtggcgataccttggcctaaaccgatacacatggttgctaggccaaactgaacatcgcggcgttccattaagttcaacaacgttgttgtgatgcgagcgcctgagcagcctaaaggatgacccagagcaattgcgccaccattcaggttaac

SEQ ID NO: 50 - DNA, 1121 bp, Vibrio fischeri
ctcgaggcgggaattaattatccaaaccgatgccaagtcggcgcatgtgctatgtgcttatgcaaaaaattagagggtgaaattgaatacgatttagagcctcttcttaccgataaagaacaacaagaagggtgggtatttgcgtgtcaggcaacagcaaaaagtgatttagtgctgttgttagaataaatcctccccgtataattagagtttaatgctcaatacacataataatgacagcgtacaaatgccatattaaaaaggcatcagctgaaaaaggaaagtcatgccaatcaattgcaaagtaaagtctatcgagccattggcttgtaatacttttcgaattttacttcacccagaacagcctgttgcttttaaagcaggccaatacctaacggttgttatgggtgaaaaagacaaacgcccattctcaatcgcaagtagtccttgtcgccacgaaggtgaaattgagttacatattggtgccgcagagcacaatgcttatgccggagaagtggttgaatcaatgaaatcggcactagaaacgggtggtgatattttaattgatgcgcctcatggtgaagcgtggatccgtgaagacagcgatcgttcaatgttattgattgctggcggtacaggttttagttacgtacgttcaattcttgatcactgtattagccaacagattcaaaaaccaatttacctatactggggtggtcgtgatgaatgccaactgtatgcaaaagcagaattagagagcattgctcaagcgcatagccatattacgtttgtgccagtggttgagaaaagtgaaggctggacaggtaaaacgggtaatgtgttagaagcggtaaaagccgattttaactcactagcagatatggatatttacatcgcaggtcgctttgaaatggctggtgcagcacgtgagcagttcaccactgaaaaacaagcgaagaaagagcagctgtttggtgatgcattcgcatttatctaatttagagcactaaaaagacaaataaaaatgccactcaataatgagtggcatttttttatggatgttataaaaaatgaattagcctttatcatcaaccatagtcagtgctttacgagaaagatct

SEQ ID NO: 51 - DNA, 702 bp, Escherichia coli
atgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatctga

SEQ ID NO: 52 - DNA, 2218 bp, Arabidopsis thaliana
cgtcttctccgcccttcttattcatttctactctgctgcgttatttctccgtcgtcctcaactcatcgccggcgtttaccatccgctagatccgtcgcctctccggtcgactatttatatggaatcgtaaatttacagacactaataagaatagtttgacaaaatctgtagctggaataatgtcgatgagcaattccttgaaaaagctctcttcttgtgttcttattgatctcgatggtaccttaatcaacacaggtaacttaaaacctcaatagattgtttcaatttctgtttatatagcttggaattcgaatttgatgtttttttaaaattcattgtagatggtgttgttggtgacattttgaggaaatacttgtgtaaatatggtaaacagtgggatggaagagaatcattgaaaatagttggcaaaactccagtagaagctgctactactattgttgaagattatgaacttccttgtaaagttgatgaattcaattctgaattctaccctttgttctctgctcagtaagttaccataaaaaaaatcgatctttgtccttattgttgtaatcttcatattgttgattacctccggttttgtaggatggacaaaatcaaatctcttcctggtgccaataggttgattcgacatttgaagtgtcatggagtacctgtggctttggcttccaattcttcaagagcaaatattgaatccaaaatttcatatcatgaaggtttgttttatatgaaatgttgttactcaatgcataagcttgtatagaggtaaacttgttttatgaaaatggtttcacttttgtgtagggtggaaggaatgcttctcggttattgttggtagtgatgaagtctctaaaggaaagccttctcctgatatgtaagatccatctcttgtcacccatttgtggtttcatctattgattccaattaaacaatgtctggttaatgacaatgatcatatttgtaaaaagttttctcgaagcagcgaaaagattgaagaaagatccagctgactgtttggttattgaagattctgtgtaagtcttacttctagagtatcgttttgctgttgtgtgtttgattcattctcactaatagttgtttgaaatggtaggcctggtgttatggctggtaaagctgctgggacaaaagtgattgctgttccttctctgcctaagcaaacacatctttatacatctgcagatgaagtcatcaattctctactcgacataagacttgaaaaatggggacttcctccattccaagactgtaaaaattcactcatgaactctgttcaatactcctcttccttgataacatgttcatctcggtaatgagacttttataccttgtaatttgtatttgcagggatagagaacactttaccaattgatccatggcatattggaggtccagtcatcaaaggatttggccgtggctctaaagtactcggaatccccaccggtcagtgtacaaacctcattgaactcagctcttttacatcattatatatggcttcttttcttatcgctgcatttgttttgttttcagctaacttgtcaacaaaggattatgcggatgagctagtggaacatccttcgggagtgtactttggttgggcaggcttggcaaagagaggtgtctttaaaatggtcatgagcattggttggaatccttatttcaataacaaggagaaaactattgtgagttcataaaaagttcaaacaaaattcttactccagtggttgcttctttaactttagcttttgttatataggaaccatggctgcttcacgatttcactgaggatttctacggagaagagctacgtcttattatcgttggctatatacgccctgaggttattataacgttatcatcattcattcttcatatgactcctttgtgtaatgcaatattccttgtttgctcatattgtttgtgtttgctaggctaatttctcttcactggagagtctcattgcaaagattcacgaggacagggaagttgcagagaaagctcttgatcttccttcgtatgctaagtttaagggtgatccttatctgactaaatgatagaagcttttatgaaaagaattcaaacacttggatttgtaatttcataactcaaatcagacttgcattgttctttagtactatcagcctctttgatttgattaactagataacaactttcgtta

SEQ ID NO: 53 - DNA, 942 bp, Escherichia coli
atgaagctgatacgcggcatacataatctcagccaggccccgcaagaagggtgtgtgctgactattggtaatttcgacggcgtgcatcgcggtcatcgcgcgctgttacagggcttgcaggaagaagggcgcaagcgcaacttaccggtgatggtgatgctttttgaacctcaaccactggaactgtttgctaccgataaagccccggcacggctgacccggctgcgggaaaaactgcgttaccttgcagagtgtggcgttgattacgtgctgtgcgtgcgtttcgacaggcgtttcgcggcgttaaccgcgcaaaatttcatcagcgatcttctggtgaagcatttgcgcgtaaaatttcttgccgtaggtgatgatttccgctttggcgctggtcgtgaaggcgatttcttgttattacagaaagctggcatggaatacggcttcgatatcaccagtacgcaaactttttgcgaaggtggcgtgcgtatcagcagcaccgctgtgcgtcaggcgctggcggatgacaatctggctctggcagaaagtttactggggcacccgtttgccatctccgggcgtgtagtccacggtgatgaattagggcgcactataggtttcccgacggcgaatgtaccgctacgccgtcaggtttccccggtgaaaggggtttatgcggtagaagtgctgggcctcggtgaaaagccgttacccggcgtggcaaacattggaacacgcccaacggttgccggtattcgccagcagctggaagtgcatttgttagatgttgcaatggacctttacggtcgccatatacaagtagtgctgcgtaaaaaaatacgcaatgagcagcgatttgcgtcgctggacgaactgaaagcgcagattgcgcgtgatgaattaaccgcccgcgaattttttgggctaacaaaaccggcttaa

SEQ ID NO: 54 - DNA, 989 bp, Vibrio harveyi
gtttacgctcccaataaatgccgttatggtgaagattcagccaaatagaaccactcttcaggaagccagaacatcatgaacaatacgattgaaaccattcttgctcatcgctctatccgaaaattcaccgcagttcctattactgatgaacaaagacaaaccatcattcaagcaggtttagctgcgtcttcttctagtatgcttcaagtcgtctcaatcgttcgagtgactgactctgaaaagcgtaacgaattggctcaatttgctggtaaccaagcttatgttgaaagtgcggctgagttcttagtgttttgtattgattatcagcgccatgcaaccatcaatcctgatgtacaggcagactttacagaactaactctgattggagcagtagattctggaatcatggcacaaaactgcttgcttgcagccgagtctatgggattaggtggcgtatatattggaggactaaggaatagcgcagctcaagttgatgagctattgggcttaccggaaaatagcgcggtgttgtttggtatgtgcttagggcatcccgatcaaaatcccgaagtaaagccacgcctacctgcacatgtggttgttcatgaaaatcaataccaagagctaaatttagatgatattcagagctacgatcaaactatgcaagcgtattatgcgagccgtacaagcaatcaaaaactgagtacatggtcgcaagaagtcactgggaagcttgctggtgagtcgcgacctcatattctgccgtacttgaacagtaaggggctagcaaaacgctaatatcattgaaatgatggtttgttgtatgaaatcgttcatcaaaccatcactttgttgaacccacatcatattttgaccatacgctgccaactttctggcaaaaccataacaaagcgctattgactcagaacaaaaaacacgacatacatcacattttaaaacaaagcaatcactttgttgaacccacatca

SEQ ID NO: 55 - DNA, 6006 bp, Bacillus subtilis
aaaagtggacgaaccttatttggcggctaataaaaagagagcgaaacaggacgttttgaccatttgaccgatgatttcggcccggttaaagtgcctgataacaagtattttgtgatgggtgacaatcgtcgcaattccatggacagccgtaacggccttggcctcttcacgaaaaaacaaattgcgggtacgtcaaagtttgttttctacccgtttaacgaaatgcgcaaaacaaattaggatcaagcagcttcccattggggctgctttttttatatcttttttacggtcatcccctaaaaacagaacataaattcgtatatctatagaaaagaaatttttgcagaaatgtgaaacatattcccgttatgcatcgttatattaataatttacgagaatttacggttttttattcatgaaaaaaaggaataactcatatgaatgaatagattcatattggctggaggtttagaaatgggaagaataaaaaccaagattaccattctgttagtgcttttgcttttacttgcaggcggttatatgtacataaatgatattgagctgaaggatgttccgacagcaattggacaaaccttgtcctcggaagaagaggaatacaccatccaggaatataaagtgacgaaaattgacggctcagagtatcatggagtagcagaaaacggaacgaaaatcatcttcaacggaaaaaaattaaatcaggatttatctgatataaaagaaggtgacaagattaaggcttacttcagcaaatcaaagcggatcgacggattaatcaaggttgcaaaagtgaatgattaaaaaacatcactttcggatcgaagggtgatgttttgtttttctcaaattgtaagtttatttcattgcgtactttaaaaaggatcgctataataaccaataaggacaaatgaataaagattgtatccttcggggcagggtggaaatcccgaccggcggtagtaaagcacatttgctttagagcccgtgacccgtgtgcataagcacgcggtggattcagtttaagctgaagccgacagtgaaagtctggatgggagaaggatgatgagccgctatgcaaaatgtttaaaaatgcatagtgttatttcctattgcgtaaaatacctaaagccccgaattttttataaattcggggcttttttgacggtaaataacaaaagaggggagggaaacaaatggaagagtattatatgaagctggccttagatcttgcgaagcagggcgaaggacagaccgaatccaatccgctcgtcggcgctgttgtcgtaaaggacggacaaattgtcggaatgggcgcccatttaaaatatggtgaagctcatgcagaagttcatgccatccatatggctggagcacatgcagagggtgccgacatttacgttacactcgaaccgtgcagccattacggaaaaacaccgccatgtgcagaattgattatcaactctggtatcaaaagagtgttcgtggcgatgagagatcctaatccgcttgtggctggaagagggatcagcatgatgaaagaagctggcattgaggtaagggaaggcatcctggcagaccaggcggagaggctgaatgaaaaatttctgcactttatgaggacaggccttccgtacgtcacgctaaaagcggctgccagccttgacggcaagatagctaccagcacgggtgacagcaaatggatcacgtcagaggctgcaagacaggatgctcagcaatacaggaaaacacaccaaagcattttagtcggagttggcacagtgaaagccgacaatccgagcttaacctgcagactgccgaatgtaacaaaacagccggttcgggtcatacttgataccgtactctcgattcctgaggacgctaaagtgatttgcgatcaaatagcgccgacatggatttttacgacggcacgcgcagacgaggaaaagaaaaaacggctttcagctttcggagtgaacatatttacacttgaaaccgagcgcattcaaattcctgatgttttgaagatcctagcggaagaaggcatcatgtcggtgtatgtggaaggcggttcagctgttcacggaagctttgtcaaagaaggctgttttcaagaaatcatcttctattttgcccctaaactaatcggaggaacgcatgctcccagcttaatctccggtgaaggttttcaatcaatgaaagatgtccccttattacaattcactgatataacccaaatcggccgtgatatcaaactgacggcaaaaccgacaaaggaataggatggtgaccatgtttacaggaattatcgaagaaacaggcacaatcgaatccatgaaaaaagcagggcatgcaatggccttaactattaaatgctcaaagattttagaggatgttcatcttggcgacagcattgcagtgaacggcatttgtctgactgtcactgattttacaaaaaatcaattcacagtggatgttatgcctgaaacagtcaaagctacgtcactgaatgatttaacaaaaggaagcaaagtaaatctggaaagagcgatggcggcaaacggccgtttcggaggccatttcgtctcaggccatgtcgacggaactgcggaaatcacacgaattgaagagaaaagcaacgcagtttactatgatttaaaaatggacccgtcattaacaaaaacattggttttaaagggatcaattactgtggatggcgtgagcttaaccatattcggcctgacagaagacacagtgacgatctccttaataccgcatacgatcagcgaaacgatcttttcagaaaaaacgatcggctctaaagtgaatatcgaatgcgatatgatcggaaaatatatgtatcgatttttgcataaagccaatgaaaataagacccaacaaaccattacaaaagccttcttaagcgaaaacggcttttagagaggaagatttgcatgtttcatccgatagaagaagcactggacgctttaaaaaaaggcgaagtcatcatcgttgtagatgatgaagacagagaaaatgaaggagactttgtggctcttgccgagcatgcaacgccggaagtcattaactttatggcgacacatgggagaggactgatctgcacgccgctcagtgaggaaatcgcagacaggcttgatcttcaccctatggttgagcataatacagactctcaccacactgcatttaccgtaagcatagaccatcgtgaaacgaagacaggtatcagcgctcaagaaagatcttttaccgttcaagcattgctggacagcaaatccgtgccatctgattttcagcgtccggggcacatttttccactgattgcgaaaaaaggaggtgtcctgaaaagagcgggccatacagaagctgctgttgatcttgctgaagcttgcggatctccaggagccggcgtcatttgtgaaattatgaatgaagacggaacgatggcgagagtgcctgagctcattgaaattgcgaaaaagcatcaattaaaaatgatcaccattaaggatttgattcaataccgttacaatctgacaacacttgtcgagcgtgaagttgacattacgctgcctactgattttgggacatttaaggtttatggatacacaaatgaggtagatggaaaagagcatgtcgcatttgtgatgggagatgtgccgttcggagaagaaccggtattggtccgggtgcattcagaatgtctcacaggtgacgtgtttggctctcatcgctgtgattgcggaccgcagctgcacgccgcgctgaaccaaattgccgcagaaggccgtggagtgctcctgtacttgcgccaagaaggacgaggcatcggtttaatcaataaattaaaagcttataagcttcaggaacaaggctatgacaccgtagaagccaatgaggcgcttggattcttgccggatcttcgcaactatggcatcggagcacaaattttacgcgacctcggtgtccggaatatgaagcttttgacgaataatccgcgaaaaatcgcaggccttgaaggctacggactcagtatttcagaaagagtgccgcttcaaatggaggcgaaagaacacaataaaaaatatttgcaaaccaaaatgaacaagctaggtcatttacttcatttctaatcacaaatatcacaaaaaaggatgggaatcatatgaatatcatacaaggaaatttagttggtacaggtcttaaaatcggaatcgtagtaggaagatttaatgattttattacgagcaagctgctgagcggagcagaagatgcgctgctcagacatggcgtagacacaaatgacattgatgtggcttgggttccaggcgcatttgaaataccgtttgctgcgaaaaaaatggcggaaacaaaaaaatatgatgctattatcacattgggcactgtcatcagaggcgcaacgacacattacgattatgtctgcaatgaagctgcaaaaggcatcgcgcaagcagcaaacactactggtgtacctgtcatctttggaattgtaacaactgaaaacatcgaacaggctatcgagcgtgccggcacaaaagcgggcaacaaaggtgtagattgtgctgtttctgccattgaaatggcaaatttaaaccgctcatttgaataatttgctgaaaacagtttaaaaatatggcgaaaatgatataatgtgagaaaacggatcacctattcgtatccgttaatagcagactggacattttggatatagaggggtttttatgttaattcgttataaaaaatcgtttgaaaagattgcgatggggcttctttcgtttatgccgaatgaaaaagaccttaagcagcttcagcagacaattaaggactacgaaacggatacagaccgccagctctttctttggaaagaggacgaggatatcgtcggagcaatcggagtcgaaaaaaaggattctgaggttgagatccggcatatcagtgtgaatccttctcatcgccatcaaggaatcggaaaacagatgatggatgctttaaagcatttattcaaaacgcaagtactggttccaaatgaattaacgcagagctttttcgaacgttgtcaaggtcagcaggatcaagacatttcatacaataattaagcagaggctgtgatcagtctctgcttttttttctgcgttctatttctttttcacgttcacggatgacgtcagtccgatcccgcaaacggtgtttgtcgataagaaatatgttgctgagtgcactgggctgcccccatgtatacttttttttcctgcattcgatcctgcatgcttcctccagtttctcatctttgattggcagtataatgcttttataggcagagacggtttcgatttgttcgtaaaccgattgcataagttcgagcaaacggccatgatcaagccctaagtcttcgactgcccggtgttctgcttgaagaatccggatgctgttcgccatcagtctttttgccccggctgtattctgccttctgtgatgatataaagccactgcaagctgaataaagcccacccaatagcgttttcgtttctttggcggatcttccttccaatattcttctaatatttcatggcattcaaaataatcccgtgtcgcatgaaactcaacgagataatctatataagctttcggatacaaggtgcttcctcctttaatgtgcttttagtgtaccatagaagggataaaaggaaaaaagaacgcccggcctctagtgcttcttagaaaggaaaaccagaagcaaaggctttttctcccgcttctgcgctccgagcaaacacctcttgtgttttgaatattctgtacaaactccttcaaaacaggatatgaaatagtattggacgagagctttttggtggcttatactatagggtagccagtttttccggcaatgagagtgatacttgaaaatggtgagatgatggaagaatatcaagtgaaaattgatacgtttgagca

SEQ ID NO: 56 - DNA, 591 bp, Escherichia coli
atgcagcttaaacgtgtggcagaagccaaactgccaaccccatggggcgatttcctgatggtgggatttgaagaactggcaaccggacacgatcatgtcgcgctagtctatggcgatatttccgggcataccccggtacttgcgcgcgtccattccgaatgtctgaccggtgacgccctgttcagcttgcgctgcgattgtggcttccagctcgaagcggcattgacgcaaattgccgaggaaggccgtggtattttgctgtatcaccgtcaggaaggtcgtaacattggtctgctgaataaaatccgcgcttacgcactgcaggatcaaggttacgataccgtagaggctaaccaccagttaggcttcgccgctgatgagcgcgacttcactctttgcgctgatatgttcaaactccttggcgtcaatgaagtccgcttgttaaccaataacccgaaaaaagtcgaaattctgaccgaagcagggattaatattgttgaacgcgtaccattgattgtaggtcgtaaccccaataacgaacattatctcgataccaaagccgagaaaatgggccatttgctgaacaaataa

SEQ ID NO: 57 - DNA, 642 bp, Escherichia coli
atgtttacggggattgtacagggcaccgcaaaactggtgtcgattgacgagaaaccaaattttcgtacgcatgtggtggagttacccgaccacatgctggacggcctggaaaccggtgcttccgtggcgcataacggttgctgcctgaccgtgacggaaattaacggcaaccatgtcagttttgacctgatgaaagaaacgttacgcattaccaatcttggcgatttaaaagtgggggattgggtaaacgttgagcgtgcggcgaaattcagtgatgaaattggcggacacttaatgtcaggtcatattatgaccactgctgaagtggcgaaaatattaacctcagaaaataatcgccagatctggtttaaagtccaggatagtcagttgatgaaatatattctgtacaaaggatttattggcatcgacggtattagcctgaccgtcggcgaagtcacgccaacgcgtttttgcgtccatttaattccggaaacactggaacgcacgactcttgggaagaaaaaacttggcgcacgcgtcaacattgaaatcgatccacaaactcaggcagtggtagatacggtagaacgtgtgctggcggcacgagaaaatgccatgaatcaaccaggcacagaagcctga

SEQ ID NO: 58 - DNA, 3490 bp, Photobacterium leiognathi; identical to SEQ ID NO: 48

SEQ ID NO: 59 - DNA, 1438 bp, Arabidopsis thaliana
  misc_feature (39)..(39): n is a, c, g, or t
cgtcaatagaataaaaaaaactgtatgaatagaataatncgtttaaattggcacgtgaaatgcgaaatgggctgagtcattcattatggaaagcccatgtgattggtagggaacgacgtcgggtgaagggaggggaagagcttataaatgaaagcagcaagcagagcagttcacgctctttgtacactccgccctctctccccatctctttcgacagatctcttctctctctcgtgtttcacgaaacaatggcgactcaattcagcgcttctgtctcattgcaaacttcttgtctggtaatcaaatcttcttctttcattttaacaattgccttagagatcgctttgatcttacgattcatgatcatttcccgttgtttccatgcctgctcttgttatcgtatataaactcctgacgcatgtttattatgtgtaacatagaagatcttcgctttagtaatgatcagttttatgtttttgttccaatgattttatttctagtttcaaattctgtgatcggcaacacatggaattaaaatccgtggttttgtgttctggttgtaatctagatctacagtcaattgaatttatgattgtggaagtgaatgcttacttttggctgtgaataattgtgcatggacattcattggctaagaatttggcttgagttatgaaaattcagaacaaagttcttatgaatgcaatgctttttttttttgtgtgtgtttgatgaaacaaataggcaacaacaaggattagtttccaaaagccagctttgatttccaaccatggaaagactaatctatccttcaacctccgccgttcaatcccatctcgccgcctctctgtttcttgcgcggtatgagcattttttttcaagtttgttaatctatatatatagggctcaaagttttaaaagacattatatttggtttataggcaaaacaagagacgatagagaaagtgtctgctatagttaagaagcaactatcacttacaccggataaaaaagtcgttgcagaaaccaaatttgctgaccttggagcagattctctcgacacgtatccattcactcaagtggcaattaacacaaattcttaattttcttgatactgatcaactgagtgtttttcttcaggttgagatagtaatgggtttagaggaagagtttaacatccaaatggccgaagagaaagcacagaagattgccacagttgagcaagctgctgaactcattgaagagctcatcaacgagaagaagtaattttagctttataaaatgcccttaaataaactaaaaaagaaaacgacaaaaaaaaacagggcgttgagtttgttttcattatgtttgatttctctgtcattttctttaatgtgtctagcgagtctgcctttgtcccaatggtttagtatctgcatgtatccacggatctctattt

SEQ ID NO: 60 - DNA, 255 bp, Artificial Sequence
  Synthetic nucleotide sequence Photobacterium sp. SKA34 ACP
atgacagaagcgaatcgtaaccaagtttttgaccaagtaaaaaatgttcttgttgaactgtttgagcttgacgcagatgacattcaaccgcaaaccaatctatacactgaacttgatttagacagtattgatgccgttgatttagtcgttcacttacaaaacatgaccggtcagaaaatcaaaccagaagagttcaaagctgtccgtaccgttgatgacgtggtcgatgcagtgatcgagcttgttaaggaataa

SEQ ID NO: 61 - DNA, 26 bp, Artificial Sequence
  Synthetic nucleotide sequence T7 promoter
cgaaattaatacgactcactataggg

SEQ ID NO: 62 - DNA, 2652 bp, Artificial Sequence
  Synthetic nucleotide sequence T7 polymerase
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 63 - DNA, 3860 bp, Artificial Sequence
  Synthetic nucleotide sequence pSAT4-MCS
gcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggtccggactcagatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctc

SEQ ID NO: 64 - DNA, 95 bp, Artificial Sequence; identical to SEQ ID NO: 32
  Synthetic nucleotide sequence truncated Prrn promoter

SEQ ID NO: 65 - DNA, 792 bp, Artificial Sequence
  Synthetic nucleotide sequence aadA gene
atgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 66 - DNA, 21 bp, Artificial Sequence
  Synthetic nucleotide sequence rbcL leader sequence
agttgtagggagggatttatg

SEQ ID NO: 67 - DNA, 3870 bp, Artificial Sequence
  Synthetic nucleotide sequence Vector pCAS3- aadA
gcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtcgccgtcgttcaatgagaatggataagaggctcgtgggattgacgtgagggggcagggatggctatatttctgggagcgaactccgggcgaatatccatggagttgtagggagggatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaagatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctc

SEQ ID NO: 68 - DNA, 6547 bp, Photobacterium leiognathi
ccaaaggagattacatgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtagaaagagatatatcatggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaaatagtacttacctaagtacagccaaaaggaagaaataatgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaagctgttctttttaaactattcaatatcaaggcataaggaataaaatatgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataatttaattacggatagatattttcgatatatctaagtcttactaccatttatataaactatttatacagataacgtttcatttgattaagtcagtaaataattgccattaattaatggcagtgcagatccttacactgccatttataaattaaataagggttaacatgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataaaagcagggcttaatcatgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaa

SEQ ID NO: 69 - DNA, 10423 bp, Artificial Sequence
  Synthetic nucleotide sequence Vector pCAS3- aadA-LUXoperon
gcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtcgccgtcgttcaatgagaatggataagaggctcgtgggattgacgtgagggggcagggatggctatatttctgggagcgaactccgggcgaatatccatggagttgtagggagggatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaagatctcgagctcaagcttcgaattcccaaaggagattacatgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtagaaagagatatatcatggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaaatagtacttacctaagtacagccaaaaggaagaaataatgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaagctgttctttttaaactattcaatatcaaggcataaggaataaaatatgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataatttaattacggatagatattttcgatatatctaagtcttactaccatttatataaactatttatacagataacgtttcatttgattaagtcagtaaataattgccattaattaatggcagtgcagatccttacactgccatttataaattaaataagggttaacatgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataaaagcagggcttaatcatgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaagaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctc

SEQ ID NO: 70 - DNA, 1977 bp, Artificial Sequence
  Synthetic nucleotide sequence rps12 homologues recombination sequence
gaagtgcttcgaatcattgctatttgactcggacctgttctaaaaaagtcgaggtatttcgaattgtttgttgacacggacaaagtcagggaaaacctctgaaattatttcaatattgaaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgatcctttactccgacagcatctagggttcctcgaacaatgtgatatctcacaccgggtaaatccttaacccttccccctcttactaagactacagaatgttcttgtaaattatggccaataccgggtatataagcagtgatttcaaatccagaggttaatcgtactctggcaactttacgtaaggcagagtttggtttttttggggtgatagtggaaaagttgacagataagtcacccttactgccactctacagaaccgtacatgagattttcacctcatacggctcctcgttcaattctttcgaattcattggatcctttccgcgttcgagaatcccccccttcttccactccgccccgaagagtaactaggaccaatttagtcacgttttcatgttccaattgaacactgtccatttttgattattctcaaaggataagattattctctttaccaaacatatgcggatccaatcacgatcttatatataagaagaacaaaagatctttcttgatcaatccctttgcccctcattcttcaagaataaggaagatccttttcaagtttgaatttgttcatttggaatctgggttcttctacttcatatttatttaatatgaatattttccctctcttttttttatatcattccttaagtcccataggtttgatcctgtagaattttacccattttctcattgaacgaaaggtacgaaataaatcagattgataaaagtaccatgtgaaatcttcggtttttccccttcctcgatccctatcccataggttaggtacagtgtttgaatcaatagagaaccttttcttctgtatgaatcgatattattccattcaaaatccttcccgatacctcccaaggaaaatctcgaatttggatcccaaattgacgggttagtgtgagcttatccatgcggttatgcactctttgaataggaatccgttttctgaaagatcctggctttcgtactttggtgggtctccgagatcctttcgatgacctatgttgaagggatatctatctaatccgatcgattgcgtaaagccagcggtagcaacggaaccggggaaagtatacagaaaagacagttcttttctattatattagtattttctattatattagattagtattagttagtgatcccgacttagtgagtctgatgaattgttggcaccagtcctacattttgtctctgtggaccgaggagaaaaggggctcggcgggaagaggagtgtaccatgagagaagcaaggaggtcaacctctttcaaatatacaacatggattctggcaatgtagttggactctcatgtcgatccgaatgaatcatcctttccacggaggtaaatctttgcctgctaggcaagaggatagcaagttccaaattctgtctcggtaggacatgtatttctattactatgaaattcataaatgaagtagttaatggtagggttaccattatcctttttgtagtgacgaatcttgtatgtgttcctaagaaaaggaatttgtccatttttcggggtctcaaaggggcgtggaaacgcataagaactcttgaatggaaaagagatgtaactccagttccttcggaatcggtagtcaatcctatttccgataggggcagttgataa

SEQ ID NO: 71 - DNA, 1986 bp, Artificial Sequence
  Synthetic nucleotide sequence TrnV homologues recombination sequence
caattgaatccgattttgaccattattttcatatccgtaatagtgcgaaaagaaggcccggctccaagttgttcaagaatagtggcgttgagtttctcgaccctttgacttaggattagtcagttctatttctcgatggggcggggaagggatataactcagcggtagagtgtcaccttgacgtggtggaagtcatcagttcgagcctgattatccctaagcccaatgtgagtttttctagttggatttgctcccccgccgtcgttcaatgagaatggataagaggctcgtgggattgacgtgagggggcagggatggctatatttctgggagcgaactccgggcgaatatgaagcgcatggatacaagttatgccttggaatgaaagacaattccgaatccgctttgtctacgaacaaggaagctataagtaatgcaactatgaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcac

SEQ ID NO: 72 - DNA, 14422 bp, Artificial Sequence
  Synthetic nucleotide sequence Vector pCA3- LUX-rps12/TrnV
gcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtgaagtgcttcgaatcattgctatttgactcggacctgttctaaaaaagtcgaggtatttcgaattgtttgttgacacggacaaagtcagggaaaacctctgaaattatttcaatattgaaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgatcctttactccgacagcatctagggttcctcgaacaatgtgatatctcacaccgggtaaatccttaacccttccccctcttactaagactacagaatgttcttgtaaattatggccaataccgggtatataagcagtgatttcaaatccagaggttaatcgtactctggcaactttacgtaaggcagagtttggtttttttggggtgatagtggaaaagttgacagataagtcacccttactgccactctacagaaccgtacatgagattttcacctcatacggctcctcgttcaattctttcgaattcattggatcctttccgcgttcgagaatcccccccttcttccactccgccccgaagagtaactaggaccaatttagtcacgttttcatgttccaattgaacactgtccatttttgattattctcaaaggataagattattctctttaccaaacatatgcggatccaatcacgatcttatatataagaagaacaaaagatctttcttgatcaatccctttgcccctcattcttcaagaataaggaagatccttttcaagtttgaatttgttcatttggaatctgggttcttctacttcatatttatttaatatgaatattttccctctcttttttttatatcattccttaagtcccataggtttgatcctgtagaatttgacccattttctcattgaacgaaaggtacgaaataaatcagattgataaaagtaccatgtgaaatcttcggtttttccccttcctcgatccctatcccataggttaggtacagtgtttgaatcaatagagaaccttttcttctgtatgaatcgatattattccattccaaatccttcccgatacctcccaaggaaaatctcgaatttggatcccaaattgacgggttagtgtgagcttatccatgcggttatgcactctttgaataggaatccgttttctgaaagatcctggctttcgtactttggtgggtctccgagatcctttcgatgacctatgttgaagggatatctatctaatccgatcgattgcgtaaagcccgcggtagcaacggaaccggggaaagtatacagaaaagacagttcttttctattatattagtattttctattatattagatatattagactattatattagattagtattagttagtgatcccgacttagtgagtctgatgaattgttggcaccagtcctacattttgtctctgtggaccgaggagaaaaggggctcggcgggaagaggagtgtaccatgagagaagcaaggaggtcaacctctttcaaatatacaacatggattctggcaatgtagttggactctcatgtcgatccgaatgaatcatcctttccacggaggtaaatctttgcctgctaggcaagaggatagcaagttccaaattctgtctcggtaggacatgtatttctattactatgaaattcataaatgaagtagttaatggtagggttaccattatcctttttgtagtgacgaatcttgtatgtgttcctaagaaaaggaatttgtccatttttcggggtctcaaaggggcgtggaaacgcataagaactcttgaatggaaaagagatgtaactccagttccttcggaatcggtagtcaatcctatttccgataggggcagttgataaaccggtcgccgtcgttcaatgagaatggataagaggctcgtgggattgacgtgagggggcagggatggctatatttctgggagcgaactccgggcgaatatccatggagttgtagggagggatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaagatctcgagctcaagcttcgaattcccaaaggagattacatgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtagaaagagatatatcatggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaaatagtacttacctaagtacagccaaaaggaagaaataatgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaagctgttctttttaaactattcaatatcaaggcataaggaataaaatatgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataatttaattacggatagatattttcgatatatctaagtcttactaccatttatataaactatttatacagataacgtttcatttgattaagtcagtaaataattgccattaattaatggcagtgcagatccttacactgccatttataaattaaataagggttaacatgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataaaagcagggcttaatcatgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaagaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgccaattgaatccgattttgaccattattttcatatccgtaatagtgcgaaaagaaggcccggctccaagttgttcaagaatagtggcgttgagtttctcgaccctttgacttaggattagtcagttctatttctcgatggggcggggaagggatataactcagcggtagagtgtcaccttgacgtggtggaagtcatcagttcgagcctgattatccctaagcccaatgtgagtttttctagttggatttgctcccccgccgtcgttcaatgagaatggataagaggctcgtgggattgacgtgagggggcagggatggctatatttctgggagcgaactccgggcgaatatgaagcgcatggatacaagttatgccttggaatgaaagacaattccgaatccgctttgtctacgaacaaggaagctataagtaatgcaactatgaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacgcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctc

SEQ ID NO: 73 - DNA, 1627 bp, Artificial Sequence
  Synthetic nucleotide sequence TrnI homologues recombination sequence
cttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctgcgccagggaaaagaatagaagaagcatct

SEQ ID NO: 74 - DNA, 1625 bp, Artificial Sequence
  Synthetic nucleotide sequence TrnA homologues recombination sequence
actacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagg

SEQ ID NO: 75 - DNA, 13689 bp, Artificial Sequence
  Synthetic nucleotide sequence Vector pCA3- LUX-TrnI/TrnA
gcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccattaccctgttatccctaaccggtcttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccccaaaaaaaagaagggagctacgtctgagttaaacttggagatggaagtcttctttcctttctcgacggtgaagtaagaccaagctcatgagcttattatcctaggtcggaacaagttgataggacccccttttttacgtccccatgttccccccgtgtggcgacatgggggcgaaaaaaggaaagagagggatggggtttctctcgcttttggcatagcgggcccccagtgggaggctcgcacgacgggctattagctcagtggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaactcctcctcaggaggatagatggggcgattcgggtgagatccaatgtagatccaactttcgattcactcgtgggatccgggcggtccgggggggaccaccacggctcctctcttctcgagaatccatacatcccttatcagtgtatggacagctatctctcgagcacaggtttagcaatgggaaaataaaatggagcacctaacaacgcatcttcacagaccaagaactacgagatcgcccctttcattctggggtgacggagggatcgtaccattcgagccgtttttttcttgactcgaaatgggagcaggtttgaaaaaggatcttagagtgtctagggttgggccaggagggtctcttaacgccttcttttttcttctcatcggagttatttcacaaagacttgccagggtaaggaagaaggggggaacaagcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccagctgcgccagggaaaagaatagaagaagcatctaccggtcgccgtcgttcaatgagaatggataagaggctcgtgggattgacgtgagggggcagggatggctatatttctgggagcgaactccgggcgaatatccatggagttgtagggagggatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaagatctcgagctcaagcttcgaattcccaaaggagattacatgattaagaagatcccaatgattattgggggtgtagttcaaaacacgtctggatatggcatgcgtgaactaacgctcaacaataataaagtgaatatccctatcatcacccaaagtgatgttgaagctattcaatcactaaatatagaaaacaaattgactataaatcagatagttaatttcttatatacagtgggacaaaaatggaagagcgaaacttacagccgacgactcacttatattcgagatcttattaagttcctcggttactcacaagagatggcaaaacttgaagctaactggatctcaatgattctgtgtagcaaaagtgcgttgtacgatattgttgagaatgatcttagctcacggcatattattgatgagtggatcccccaaggtgaatgttatgtcaaagcgctcccaaaaggaaaatctgtacacctattagctggtaacgtaccactatctggtgtgacttctattcttcgtgcgattttgaccaaaaacgagtgcatcataaaaacgtcatcagctgatccttttacagctactgcgctagttaatagttttatcgatgtagatgcagaacacccgatcacacgttcaatctcagttatgtattggtcacatagcgaggatcttgctattccaaaacaaataatgagctgtgctgatgtggttattgcatggggtggtgatgatgcaattaaatgggctacagaacatgcaccatcacacgcagatattctaaaatttggtcccaaaaagagtatatccattgttgacaacccaacagatattaaggctgctgctatcggtgtagcacatgatatctgtttttacgatcagcaagcatgtttctccacccaagatatttattatattggcgatagcatagacatattttttgatgaattagctcagcaattaaataaatataaagacatattgcctaaaggtgagcggaattttgatgaaaaagcagctttttctttaacggaaagagaatgtttgtttgccaaatataaagttcaaaaaggtgaaagccaatcttggttattaacgcaatcacctgcgggatcatttggtaatcagccgttatcacgctcggcttatattcatcaagtaaatgacatttcagaagtcattccattcgtgcataaggcggtaacgcaaaccgtcgcaatagcgccgtgggagtcgtctttcaaatatagagatatattagcagaacatggtgcagaacgaattatagaagccggaatgaataatatatttcgagtaggtggcgcccatgatgggatgcgtccccttcaacggcttgttaactatatatcacatgaaaggccgtcaacatataccactaaagatgtctcggtgaaaatcgaacagactcgttatcttgaggaagataagttcctcgtatttgtaccgtagaaagagatatatcatggaaaatacacaacattcattacctattgatcacgtaattgatattggtgataaccgttatattcgagtatgggaaaccaagccgaaaaataaagaaaccaagcgtaataataccatcgttatcgcctcaggctttgctcgacgcatggatcattttgctggtcttgccgaatatttagcaaataatggttttcgtgttattcgttatgattcgttaaatcatgtcggtcttagtagcggagagatcaaacagttctcgatgtcagtaggtaaacacagtttgctaactgttattgattggctaaaagaacgaaatattaacaatataggtcttattgcttcgagtctttctgctcgaattgcttatgaagtggcagcagaaattgatttgtcatttttaattaccgccgtcggtgttgtcaatttaagaagtacgctagaaaaagcactgaaatatgattatctacaaatggaagtaaatactattcctgaagatttaatttttgaaggacacaatctaggttcaaaagtctttgtgacagattgttttgaaaataattgggactcattagattcgacaataaataaaatttgtgaactagatattccatttattgctttcacttcagatggcgatgattgggtttgccaacatgaagtaaaacatttagtcagtaacgttaaatctgacaaaaagaaaatttactcactcgttggctcatctcatgatttgggcgaaaacctagtggtgcttcgtaacttctatcaatcaatgacgaaagctgctgtgagcttagatcgtcaattagtagagcttgttgatgaaattattgaaccaaattttgaagacctaacagttattacggtaaatgaacggcgcctcaaaaataaaatcgaaaatgaaattattaatagattagctgatcgcgtattggctagtgtctaaatagtacttacctaagtacagccaaaaggaagaaataatgaaaattagtaatatctgtttctcataccaaccaccaggtgaatcacatcaagaggtaatggagcgctttattcgtttaggcgttgcatcagaagagctcaactttgatggtttctatacacttgaacaccatttcactgagtttggtattacaggtaacctttatattgcctgtgccaatattcttggtcgaaccaaaaggatccaagtcggtaccatggggatagtgttaccgacagagcacccagcacgacatgtagaaagtcttctcgttttagatcaactgtctaaagggcgctttaactacggtactgttcgcggactctaccataaagattttcgtgtttttggtacatcacaggaagattctcgtaagaccgcagaaaatttctactctatgatcttggatgcatcaaaaacaggtgtgctacatactgacggtgaagtagtagagttcccagatgtcaatgtttatccagaagcttacagcaaaaaacaacccacctgcatgacagccgaatcatccgagaccatcacttatttagctgaacgtggtttaccaatggtgttaagttggattattccggtcagtgagaaagtctcacaaatggaattgtacaatgaagttgcggcagagcatggtcatgacattaacaacattgaacatatcctaactttcatttgctctgtaaatgaagacggtgaaaaagcagacagcgtatgccgtaatttcctagaaaattggtacgactcttacaaaaatgcaaccaacatcttcaacgacagtaaccaaactcgtggctacgattacctcaaagctcagtggcgtgagtgggtaatgaaggggttggctgatcctcgtcgccgacttgattacagtaacgaattaaaccctgtcggcacgccagaacgatgcattgagatcattcaaagtaatattgatgccactggaattaagcatattactgttggatttgaagcgaatggttctgaacaagaaattcgtgaatccatggagctatttatggaaaaagtagcgccacacttaaaagatcctcagtaagctgttctttttaaactattcaatatcaaggcataaggaataaaatatgaatttcgggttatttttcctaaatttccagcctgaaggtatgacttcagaaatggttttagacaacatggtagatactgtcgcattagtggataaagatgattaccactttaaaagagtgctcgtcagcgagcatcatttttctaaaaacggcattatcggagaacctttgacagcgattagcttcttacttggtttgactaaacgtatagaaattggttctttaaatcaagtgattaccacccatcatcctgtacgtatcggagaacaaacgggcttacttgatcaaatgtcttacggtcgtttcgttttaggcttaagtgactgtgtcaatgacttcgaaatggatttctttaagagaaaacgtagctctcaacagcaacaattcgaagcatgttacgaaattttaaatgaagcgctgacgacaaactattgtcaggcagatgatgacttctttaacttcccacgtatttctgttaacccgcattgtattagcgaagtaaaacaatatattttagcttcaagcatgggcgtggttgaatgggcagcaagaaaaggattgccactcacttaccgctggagtgacagcctagcagaaaaagaaaaatactatcagcgttatctcgctgttgctaaagagaataatattgatgtatcaaatattgaccaccaattcccactgctcgttaatatcaatgaaaatcgtcgtattgctcgagatgaagtaagggagtatatacaaagttatgtgagtgaagcctaccctactgaccccaacattgagctaagagtagaagagcttattgagcagcatgctgtcggcaaagtggatgagtactacgactcaacaatgcacgcagtaaaagttacaggttcaaaaaatttattactctcttttgaatcaatgaaaaataaagacgatgttaccaagcttataaatatgtttaatcaaaaaatcaaagataaccttattaaataatttaattacggatagatattttcgatatatctaagtcttactaccatttatataaactatttatacagataacgtttcatttgattaagtcagtaaataattgccattaattaatggcagtgcagatccttacactgccatttataaattaaataagggttaacatgtcaacattattaaatatagatgcaactgaaattaaggtgagtacagaaatagatgatattatttttacatcatcaccgctaacgttactatttgaagatcaagaaaaaatacagaaagaacttattttggagtctttccattatcattacaatcataataaagattataagtactattgtaatatacaaggcgtagatgagaatatacagtccattgacgatattcctgtttttcctacttcaatgttcaagtactcaagattacatactgctgatgaatcaaatattgaaaattggtttactagtagtggtacaaagggagtcaaaagtcatatagctcgagatcggcagagtattgaacgcttgctaggttctgttaattacggcatgaaatacttgggtgaatttcacgagcatcaattagaactagtgaatatggggccagatcgtttcagtgcgtcaaatgtttggtttaaatatgtaatgagcttagttcaattactttacccaacaacatttaccgttgaaaacgatgaaatcgattttgaacaaaccatcttagcgttaaaagcaattcagcgtaaaggaaaaggaatttgtttaattggccctccgtattttatttatttgttatgccactacatgaaagagcataatatcgaatttaatgctggtgcacatatgtttatcattacaggtgggggatggaaaaccaaacaaaaagaagcgctaaaccgacaagatttcaatcaactattgatggagacttttagccttttccatgaaagtcaaattcgagatatctttaaccaagtagagctaaacacttgtttctttgaagacagcctacagcgtaaacatgtaccaccgtgggtatatgctcgtgcgcttgatcctgtcactttaacgcccgtagaagatggccaagagggcttgatgagttatatggatgcctcatctaccagctacccgacatttattgttaccgacgatattggtattgttcgccatctaaaagaaccagatccattccaaggaacaacggttgaaattgttcgtcgtttaaatacgcgagaacaaaaaggatgttcactctcaatggccacgagcctgaaataaaagcagggcttaatcatgatttttaattgcaaggttaaaaaagtcgaagcatctgacagccatatttacaaagtgtttattaagcctgacaaatgctttgattttaaagcgggtcaatatgtaattgtgtatctcaatggaaaaaatttgccgttttctattgctaactgcccaacttgtaatgagctccttgaattacatgtaggaggttcggtaaaagaatccgccattgaagctatttcgcactttattaatgcatttatttatcaaaaagaatttacaatcgatgcaccacacggtgatgcatggctgagagatgaaagccaatcacctttactacttatagcaggagggacaggtttatcatatatcaatagcattttaagttgttgtattagtaaacagttatctcagcctatctatctttattggggagtaaataactgtaatttactctatgctgatcaacaactaaaaacactcgccgcacaatacagaaatataaattatattcctgtggtagagaatttaaatactgactggcagggaaaaattggtaatgttattgacgcggttattgaagatttttcagatttatctgactttgatatctatgtctgcgggccatttggtatgagccggactgcgaaagatattctgatctcacagaaaaaggcgaatataggaaaaatgtattctgatgcatttagctatacgtaagaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcactacttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacgtgccactgaaagactctactgagacaaagatgggctgtcaagaacgtagaggaggtaggatgggcagttggtcagatctagtatggatcgtacatggacggtagttggagtcggcggctctcccagggttccctcatctgagatctctggggaagaggatcaagttggcccttgcgaacagcttgatgcactatctcccttcaaccctttgagcgaaatgcggcaaaagaaaaggaaggaaaatccatggaccgaccccatcatctccaccccgtaggaactacgagatcaccccaaggacgccttcggcatccaggggtcacggaccgaccatagaaccctgttcaataagtggaacgcattagctgtccgctctcaggttgggcagtcagggtcggagaagggcaatgactcattcttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctttatgatagcacccaatttttccgattcggcggttcgatctatgatttatcattcatggacgttgataagatccatccatttagcagcaccttaggatggcatagccttaaaagtgaagggcgaggttcaaacgaggaaaggcttacggtggatacctaggcacccagagacgaggaagggcgtagtaatcgacgaaatgcttcggggagttgaaaataagcatagatccggagattcccgaatagggcaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagcccgaatgctgcaccctagatggcgaaagtccagtagccgaaagcatcactagcttatgctctgacccgagtagcatggggcacgtggaatcccgtgtgaatcagcaaggaccaccttgcaaggctaaatactcctgggtgaccgatagcgaagtagtaccgtgagggaagggtgaaaagaacccccatcggggagtgaaatagaacatgaaaccgtaagctcccaagcagtgggaggagccagggctctgaccgcgtgcctgttgaagaatgagccggcgactcataggcagtggcttggttaagggaacccaccggagccgtagcgaaagcgagtcttcatagggcggccgcattaccctgttatccctagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctc

SEQ ID NO: 76 - DNA, 30 bp, Artificial Sequence
  Synthetic nucleotide sequence primer
tcaccggtcgccgtcgttcaatgagaatgg

SEQ ID NO: 77 - DNA, 29 bp, Artificial Sequence
  Synthetic nucleotide sequence
gagcgaactccgggcgaatatccatggtt

SEQ ID NO: 78 - DNA, 47 bp, Artificial Sequence
  Synthetic nucleotide sequence
aaccatggagttgtagggagggatttatgggggaagcggtgatcgcc

SEQ ID NO: 79 - DNA, 34 bp, Artificial Sequence
  Synthetic nucleotide sequence
tggagatctttatttgccgactaccttggtgatc

SEQ ID NO: 80 - DNA, 32 bp, Artificial Sequence
  Synthetic nucleotide sequence
acagaattcccaaaggagattacatgattaag

SEQ ID NO: 81 - DNA, 32 bp, Artificial Sequence
  Synthetic nucleotide sequence
ttggaattcttacgtatagctaaatgcatcag

SEQ ID NO: 82 - DNA, 39 bp, Artificial Sequence
  Synthetic nucleotide sequence
agttagaaccggtgaagtgcttcgaatcattgctatttg

SEQ ID NO: 83 - DNA, 40 bp, Artificial Sequence
  Synthetic nucleotide sequence
cgatctaaccggtttatcaactgcccctatcggaaatagg

SEQ ID NO: 84 - DNA, 43 bp, Artificial Sequence
  Synthetic nucleotide sequence
ataatgcggccgccaattgaatccgattttgaccattattttc

SEQ ID NO: 85 - DNA, 39 bp, Artificial Sequence
  Synthetic nucleotide sequence
attatgcggccgcgtgaagcagtgtcaaaccaaaatacc

SEQ ID NO: 86 - DNA, 37 bp, Artificial Sequence
  Synthetic nucleotide sequence
agttagaaccggtcttcgggaacgcggacacaggtgg

SEQ ID NO: 87 - DNA, 39 bp, Artificial Sequence
  Synthetic nucleotide sequence
cgatctaaccggtagatgcttcttctattcttttccctg

SEQ ID NO: 88 - DNA, 40 bp, Artificial Sequence
  Synthetic nucleotide sequence
ctattatgcggccgcactacttcatgcatgctccacttgg

SEQ ID NO: 89 - DNA, 40 bp, Artificial Sequence
  Synthetic nucleotide sequence
gaatgatgcggccgccctatgaagactcgctttcgctacg

SEQ ID NO: 90 - DNA, 20 bp, Artificial Sequence
  Synthetic nucleotide sequence
ttgagtatccgtttccctcc

SEQ ID NO: 91 - DNA, 25 bp, Artificial Sequence
  Synthetic nucleotide sequence
ccagcaaatcaatatcactgtgtgg

SEQ ID NO: 92 - DNA, 22 bp, Artificial Sequence
  Synthetic nucleotide sequence
aagctcatgagcttggtcttac

SEQ ID NO: 93 - DNA, 27 bp, Artificial Sequence
  Synthetic nucleotide sequence
cagatttatctgactttgatatctatg

SEQ ID NO: 94 - DNA, 22 bp, Artificial Sequence
  Synthetic nucleotide sequence
atgaatttcgggttatttttcc

SEQ ID NO: 95 - DNA, 22 bp, Artificial Sequence
  Synthetic nucleotide sequence
ttatttaataaggttatctttg

SEQ ID NO: 96 - DNA, 22 bp, Artificial Sequence
  Synthetic nucleotide sequence
atgattaagaagatcccaatga

SEQ ID NO: 97 - DNA, 22 bp, Artificial Sequence
  Synthetic nucleotide sequence
ctacggtacaaatacgaggaac

SEQ ID NO: 98 - DNA, 29 bp, Artificial Sequence
  Synthetic nucleotide sequence
aattgaatccgattttgaccattattttc

SEQ ID NO: 99 - DNA, 22 bp, Artificial Sequence
  Synthetic nucleotide sequence
aagctcatgagcttggtcttac

SEQ ID NO: 100 - DNA, 25 bp, Artificial Sequence
  Synthetic nucleotide sequence
cgttcgcaagaatgaaactcaaagg

SEQ ID NO: 101 - DNA, 23 bp, Artificial Sequence
  Synthetic nucleotide sequence
cgctgattcttcaacatcagtcg